(19) United States
(12) Patent Application Publication
(10) Pub. No.: US 2022/0384027 A1
(43) Pub. Date: Dec. 1, 2022

(54) TRACKING AND REWARDING HEALTH AND FITNESS ACTIVITIES USING BLOCKCHAIN TECHNOLOGY

(71) Applicant: KC HOLDINGS I, Mayfield Village, OH (US)

(72) Inventors: Robert Louis Kaleal, III, Mayfield Village, OH (US); Dominic Guy Carbone, JR., Mayfield Village, OH (US)

(21) Appl. No.: 17/818,519

(22) Filed: Aug. 9, 2022

Related U.S. Application Data
(63) Continuation-in-part of application No. 17/806,117, filed on Jun. 9, 2022.
Application No. 17/806,117 is a continuation of application No. 17/389,047, filed on Jul. 29, 2021.
Application No. 17/389,047 is a continuation of application No. 15/978,625, filed on May 14, 2018, now Pat. No. 11,107,579.
Application No. 15/978,625 is a continuation of application No. 15/474,111, filed on Mar. 30, 2017, now Pat. No. 9,997,082.
Application No. 15/474,111 is a continuation of application No. 14/945,600, filed on Nov. 19, 2015, now Pat. No. 9,652,992.
Application No. 14/945,600 is a continuation-in-part of application No. 14/518,844, filed on Oct. 20, 2014, now Pat. No. 9,501,942.
Application No. 14/518,844 is a continuation-in-part of application No. 14/049,981, filed on Oct. 9, 2013, now Pat. No. 9,198,622.
(60) Provisional application No. 61/711,510, filed on Oct. 9, 2012.

(51) Int. Cl. G16H 40/63; G09B 5/02; A61B 5/00; A61B 5/20; G06Q 50/00; A61B 5/0205; G06T 19/00; G06F 1/16; G06Q 10/06; G06Q 10/10; G16H 20/70; A61B 5/11; A61B 5/16; G06T 13/40; G09B 5/06; G09B 19/00; G16H 20/40

(52) U.S. Cl. CPC G16H 40/63; G09B 5/02; A61B 5/486; A61B 5/45; A61B 5/40; A61B 5/42; A61B 5/41; A61B 5/20; A61B 5/43; G06Q 50/01; A61B 5/7264; A61B 5/744; A61B 5/7445; A61B 5/0205; G06T 19/00; G06F 1/1626; G06Q 10/0639; G06Q 10/10; G16H 20/70; A61B 5/02055; A61B 5/11; A61B 5/165; G06T 13/40; G09B 5/06; G09B 19/00; G09B 19/0092; G16H 20/40; A61B 5/44; A61B 5/08

(57) ABSTRACT

Systems and methods are disclosed that facilitate tracking and rewarding health and fitness activities of users using blockchain technology. In an aspect, a system comprises a non-transitory memory comprising instructions, and one or more hardware processors coupled to the non-transitory memory and configured to read the instructions to cause the system to perform operations. The operations comprise receiving health and fitness information for entities, employing a blockchain network to store the health and fitness information for the entities, and sending a non-fungible token (NFT) to an entity of the entities in response to a subset of the health and fitness information associated with the entity satisfying a criterion.

### CROSS-REFERENCE TO RELATED APPLICATIONS

This application is a continuation in-part of and claims priority to U.S. patent application Ser. No. 17/806,117 filed Jun. 9, 2022 and entitled “PERSONALIZED AVATAR RESPONSIVE TO USER PHYSICAL STATE AND CONTEXT,” which is a continuation of and claims priority to each of, U.S. patent application Ser. No. 17/389,047 filed on Jul. 29, 2021 and entitled “PERSONALIZED AVATAR RESPONSIVE TO USER PHYSICAL STATE AND CONTEXT,” which is a continuation of U.S. patent application Ser. No. 15/978,625 filed on May 14, 2018 (now U.S. Pat. No. 11,107,579) and entitled “PERSONALIZED AVATAR RESPONSIVE TO USER PHYSICAL STATE AND CONTEXT,” which is a continuation of U.S. patent application Ser. No. 15/474,111 filed on Mar. 30, 2017 (now U.S. Pat. No. 9,997,082) and entitled “PERSONALIZED AVATAR RESPONSIVE TO USER PHYSICAL STATE AND CONTEXT,” which is a continuation of U.S. patent application Ser. No. 14/945,600, filed on Nov. 19, 2015 (now U.S. Pat. No. 9,652,992) and entitled “PERSONALIZED AVATAR RESPONSIVE TO USER PHYSICAL STATE AND CONTEXT,” which is a continuation in-part of U.S. patent application Ser. No. 14/518,844, filed Oct. 20, 2014 (now U.S. Pat. No. 9,501,942) and entitled, “PERSONALIZED AVATAR RESPONSIVE TO USER PHYSICAL STATE AND CONTEXT,” which is a continuation in part of U.S. patent application Ser. No. 14/049,981, filed on Oct. 9, 2013 (now U.S. Pat. No. 9,198,622) and entitled “VIRTUAL AVATAR USING BIOMETRIC FEEDBACK,” which claims priority to U.S. Provisional Patent Application Ser. No. 61/711,510 filed on Oct. 9, 2012, and entitled “VIRTUAL AVATAR USING BIOMETRIC FEEDBACK.” The entireties of the aforementioned applications are incorporated by reference herein.

### TECHNICAL FIELD

This application generally relates to computer-implemented techniques for facilitating health and fitness and more particularly to tracking and rewarding health and fitness activities of users using blockchain technology.

### BACKGROUND

The use of personal biometric monitoring equipment has increased the ability of individuals to more easily and more accurately collect, track and analyze data relating to the body's response to various triggers. For example, wearable sensors can monitor heart rate during an exercise program and collect and record the heart rate data for further analysis. In addition, wearable movement sensors including fine tuned accelerometers and gyroscopes combined with pattern analysis have enabled the detection and analysis of user motion. The symbiosis of user motion and biometric analysis facilitates an acute understanding of an individual's physiological responses to various types of events and triggers. As a result, various tools can be established that help to control, improve or accommodate an individual's physical and physiological activity.

### BRIEF DESCRIPTION OF THE DRAWINGS

FIG. 1 illustrates an example system architecture for manifesting responses to physical and physiological activity information and image data about a user using a virtual avatar in accordance with various aspects and embodiments described herein.

FIG. 2 provides an example avatar guidance system in accordance with various aspects and embodiments described herein.

FIG. 3 illustrates a flow diagram of a process for guiding or assisting a user with adhering to a program, routine or activity using an avatar in accordance with various aspects and embodiments described herein.

FIG. 4 illustrates an example fitness module for employment with an avatar guidance system in accordance with various aspects and embodiments described herein.

FIG. 5 illustrates an example user interface that facilitates receiving user profile information in association with employment of an avatar guidance system for physical fitness purposes in accordance with various aspects and embodiments described herein.

FIG. 6 presents an example user interface that facilitates designing a custom fitness routine in association with employment of an avatar guidance system for physical fitness purposes in accordance with various aspects and embodiments described herein.

FIG. 7 presents an example user interface that presents a user with an avatar to facilitate guiding the user through a selected fitness routine in accordance with various aspects and embodiments described herein.

FIG. 8 illustrates another example fitness module for employment with an avatar guidance system in accordance with various aspects and embodiments described herein.

FIG. 9 illustrates an example program guide module for employment with an avatar guidance system in accordance with various aspects and embodiments described herein.

FIG. 10 presents a flow diagram of an example method for generating a personalized avatar that is responsive to a user's physical state and context, in accordance with various aspects and embodiments described herein.

FIG. 11 presents a flow diagram of another example method for generating a personalized avatar that is responsive to a user's physical state and context, in accordance with various aspects and embodiments described herein.

FIG. 12 presents a flow diagram of another example method for generating a personalized avatar that is responsive to a user's physical state and context, in accordance with various aspects and embodiments described herein.

FIG. 13 presents an example avatar visualization system that facilitates visualizing how the user will appear in response to adhering to a health and fitness program in accordance with various aspects and embodiments described herein.

FIG. 14 presents another example avatar visualization system that facilitates visualizing how the user will appear in response to adhering to a health and fitness program in accordance with various aspects and embodiments described herein.

FIG. 15 presents a flow diagram of an example method that facilitates visualizing how the user will appear in response to adhering to a health and fitness program in accordance with various aspects and embodiments described herein.

FIG. 16 presents a flow diagram of another example method that facilitates visualizing how the user will appear in response to adhering to a health and fitness program in accordance with various aspects and embodiments described herein.

FIG. 17 illustrates a high-level block diagram of an example, non-limiting system that can facilitate tracking and rewarding health and fitness activities of users using blockchain technology.

FIG. 18 illustrates example machine-executable components that can facilitate tracking and rewarding health and fitness activities of users using blockchain technology, in accordance with one or more embodiments described herein.

FIG. 19 illustrates an example computer-implemented method for tracking and rewarding health and fitness activities of users using blockchain technology, in accordance with one or more embodiments described herein.

FIG. 20 illustrates another example computer-implemented method for tracking and rewarding health and fitness activities of users using blockchain technology, in accordance with one or more embodiments described herein.

FIG. 21 illustrate an example system for facilitating a blockchain transaction.

FIG. 22 illustrates an example blockchain network.

FIG. 23 illustrates an example blockchain.

FIG. 24 is a diagram of an example transaction message.

FIG. 25 shows an example transaction broadcast the blockchain network.

FIG. 26 is a flow diagram showing steps of an example computer-implemented method for performing a blockchain based transaction.

FIG. 27 performing a smart contract transaction between entities

FIG. 28 illustrates a high-level block diagram of an example, non-limiting system that can facilitate generating avatar non-fungible tokens (NFTs) and applications related to usage thereof in accordance with one or more embodiments described herein.

FIG. 29 presents an example avatar platform that facilitates generating avatar NFTs in accordance with one or more embodiments.

FIG. 30 presents an example computer-implemented method for generating avatar NFTs in accordance with one or more embodiments described herein.

FIG. 31 presents another example computer-implemented method for generating avatar NFTs in accordance with one or more embodiments described herein.

FIG. 32 presents another example avatar platform that facilitates extended reality applications using avatars in accordance with one or more embodiments;

FIG. 33 presents an example computer-implemented method for employing other user's avatars in association with XR gaming applications, in accordance with one or more embodiments described herein.

FIG. 34 illustrates an example computer-implemented method for demonstrating usage of equipment using an avatar, in accordance with one or more embodiments described herein.

FIG. 35 is a schematic block diagram illustrating a suitable operating environment in accordance with various aspects and embodiments.

FIG. 36 is a schematic block diagram of a sample-computing environment in accordance with various aspects and embodiments.

### DETAILED DESCRIPTION

### I—OVERVIEW

The innovation is described with reference to the drawings, wherein like reference numerals are used to refer to like elements throughout. In the following description, for purposes of explanation, numerous specific details are set forth in order to provide a thorough understanding of this innovation. It may be evident, however, that the innovation can be practiced without these specific details. In other instances, well-known structures and components are shown in block diagram form in order to facilitate describing the innovation.

By way of introduction, the subject matter described in this disclosure relates to manifesting responses to physical and physiological activity information and image data about a user using a virtual avatar presented to the user. In computing technology, an avatar is a graphical representation of a character or personality provided to convey information to a viewer. An avatar can take a two or three-dimensional human form and be configured to speak and move as would a real person. The disclosed subject matter relates generally to using an avatar whose behavior and/or appearance changes based in part on received physical and physiological activity information and image data about a user. The physical and physiological activity information can include sensed physiological or biometric information about the user, motion data regarding movement of the user and image data corresponding to two or three-dimensional image captures of the user. In an aspect, the physical and physiological activity information is captured via one or more biometric or motion sensors attached to the user. In another aspect, the physical and physiological activity data is captured via an image capture device (e.g., a camera, a video camera, a three-dimensional image capture device, a three-dimensional scanner, etc.). In another aspect, the physical and physiological activity data is captured via a remote sensing device employed by the user (e.g., an external medical monitoring device, a handheld sensing device, a handheld remote, a smart scale, a balance pad, fitness device employed by the user, etc.).

For example, depending on the types of biometric sensors employed, collected physiological data about a user can include but is not limited to: heart rate, body temperature, respiration, perspiration, blood pressure, calories burned, body fat, and body weight. When biosensors are employed, other physiological data based on chemical body reactions can be determined, such as glucose levels, cortisol levels, blood alcohol levels, presence of drug residues, presence of pathogens, presence of bacteria, etc. In addition, depending on the number and sophistication of motion sensors employed, collected movement data can range from the detection of general movement pattern-based motions such as walking, running, sitting, jumping, climbing, etc., to even minute bodily motions such as strokes of a finger or the blinking of an eye. Further, image data can be captured of a user to facilitate generation on two or three-dimensional representations of the user.

In accordance with an embodiment, an avatar guidance system is provided that analyzes received physical and physiological activity data about a user to evaluate adherence of the user to predefined routine or program. In response to analysis of received physical and physiological activity data for a user, visual changes and/or verbal commands for manifestation by an avatar presented to the user are determined and effectuated. In particular, an avatar presented to the user is configured to exhibit visual and/or audible behavioral reactions based on comparison of monitored data representing a metric of a user's physiology and/or physical movement with respect to one or more reference metrics associated with a routine or program the user has selected to follow. For example, in response to user physical and physiological activity data falling outside a predefined range for a specific user behavior associated with performance of an action, task or routine, in order to correct the behavior of the user, the avatar can react in a way designed to reflect model techniques and behavior.

Visual and verbal avatar reactions can embody those which a real human may perform and can include but are not limited to changes in: motion or movement, speech, tone of voice, level of sound/loudness, facial expressions, body language, color, speed of movement, and range of motion. In an aspect, responses to user physical and physiological activity data manifested by a virtual avatar as described herein are determined and effectuated in real-time (e.g., in response to receipt of the physical and physiological activity data). For example, a graphical user interface can be presented to the user that dynamically generates the avatar performing or exhibiting the determined responses. In another aspect, the avatar is rendered and presented to the user in the form of a three-dimensional hologram.

Existing systems, such as various gaming programs, have employed avatars to mimic user motion data. For example, in response to a sensed jumping motion by a user, an avatar presented to the user may perform the jumping motion. However, rather than simply mimicking user motion, in accordance with various aspect of the disclosed avatar guidance system, an avatar is configured to respond to a user based on evaluation of the user's physical and physiological activity data with respect to a personalized program developed for the user that the user strives to follow. In particular, the avatar is configured to function as an intelligent being that has been specifically trained to observe, analyze and respond to a user's physical and physiological activity data based on a set of parameters defining at least one of: how the user should and shouldn't move, how the user's physiology should and shouldn't function, what actions or activities the user should and shouldn't perform, when the user should and shouldn't perform certain actions or activities, or how the user should and shouldn't appear. This set of parameters can be specifically tailored to govern user behavior with respect to a predetermined action, task or program.

For example, when performing a fitness routine that is associated with known or model/reference movement and physiological parameters for performance of the fitness routine by a model user, information can be collected about the user regarding the user's movement and physiology. The user's collected information is then compared to the known movement and physiological parameters to determine whether the user is adhering to the routine from an anatomical physical movement perspective and a physiological perspective. In response to a determination that the user's physical and physiological activity data deviates or does not deviate from the known movement and physiological parameters, a response is determined that is capable of being manifested by an avatar presented to the user. This response can provide the user with guidance to facilitate adherence to the fitness routine.

For instance, a response can include a determined physical action for the user to perform to correct the deviation and this physical action can be demonstrated by the avatar. As one example, if a monitored user on a treadmill is slowing down when a routine followed by the user does not call for slowing down, an avatar configured to guide or coach the user can change behavior to demonstrate a faster pace, thereby encouraging the user to speed up. The avatar can also provide verbal instructions regarding performance of the physical action.

In another example, in response to a determination that the user's physical and physiological activity data deviates or does not deviate from the model or reference movement and physical parameters for the fitness routine, a response can be determined for manifestation by the avatar that provides criticism, motivation, praise, comparison, or further instruction. This response can encompass movement by the avatar, speech, facial expression, tone of voice, or any other real life physical human reaction that could be envisioned to facilitate expression of criticism, motivation, praise, etc. In this way, as a user performs a fitness routine, program, action or task, the user can feel as though a real person was observing the user in real-time and responding in the form of personal intelligent coach that is an expert in evaluating and guiding performance of the specific routine, program, action or task.

In addition to comparison of received user physical and physiological activity data to reference physical metrics for a specific routine, program, action or task, various aspects of the disclosed avatar guidance system provide for personalized analysis of user physical and physiological activity data and personalized responses based on the analysis for manifested by an avatar. In one aspect, the reference physical and physiological activity metrics for the specific routine, program, action or task that is monitored for a user can be tailored or calibrated to fit the user's personal physical capabilities, functions and goals. For example, in furtherance to the physical fitness routine example, metrics for a user's range of motion, motion speed, heart rate, blood pressure, etc., can be tailored and calibrated to fit the user's personal goals and account for the user's physical capabilities.

Further, responses to adherence or deviation from a routine, program, task or action (e.g., based on comparison to reference metrics or personally calibrated reference metrics) for manifestation by an avatar can be tailored based on user preferences, demographics, mood, language, location, or context. For example, with respect to an avatar that functions as a user's fitness trainer, an individual may provide preferences that note what type of exercises the user likes and doesn't like to perform or what type of coaching motivates the user (e.g., soft encouraging technique over a more pushy or demeaning approach). A response determined for the avatar to perform can then be tailored to account for the user's preferences. For instance, when the user is not achieving a particular exercise included in the fitness routine, the avatar can provide assurances that the user is “doing his best and that is all that matters,” as opposed to harping at the user and pushing the user “to get it right or get ten more minutes of abdominal work at the end of the session.” In another example, an avatar that is configured to provide instruction to a child can use different language and instructional techniques than an avatar configured to provide instruction to an adult. In yet another example, an avatar that is configured to provide guidance to a user throughout the day to adhere to a diet plan can suggest food options based on the user's context including the user's location, schedule and time of day.

Moreover, responses determined for manifestation by an avatar (based on received user physical and physiological activity data) to provide a user with guidance, instruction or support with respect to performing an action, program, task or routine can be based on learned user behavior. For example, historical data regarding past reactions/responses performed by the user to avatar responses in association with same or similar routines, tasks or actions can be collected and analyzed using various machine learning techniques to determine what types of avatar responses work and don't work for the user. In another example, traits, habits and abilities of the user can be learned over time and employed to tailor avatar responses. For instance, when suggesting a new exercise to transition to when a user is not achieving a previously suggested exercise, the avatar can select an exercise that the user is known to perform well to boost the user's spirit. In another example, when a user is known to stop at a fast-food restaurant on Elm street when experiencing high stress levels on the way home from work, an avatar designed to help the user adhere to a diet program can warn the user that the user is likely to perform this mistake and suggest an alternate route and food option for the user.

The subject techniques for providing a user with guidance, instruction or support with respect to performing an action, program, task or routine via a virtual avatar based in part on monitored user physical and physiological activity data can have a variety of applications. In one aspect, the disclosed techniques are tailored to facilitate guiding a user through a physical fitness routine or program, such as a personal training session, a physical therapy program or any other physical activity that may require an instructor, coach, teacher, therapist, trainer, etc. Some examples are ergonometrics, dancing, yoga, zumba, and martial arts. For fitness applications, an avatar can play the role of a virtual instructor, coach, teacher, physical therapist, or personal trainer. During performance of a fitness routine, a user's physiological and movement data is collected and compared to reference physical and physiological activity metrics know for the specific fitness routine in view of personal information for the user (e.g., health restrictions, preferences, goals, etc.) to determine whether an avatar response is warranted and if so, what the avatar response should be (e.g., a specific verbal and/or visual command). After an avatar response is determined, the avatar displayed to the user performs the response.

In another aspect, the disclosed techniques can facilitate guiding a user with adherence to a diet program, a health program, a stress reduction program, a schedule and virtually any type of program that can be monitored and adapted based at least in part on physiological characteristics/changes of the user and/or movement/motion of the user. Regardless of the application of the disclosed avatar guidance system, by employing a virtual avatar, instruction and information provided to guide or coach a user through a task or program is presented to the user by virtual human exhibiting real life human visual and audible responses in the manner in which a personalized coach, therapist, assistant, friend, expert, doctor, etc. with particular insight about the user would respond. Accordingly, the user will feel as though a real person was by his or her side every step of the way throughout the program or activity the user is striving to perform. In addition, this ‘real person’ embodied via an avatar will be an expert at evaluating not only adherence to a specific program at hand, but a personalized guide that knows just how to tailor evaluation of adherence to the specific program for the user and how to tailor responses based on the evaluation to facilitate the user's adherence to the specific program at hand.

In accordance with another embodiment, systems and techniques are disclosed that provide for visualizing physical changes in a user based on performance or predicted performance of a fitness or diet program. In particular, an avatar visualization system is provided that is configured to receive or generate a visual replica of a user as the user currently appears. This visual replica can be embodied as an avatar presented to the user. As the user's appearance changes over the course of performance of a health and fitness program, the appearance of the avatar can also change to provide a mirrored visualization of the changes in the user. For example, each new day of the program, the appearance of the user can be evaluated, captured and imparted to the avatar. Thus in essence, the user's avatar can resemble a mirror image of himself over the course of a program.

The avatar visualization system is further configured to generate a visual representation of the user that is a prediction of how the user will appear at a future point in time based on performance of a health and fitness program by the user. In particular, the avatar visualization system can generate a visual representation or replica (e.g., an avatar) of a user based on currently received appearance information for the user, currently received physical and physiological activity data for the user (e.g., physiological data and/or movement data), and known health information for the user (e.g., physical measurements, physical conditions, physical capabilities, etc.). For example, the user can select, (and/or design, or otherwise be assigned) a specific health and fitness program for performance. The user can further be provided with a visual representation that demonstrates how the user will predicatively look after performance/completion of the program if the user adheres to the requirements of the program. In addition, the user can select various time points in the program (e.g., after week 1, after week 2, after week 3, etc.) and the avatar visualization system can generate a visual representation of the user that demonstrates how the user will predicatively look at the respective time points if the user adheres to the requirements of the program.

This avatar visualization system allows a user to dynamically pick and choose different health and fitness programs and/or change different variables of a health and fitness program and see how the user would appear in the future based on the selected health and fitness program and/or the different variables. Accordingly, a user can select a health and fitness program that will cause the user to achieve an optimally desired appearance. For example, as the user selects different health and fitness programs and/or can changes variables of a selected health and fitness program, the avatar visualization system can dynamically adapt the appearance of an avatar presented to the user that corresponds to a predicted visualization of how the user will appear based on completion and adherence to the different health and fitness or the health and fitness program with the respectively chosen variables. As a result, the user can select a specific health and fitness program based on how it will affect the user's appearance.

### II—Example System Architecture

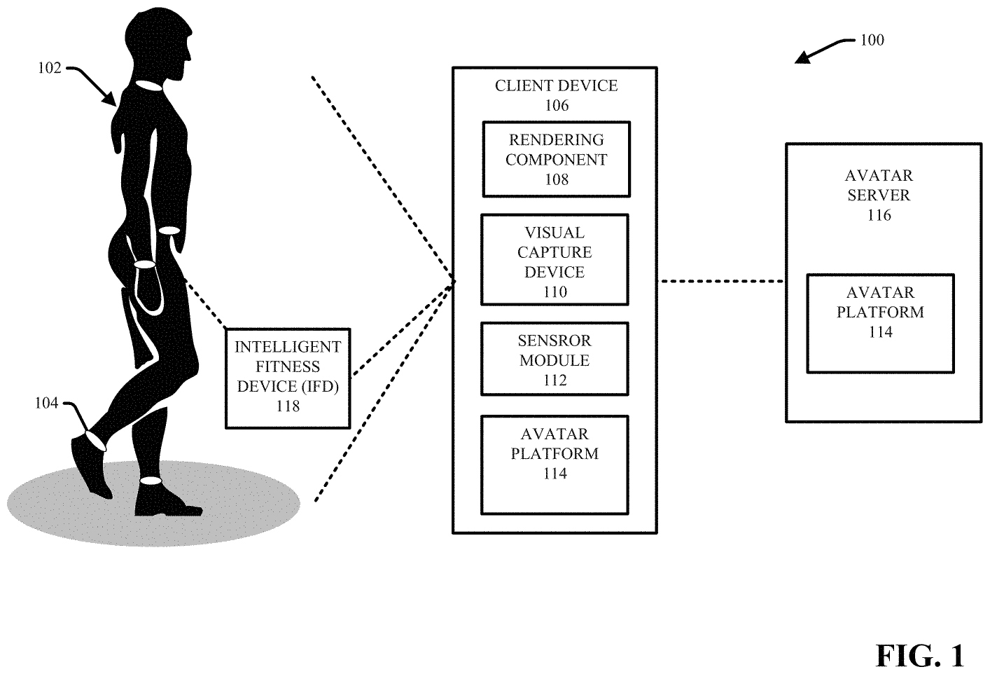

Referring now to the drawings, with reference initially to FIG. 1, presented is a diagram of an example architecture of a system 100 that facilitates manifesting responses to physical and physiological activity data about a user using a virtual avatar in accordance with various aspects and embodiments described herein. The architecture of system 100 can be employed in association with the disclosed avatar guidance system 200 discussed in Section III and the disclosed avatar visualization systems 1300 and 1400 discussed in Section IV and additional systems described herein. Aspects of systems, apparatuses or processes explained in this disclosure can constitute machine-executable components embodied within machine(s), e.g., embodied in one or more computer readable mediums (or media) associated with one or more machines. Such components, when executed by the one or more machines, e.g., computer(s), computing device(s), virtual machine(s), etc. can cause the machine(s) to perform the operations described.

System 100 includes a person/user 102 and a client device 106 configured to receive and/or capture physical and physiological activity data (e.g., physiological/biometric data, motion data, image data, etc.) about the user 102. In an aspect, one or more sensor devices 104 can be worn or otherwise attached to the user 102 to capture the physical and physiological activity data and transmit the captured physical and physiological activity data to client device 106 (e.g., in real-time or substantially real-time). For example, the one or more sensor devices 104 can include biometric sensors configured to detect information regarding at least one of: heart rate (e.g., via a heart monitor device), body temperature (e.g., via a thermometer), respiration, perspiration, blood pressure, electrical activity of the heart or brain, calories burned, or body fat and body weight. In another example, the one or more sensor devices 104 can include biosensors configured to detect information regarding at least one of: glucose level, cortisol level, blood oxygen level, blood alcohol level, inflammation, nitric oxide level, drug levels/residues present in the body, pathogens present in the body, or bacteria present in the body. In an aspect, the one or more sensor devices 104 can include implanted medical devices/sensor configured to detect and wirelessly communicate a wide array of biometric information about the user 102 to client device 106.

In addition to physiological/biometric data, sensor devices 104 can facilitate capture and reporting of user movement or motion corresponding to speed, direction, and orientation of the user a whole and/or individual body parts of the user. For example, the one or more sensor devices 104 can include motion sensors such as an accelerometer, a gyroscope or an inertial measurement unit (IMU). Thus, captured motion data can include information identifying acceleration, rotation/orientation, and/or velocity of the motion sensor device 104 itself, facilitating determination of motion and movement data of the body and/or body parts to which the motion sensor are attached. For example, processing of raw motion data (e.g., by the senor itself or the device to which the raw motion data is communicated, such as client device 106 and/or avatar server 116) can include pattern analysis to determine or infer types of motion represented by the motion data and/or characteristics associated with the types of motion (e.g., intensity, duration, range, speed, etc.). For example, using pattern recognition, patterns in the motion data can be correlated to know patterns for different types of motion, such as walking, running, climbing, jumping, falling, cycling, turning, etc. Motions sensors such as accelerometers can also be used in detection for Parkinson's disease. Patients of Parkinson's disease are known to have involuntary shaking. For those having mild symptom, shaking might not be significant. Since an accelerometer is sensitive enough to detect even mild shaking, when it is placed on a patient's arm while he/she is intended to hold the arm in still, the accelerometer could report involuntary shaking.

In another aspect, physical and physiological activity data regarding movements/motions performed by user 102 can include information captured by an intelligent fitness device (IFD), (hereinafter IFD 118) employed by the user in association with performance of a fitness routine or exercise. For example, some fitness exercises can involve usage of fitness equipment, such as exercise machines (e.g., a treadmill, a bicycle, a rowing machine, a weight machine, a balance board, etc.) or accessories (e.g., free weights, weighted balls, hula hoops, yoga blocks, bands, etc.). In an aspect, this fitness equipment can include one or more sensors configured to track and record user movements and motions and report (e.g., via wireless or wired connections) these movements and motions to client device 106 (and/or avatar server 116). For example, sensors and monitoring devices included in a treadmill can track user speed and incline. In another example, sensors included in various weight machines or free weights can detect and report number of reps, intensity, weight used, range of motion etc. Still in yet another example, a balance board or pressure sensitive mat can detect user movement performed while standing on the matt based on pressure and weight distribution. In another example, the IFD 118 can correspond to a smart scale configured to measure and report user weight. In another example, the IFD can correspond to a smart mirror configured to detect and report other physical measurement metrics for a user, such as but not limited to: height, weight, body mass index (BMI), specific body part measurements (e.g., waist, chest, upper arm/bicep, thigh, etc.), body fat, body composition, muscle composition, muscle density, and the like. In this regard, the IFD 118 can correspond to one or more of these types of intelligent fitness devices configured to detect and generate the corresponding health and/or fitness information described above (among others) and include communication technology (e.g., wireless and/or wired communication technology) to communicate the data to the client device 106 and/or the avatar server 116. Thus, in some respects, the IFD 118 can correspond to an Internet of Things (IoT) communication device configured to automatically detect/generate and communicate the corresponding health and fitness information for the user to another device of system 100.

In yet another aspect, physical and physiological activity data about a user corresponding to movement/motion and appearance of the user 102 can be captured by client device 106. For example, client device 106 can include a visual capture device 110 such as a still image camera, a video camera, or a three-dimensional camera/scanner configured to capture image and/or video data of the user 102. According to this example, client device 106 can collect video and still images of the user 102 as the user performs an activity, task, or routine (e.g., a workout routine). The image data can be analyzed using pattern recognition technology to determine whether the user's movement corresponds to model movement metrics for the activity, task or routine. For instance, while performing a fitness routine such as a yoga or dance routine, image data captured by visual capture device 110 can be processed and analyzed (e.g., in real-time) to determine whether the user is executing the correct movements/poses and using proper form.

In some embodiments, the image data can be processed by the client device 106 and/or the avatar server 116 using intelligent image analysis to automatically determine or infer physical measurement data for the user 102, such as but not limited to: height, weight, BMI, specific body part measurements (e.g., waist, chest, upper arm/bicep, thigh, etc.), body fat, body composition, muscle composition, muscle density, and the like. For example, image data for a user captured by visual capture device 110 can be analyzed using two dimensional and/or three-dimensional image analysis to determine physical measurements metrics for the users, such as height, weight, body fat, dimension of the user's waist, hips, shoulders, biceps, etc. As a user's body changes over time (e.g., in response to performance of an exercise or diet program), changes to physical measurement parameters of the user can be tracked, recorded, and monitored. In addition, a visual replica (e.g., a two-dimensional image or three-dimensional image/model) of the user as the user actually appears (e.g., standing still or during performance of a fitness routine), should appear (e.g., based on a model) or may predicatively appear in response to advancement in a fitness/diet program, can also be generated. In an aspect, an avatar that is generated and presented to the user (e.g., via rendering component 108 and/or avatar platform 114) can be modeled to replicate the user's physical appearance based on the captured image data.

In another aspect, client device 106 can include a sensor module 112 that can aid with detection of user motion and motion data using infrared or visible light detection devices. In yet another aspect, client device 106 can include a wearable device and sensor module 112 can include biometric and/or motion sensors configured to capture the various physical and physiological activity metrics described with respect to sensor devices 104.

Client device 106 also includes rendering component 108 to generate and present an avatar to the user 102. For example, rendering component 108 can be configured to generate a graphical user interface that includes the avatar and rendered via a display screen of the client device 106. In another example, rendering component 108 can be configured to generate an avatar as a hologram that is presented to the user 102. In an aspect, an avatar generated/presented via rendering component 108 perform various visual and/or audible actions based at least in part on analysis of the physical and physiological activity data as described herein. In another aspect, an avatar generate/presented to the user via rendering component is configured to provide a visual replica of the user.

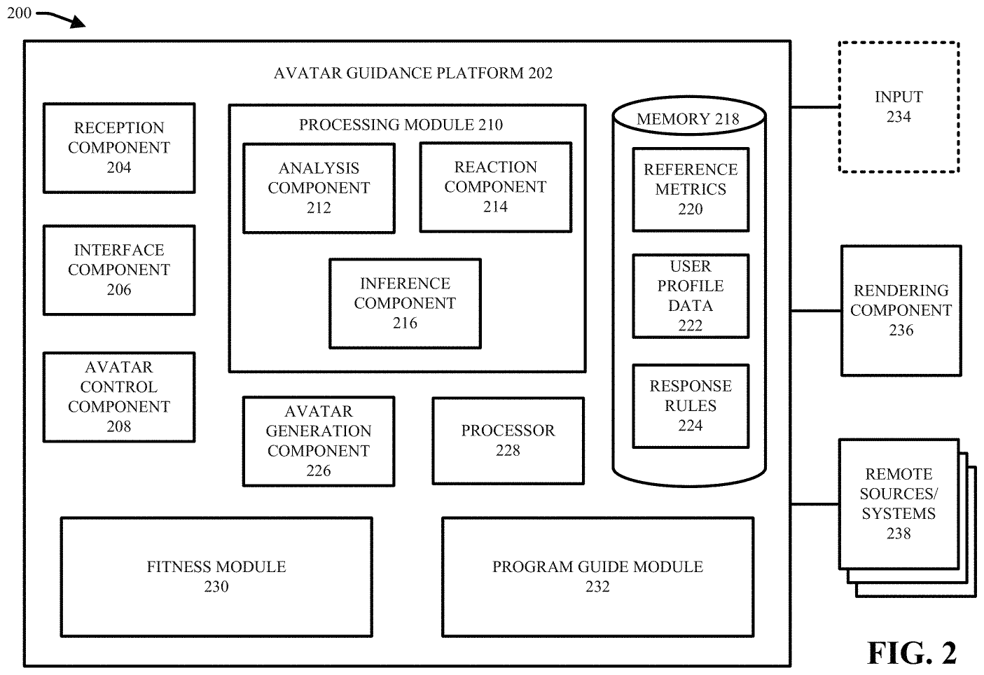

In an aspect, client device 106 can include avatar platform 114 to provide for processing and analyzing of user's physical and physiological activity data to facilitate determination and manifestation of avatar reactions and appearances to the data in accordance with aspects described herein. In another aspect, some or all of the processing and analysis of the physical and physiological activity data is performed by a remote avatar server 116. According to this aspect, avatar server 116 can include an avatar platform 114 and client device 106 can communicate received and/or captured physical and physiological activity data to the avatar server 116 for processing thereof. The avatar server 116 can further communicate control commands, determined based on the processed physical and physiological activity data, that control the various visual and/or audible actions of the avatar generated and displayed at client device 106 by rendering component 108. In another aspect, various processing and analysis functions associated with avatar platform 114 can be divided between client device 106 and avatar server 116. The various features and functions of avatar platform 114 are described in greater detail infra with respect to FIGS. 2, 13 and 14.

System 100 can include one or more networks to facilitate connection/communication between the one or more sensor devices 104, client device 106, and/or avatar server 114. These networks can include wired and wireless networks, including but not limited to, a personal area network (PAN), a local area network (LAN) a cellular network, or a wide area network (WAD, e.g., the Internet). For example, a sensor device 104 can communicate with client device 106 using a PAN (e.g., via short range radio communications such as Bluetooth™, near field communication (NFC), etc.). In another example, a sensor device 104 and/or client device 106 can communicate with avatar server 114 (and vice versa) using virtually any desired wired or wireless technology, including, for example, cellular, WAN, wireless fidelity (Wi-Fi), Wi-Max, WLAN, and etc. In addition, the one or more sensor devices 104, client device 106, and/or avatar server 114 can include memory that stores computer executable components and a processor that executes the computer executable components stored in the memory, examples of which can be found with reference to FIG. 99.

Client device 106 can include any suitable computing device that can facilitate generating and presenting an avatar to a user 102 that is reactive to user input including physical and physiological activity data generated or provided by the user 102. For example, client device 106 can include a desktop computer, a laptop computer, a television, an Internet enabled television, a mobile phone, a smartphone, a tablet personal computer (PC), a personal digital assistant PDA, or a wearable device. It should be appreciated that the size of client device 106 with respect to user 102 as drawn in FIG. 1 is not intended to provide a scaled relationship between one another. For instance, in an aspect, client device 106 can include a small device configured to be worn on the user's wrist (e.g., a smart watch), a handled device (e.g., a smartphone, a tablet PC, etc.) a device configured to be worn as a headband, glasses, or another form of eye-were or heads-up display device (e.g., an augmented reality (AR), virtual reality (VR), or extended reality (XR) device), or large display device presented to the user 102 (e.g., a television display, a smart-television, etc.). As used in this disclosure, the terms “content consumer” or “user” refer to any entity (e.g., person, group of people, animal, group of animals, machine, system, or combination thereof) that employs system 100 (or additional systems described in this disclosure) using a client device 106.

### III—Example Avatar Guidance System

In one or more aspects, an avatar guidance system is provided that includes a reception component configured to receive physical and physiological activity information about a user during performance of a routine or program, and an analysis component configured to analyze the physical and physiological activity information based on reference physical and physiological activity metrics for the routine or the program to determine whether, how and to what degree the user deviates from requirements of the routine or the program. The system further includes a reaction component configured to determine a response for an avatar displayed to the user based on a determination regarding whether, how and to what degree the user deviates from the requirements of the routine or the program, and an avatar control component configured to initiate manifestation of the response by the avatar as displayed to the user during the performance of the routine or the program.

In another aspect, a method is disclosed that includes receiving information about a user during performance of a physical routine, wherein the physical information includes physiological information and anatomical movement information. The method further includes determining whether and how the user deviates from requirements of the physical routine based on comparison of the information to reference physical and physiological activity metrics for the physical routine, and determining a response for an performance by avatar displayed to the user based on and in response to a determination regarding whether and how the user deviates from the requirements of the physical routine, wherein the response is configured to facilitate adherence to the requirements of the physical routine. The method further includes initiating manifestation of the response, in response to the determination thereof, by the avatar as displayed to the user during the performance of the physical routine.

Still in yet another aspect, a system is provided that includes an interface component configured to generate a graphical user interface comprising an avatar configured to react to a user during performance of a physical routine by the user in response to a determination that the user deviates from a physical requirement of the routine based on analysis of physical and physiological activity data about the user generated during performance of the physical routine. The system further includes an avatar generation component configured to cause the avatar to perform a reaction that provides instruction to correct the manner in which the user deviates from the physical routine in response to reception of control commands defining the reaction, wherein the reaction comprises at least one of speech or movement; and a rendering component configured display the graphical user interface with the avatar performing the reaction.

FIG. 2 presents an example avatar guidance system 200 that facilitates guiding or assisting a user with adhering to a program, routine or activity using an avatar in accordance with various aspects and embodiments described herein. System 200 can include same or similar features and functionalities as system 100. In an aspect, system 200 employs the architecture of system 100, including one or more of the various components/devices of system 100, wherein avatar guidance platform 202 corresponds to avatar platform 114. Avatar guidance platform 202 can thus include same or similar features and functionalities as avatar platform 114. Repetitive description of like elements employed in respective embodiments of systems and interfaces described herein are omitted for sake of brevity.

Avatar guidance system 200 can include avatar guidance platform 202, input 234, rendering component 236 and one or more remote sources/systems 238. Avatar guidance platform 202 is configured to facilitate guiding or assisting a user with adhering to a program, routine or activity using an avatar that is responsive to various input 234 associated with performance of the program, routine or activity. This input 234 can include physiological, motion and/or image data about the user received (e.g., in real-time) during performance of the program, routine or activity.

For example, avatar guidance platform 202 can receive physical and physiological activity data for a user during performance of a fitness routine and compare this physical and physiological activity data to reference physical and physiological activity data for the fitness routine to determine whether the physical and physiological activity data deviates from the reference physical and physiological activity data. In response to a determination that the user is moving too fast or too slow, has a heart rate to high or too low, is incorrectly or correctly performing a fitness movement, etc., avatar guidance platform 202 can determine a response to manifest via an avatar to respond to the deviation or non-deviation. For example, avatar guidance platform 202 can determine an appropriate verbal command or remark for the avatar to speak (e.g., “your heart rate is too low, lets speed it up”) and/or a physical appearance/motion for the avatar to effectuate (e.g., the avatar can demonstrate the correct motion, the avatar can express an emotion of gratitude via a facial expressions and body movements, etc.). Avatar guidance platform 202 can then cause an avatar generated and presented to the user via a GUI (or as a hologram) to carry out the response (e.g., speak the command, perform the motion and appearance change, etc.).

In an aspect, the avatar is generated and presented to the user via a rendering component 236 located at a client device (e.g., client device 106). Rendering component 236 can include same or similar features and functionality as rendering component 108. Rendering component 236 can include suitable hardware (e.g., a display screen, a hologram generation device, etc.) and software (e.g., software for generating a GUI and/or software for accessing and rendering network based interface, such a browser) to accomplish generating and presenting an avatar that performs the responses determined by avatar guidance platform 202 to facilitate guiding the user with adhering to the program, routine or activity being monitored.

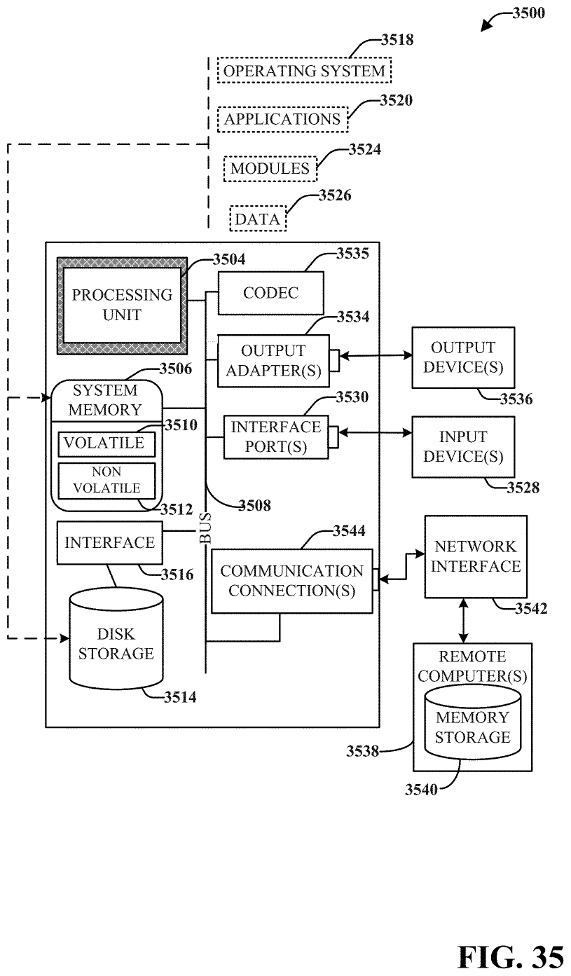

Generally, avatar guidance platform 202 can include memory 218 that stores computer executable components and processor 228 that executes the computer executable components stored in the memory, examples of which can be found with reference to FIG. 35. It is to be appreciated that although avatar guidance platform 202 is illustrated as being a standalone component, such implementation is not so limited. For example, avatar guidance platform 202 can be located at a client device (e.g., client device 106), a remote server (e.g., avatar server 116) or the cloud. In another aspect, one or more components of avatar guidance platform 202 can be located at both the client device and the remote server. Still in yet another aspect, the components of avatar guidance platform can be distributed between the client and the server.

In addition to physical and physiological activity information and/or image data about a user, input 234 can also include profile information for the user that defines various known characteristics of the user, including but not limited to, health information, preferences, demographics, user schedule, and historical information gathered about the user over the course of a monitored program, routine or activity. This input can also include contextual information associated performance of the program, routine or activity, such as a location of the user, information about the location (e.g., a map of the location, physical structures at the location, events occurring at the location, etc.), weather information, information about other persons the user is located near and/or interacting with, and other information provided by the user during performance of the program, routine or activity (e.g., information about the user's current mood or information about what the user is currently thinking/feeling).

Avatar guidance platform 202 is configured to receive and process these various inputs 234 about a user's physical state and activity along with various other personal and contextual information to facilitate guiding and assisting the user with performance of a specific program, routine or task via manifestation of responses based on the input to the user through an avatar. In particular, the avatar is configured to function as an intelligent being that has been specifically trained to observe, analyze and respond to a user's physical and physiological activity data, profile information and context information in association with performance of a specific program, routine or task, based on various rule based classification schemes for the program, routine, or task, defining at least one of but not limited to: what actions or activities the user should and shouldn't perform, how the user should and shouldn't move during performance of the actions or activities, how the user's physiology should and shouldn't function during performance of the actions or activities, when the user should and shouldn't perform certain actions or activities, where the user should and shouldn't perform certain actions or activities, or how the user should and shouldn't appear. In some aspects, avatar guidance platform 202 can even monitor interaction and association of the user with other people with respect to performance of actions and activities of defined in a program, routine or task. For example, avatar guidance platform 202 can receive information indicating the user is located within X feet of an identified person and determine whether the user is authorized to interact with the identified person (e.g., at all or under the current context) based on a defined program the user is following.

Based on analysis of received input 234 with respect to a defined program, routine or task the user is performing, reactions are determined for manifestation by an avatar presented to the user. These reactions can include visual and/or audible (e.g., speech responses) responses that provide instruction, guidance, motivation, and evaluation for the user with respect to the user's performance (or non-performance) of the program, routine or task. The visual and verbal avatar reactions can embody those which a real human may perform to provide the instruction, guidance, motivation, etc. and can include but are not limited to changes in: motion or movement, speech, tone of voice, level of sound/loudness, facial expressions, body language, color, speed of movement, and range of motion. In an aspect, possible reactions for performance by an avatar in association with a specific program, routine or activity are predefined and stored in memory 218. In another aspect, reactions for performance by an avatar can be dynamically determined based on a set of response rules for the specific program, routine or activity. These response rules can account for user physical and physiological activity data as well as user profile information and user context information. In an aspect, avatar guidance platform 202 can employ various machine learning techniques to infer a suitable reaction for an avatar based on the various inputs and information accessible to avatar guidance platform 202.

In an aspect, a suitable routine or activity capable of being monitored by avatar guidance platform 202 can involve one where a physical trainer, teacher or demonstrator could potentially provide the user assistance/guidance with performing the routine or activity. For example, the routine or activity can include a physical exercise or routine where a coach or trainer may provide instruction and/or evaluation, such as performance of a physical therapy session, a planned workout, an aerobics routine, a dance routine, a yoga routine, track and field training, or a sports activity (e.g., football, baseball, soccer, basketball, tennis, golf, fencing, gymnastics, skiing, karate, horseback riding, juggling, rock climbing, diving, etc.). In another example, the routine or activity can include any activity that involves some form of physical movement that could be demonstrated and evaluated by another person, such as cooking a dish, building something, creating a piece of artwork (e.g., painting, sculpting), performing a medical operation, playing an instrument, etc.

A suitable a program for evaluation by avatar guidance platform 202 can include one in which the user is required to perform various physical or physiological actions over a period of time to achieve a goal. The period of time can vary. For example, the period of time could include a course of a few hours, a few days, a few weeks, a few months, etc. In an aspect, such a program could include a diet program, a fitness program, or a health program that involves monitoring and guiding a user with adherence to the program and tracking progress throughout the program based in part on physical and physiological activity input data for the user. For example, an avatar configured to guide a user through a program can function as the user's personal coach, trainer, conscience, doctor, therapist, advisor, friend, family member etc., or any other suitable persona, that can function to guide or coach the user through the program. In an aspect, this avatar can essentially know what the user is doing at all times with respect to the program (e.g., throughout the day, week, month, etc., or whenever system 200 is activated and accessible to the user).

For example, with respect to a diet/fitness program, as user physical and physiological activity input and/or image data is received, an avatar can respond to physical changes in the user, monitor what the user eats or how many calories the user consumes/burns, instruct the user what to eat and when, monitor when the user exercises, determine and instruct what exercises the user should perform, and assist the user with making intelligent choices in line with the diet/exercise program throughout the day. In another example, an avatar can function as a health guide for a user suffering from an illness or ailment, determining when the user should and shouldn't perform certain activities, when the user should take certain medications, when the user should seek emergency health services, when the user is becoming fatigued or sick, when the user's health is improving, etc., and responding accordingly with visual and/or audible reactions. In another example, an avatar can assist a user with overcoming an addiction, such as a drug addiction or alcohol addiction, by monitoring the user's physical state and activity throughout the day and providing the user with guidance and reactions in line with adhering to a rehab program. Still in yet another example, a user can set up a personal schedule that can involve activities the user should perform and when and avatar guidance platform 202 can monitor and facilitate adherence to the schedule.

Based on received inputs 234 for a user, (e.g., physiological data, motion data, image data, profile data, and/or context data) determinations and inferences can be made regarding adherence of the user to the program, routine or activity and mechanisms to facilitate adherence of the program, routine or activity. These mechanisms can be manifested by an avatar presented to the user at the time of need. For example, when a user is becoming unproductive at his work desk and fatigued based on input indicating the user's location, the user's motion activity, the user's brain activity, the user's glucose levels, and the user's cortisol level, the avatar can notify the user and suggest that the user get up and take a walk and eat a snack.

Reception component 204 is configured to receive input 234. Physical and physiological activity input 234 about a user can be received automatically from various sources including but not limited to: one or more sensors attached to the user (e.g., sensors 104), an intelligent fitness device employed by the user (e.g.,), and/or an external device employed by the user, such as a visual capture device (e.g., visual capture device 110), a sensor module (e.g., sensor module 112), or other physical data capture devices. In an aspect, raw physical and physiological activity data and/or image data is received by reception component as it is captured. For example, sensors (e.g., sensors 104) worn by the user can send physical and physiological activity data directed to reception component 204 in response to sensing thereby. Accordingly, physical and physiological activity data about the user can be received and processed in real-time or substantially real-time.

In addition to physical and physiological activity input captured by various external devices reception component 204 can receive some user profile and context information from the user or another user associated with the user (e.g., the user's physical therapist, the user's dietician, the user's mom, etc.) directly. For example, prior to beginning a particular program, routine, or activity, the user or the other user can provide avatar guidance platform 202 (e.g., via text or speech input) with profile information, such as information describing the user's health history, the user's preferences, the user's demographics, etc. In another example, during performance of the program, routine or activity, the user can provide avatar guidance platform 202 with feedback and/or update his or her profile information.

In another aspect, reception component 204 can extract some forms of input or information regarding a user's profile and the user's context from one or more remote sources 238 accessible to avatar guidance platform via a network (e.g., the Internet). For example, reception component 204 can access and import health information for the user from the user's health care provider. In another example, reception component 204 can gather a variety of information about the user's preferences, demographics, social affiliations, media preferences, etc. from various network sources the user accesses or employs (e.g., social networking sources, websites visited by the user, applications downloaded and employed by the user, articles and media accessed by the user, etc.).

In addition, reception component 204 can receive information regarding a user's location using various known location determination techniques. For example, the user can wear a locating device (e.g., a location tracking device, a global positioning system device, etc.) or a mobile client device employed by the user can include systems for determining a location of the mobile client. Location information can further be reported by these devices to reception component 204. Information regarding positions of other known persons relative to the user can be gathered in a similar manner (e.g., when such persons have authorized and enabled tracking of their location). In an aspect, reception component can receive information related to a user's location and/or environment (e.g., physical structures, information about the physical structures, activities in the environment, weather information, etc.) from various remote sources and/systems accessible to reception component 204 (e.g., map services, new services, merchant websites, etc.).

Processing module 210 is configured to process various input data 234 received to facilitate guiding and assisting the user with performance of a specific program, routine or task via manifestation of responses based on the input to the user through an avatar. Based on analysis of received input 234 with respect to a defined program, routine or task the user is performing, processing module 210 determines or infers reactions for manifestation by an avatar presented to the user. In an aspect, processing module 204 process user physical and physiological activity and context information as it is received and in view of user profile information to determine real-time responses to user actions (or non-actions) during a program, routine, or activity.

Processing module 210 includes can include analysis component 212, reaction component 214 and inference component 216. In an aspect, analysis component 212 is configured to analyze raw physical and physiological activity data for a user to determine or infer feature values corresponding to what the raw physical and physiological activity data represents. This analysis can occur in real-time or substantially real-time (e.g., as the physical and physiological activity data is generated and received, such as by client device 106 and/or avatar server 116). As described above, this raw physical and physiological activity data can include biometric data, motion or movement data, and image data. For example, biometric data could include information corresponding to a user's heart rate, blood pressure, blood oxygen level, temperature, glucose level, cortisol level, blood alcohol level, etc. In an aspect, received biometric data is received in a processed form to indicate the feature value it represents (e.g., heart rate, blood pressure, blood oxygen level, temperature, glucose level, cortisol level, blood alcohol level, etc.). In some aspects however, analysis component 212 can interpret raw signals from various sensors to determine feature values for biometric data.

Analysis component 212 can analyze raw motion data to determine a type of movement performed (e.g., walking, squatting, jumping, etc.) by the user and various characteristics of the movement, (e.g., range of motion, balance, weight/pressure, and even specific two or three-dimensional coordinate positions of respective parts of the user's body as they move as a function of time). For example, based on the motion data for the user as a whole and/or individual body parts of the user (e.g., corresponding to velocity, direction, orientation, position or pressure, etc.), analysis component 212 can determine a movement a user is performing including the precise position of the user's body parts with respect to one another and the ground or an apparatus over a period of time. In another example, analysis component 212 can determine feature values based on image data corresponding to the user's appearance, including two and three-dimensional measurements of the user's body and individual parts (e.g., while moving over time or at a still position), color data, characteristics of facial expressions, etc.

In addition to processing of received raw motion data, analysis component 212 can further determine a user's current context at a time during receipt (or non-receipt) of the physical and physiological activity data. For example, based on received context information, analysis component 212 can determine a location of the user at respective times throughout the day (e.g., using an internal clock). Analysis component 212 can further determine information about the location by parsing various remote sources/systems 238 that provide information related to the location and/or the current time. For example, analysis component 212 can extract a map of the location, determine what physical structures are at the location (e.g., houses, places of business, landmarks, etc.), information about the physical structures (e.g., hours of operation), events occurring at the location (e.g., traffic, sports match, etc.), current and prospected weather conditions of the location, etc. Other context data that can be provided to analysis component 212 and/or determined by analysis component 212 can include but is not limited to: information regarding other persons the user is located near and/or interacting with, what mood the user is in, or what the user is currently thinking/feeling. The various features that make up a user's context are referred to herein as context parameters.

Accordingly, analysis component 212 can essentially determine at any point in real-time at which input 234 is received, feature values corresponding to at least one of: a physiological state of the user, a movement of the user or characteristic of the movement, an appearance of the user, and a context of the user. Therefore, analysis component 212 can determine or infer what actions or activities the user is performing, how the is moving during performance of the actions or activities, how the user's physiology is functioning during performance of the actions or activities, when the user is performing the actions or activities, where the user is performing the actions or activities, how the user appears when performing the actions or activities, who is near the user during performance of the actions or activities, and what the user is thinking or feeling (e.g. based on information provided by the user and/or various inferences based on the other information and determinations) during performance of the actions or activities.

After analysis component 212 has determined feature values and/or context parameters based on received physical and physiological activity data and/or context data, respectively, analysis component 212 then analyzes adherence of the user's performance to a specific program, routine, or activity using these feature values and/or context parameters. The specific program, routine or activity that avatar guidance platform 202 is monitoring can vary as discussed above. In an aspect, the program, routine, or activity that is monitored by avatar guidance platform 202 is selected by the user (or another entity) from a list of predefined programs, routines, or activities that avatar guidance platform 202 is configured to monitor. In another aspect, avatar guidance platform 202 can allow the user or another entity to design a custom program, routine or activity for the user. In an aspect, custom programs, routines or activities can be saved and made available to other users of avatar guidance platform 202 for selection and performance.

For example, a user can select a specific activity or program (e.g., a fitness routine, a sports activity, a diet program, a stress reduction program, etc.) to be monitored by avatar guidance platform 202 or another user can elect the activity or program for the user (e.g., the user's real-life supervisor, doctor, coach etc.). For instance, the user can select a yoga routine, a golf lesson, a diet program, or a stress management program to follow and have monitored by avatar guidance platform 202. In another example, a user or supervisor of the user can create a custom program for a user that defines a schedule the user should adhere to, what actions the user should and shouldn't do and when, what physiological status the user should have and when, etc. In another aspect, a user can employ avatar guidance platform 202 to monitor several different programs, routines or activities for the user concurrently. For example, the user can employ the avatar guidance system 200 to set up a personal training avatar to monitor an exercise and fitness program for the user as well as a personal assistant avatar configured to monitor the user's adherence to a personal weekly schedule designed by the user.

In an aspect, based on known requirements for a program, routine or activity being monitored, analysis component 212 can determine or infer whether, how and to what degree the user is deviating from the program, routine or activity. For example, based on a user's physical and physiological activity feature values and/or context parameters and known requirements for these values/parameters for the particular program, routine or activity, analysis component 212 can determine or infer information including but not limited to, whether, how and to what degree: the user's actions or activities deviate from the program, routine or activity; the user's movements deviate from the program, etc.; the user's physiological state deviates from the program, etc.; and the user's appearance deviates from the program, etc. In another aspect, based on known requirements for a program, routine or activity (and user profile information), analysis component 212 can determine or infer whether, how and to what degree a user is likely to deviate from the requirements of a monitored program, routine or activity in the foreseeable future. For example, analysis component 212 can determine or infer when one or more physical and physiological activity feature values and/or context parameters are near or approaching (e.g., within a threshold degree of deviation) a limit value for the physical and physiological activity feature values and context metrics required by the program, routine, or activity (e.g., when your blood pressure is close to exceeding the limit, when your location is near a forbidden location and you are moving in the direction to the forbidden location, etc.).

In an aspect, known requirements for a specific program, routine, or activity can include predetermined reference metrics for the specific program, routine or activity defining values or value ranges for the what the user's physical and physiological activity feature values and/or context parameters should be. In an aspect, these reference metrics 220 are stored in memory 218. These reference metrics can be applied to various algorithms and classification schemes that relate the reference metrics to determined feature values and/or context parameters with respect adherence to of the user to a specific program, routine, or activity. Accordingly, analysis of physical and physiological activity feature values and/or context parameters with respect to reference metrics can involve various ruled based classification schemes wherein the feature values are analyzed based in part on the reference metrics for the program, routine or activity to determine whether the user's physical and physiological activity feature values and/or context parameters indicate the user is deviating from and/or likely to deviate from, the program, routine, or activity, how the user is deviating and/or is likely to deviate, and or to what degree the user is deviating and/or likely to deviate, (wherein likely can be measured in terms of probability).

In an aspect, evaluation of adherence to a specific program, routine or task includes a simple comparison of physical and physiological activity feature values and/or context parameters to known reference metrics for the specific program, routine or activity. In another aspect, the set of predefined reference metrics for a program, routine, or activity can include model/reference value ranges for the physical and physiological activity feature values and/or the context parameters. According to this, aspect, adherence and/or non-adherence to an aspect of a program, routine or activity can be based on deviation outside of a threshold range. A set of reference metrics for a program, routine, or activity can also include model/reference values or model/reference value ranges for different combinations of physical and physiological activity feature values and/or context parameters.

For example, based on a comparison of a physical and physiological activity feature value or set of physical and physiological activity feature values to a reference physical and physiological activity feature value or set of reference physical and physiological activity feature values, analysis component 212 can determine, whether the user's blood pressure is too high or too low, whether the user's body position is correct, whether the user's performing an exercise correctly with the proper intensity and form. In another example, based on such a comparison, analysis component 212 can determine, whether the user's stress levels are appropriate, whether the user's blood sugar is high or low, whether the user has consumed enough calories, whether the user has burned enough calories, or whether the user lost the desired amount of weight or inches of her waist, etc.

In another example, with respect to a fitness routine, reference metrics can define what the user's body movement and position should correspond to over the course of the fitness routine as well as what the user's heart rate should be with respect to each movement. In another example, with respect to stress reduction program, reference metrics can define what the user's heart rate, cortisol level, glucose levels, and activity levels and sleep patterns should correspond to throughout the day, when at various locations, when interacting with certain people, and/or with respect to certain activities, (e.g., working, driving, operating a certain machine, reading a book, going for a walk, etc.).

In another example related to analysis of physical and physiological activity feature values, based on comparison of biometric data for a user indicating a user's heart rate is 180 beats per minute (bpm) to reference metric data that defines the target heart rate for the exercise to be 150 bpm, analysis at step 210 can output a determination that the user's heart rate is too high, specifically 30 bmp too high. In yet another example, based on comparison of motion data indicating a user's motion pattern corresponds to X when is should correspond to Y, analysis at step 210 can output a determination that the user's motion is incorrect and even more specifically how the motion is incorrect (e.g., the user's legs should be bent another 10°).

In addition to physical and physiological activity feature-based reference metrics, programs, routines and be associated with context reference parameters that define requirements such as location, time of day, environmental characteristics, user mood, and other people within proximity to or interacting with the user. For example, a particular monitored activity or program, such as program designed to restrict an adolescent's social behavior (e.g., by the adolescent's parents), can be associated with various predetermined metrics and rule-based classification schemes regarding what the adolescent should be doing, when the adolescent should be doing it, where the adolescent should be located, and/or who the adolescent should or shouldn't be with. Accordingly, received contextual information regarding where a user is located and when, combined with external accessible information about the user's environment (e.g., physical structures, events, etc.) and received physical and physiological activity data for the user (e.g., movement patterns, the user's stress levels, the user's activity levels, user fatigue levels), can facilitate determining or inferring adherence to the activity or program.

For example, based on a user's location and time of day determinations can be made regarding the physical environment of the user, such as the terrain, what places of business are around, and what events are occurring at the location, (e.g., a football game). Depending on the user activity or program being monitored, this information can facilitate determinations regarding adherence to the activity or program. For example, an alcohol addiction recover program can include context metrics that define triggering locations for the user at specific, such as areas within X meters from a bar between the hours of 5 μm and 7 pm. According to this example, analysis component can determine or infer that the user is deviating from the program or is likely (e.g., has a determined probability of deviated over a threshold probability) when headed into an area with a bar a at happy hour time.

It should be appreciated that the above example comparison-based evaluations of physical and physiological activity feature values and/or context parameters to reference metrics are merely exemplary. In particular, a variety of more complex rule-based classification schemes can be defined that relate a plurality of concurrently received or applicable physical and physiological activity feature values and/or context parameters to one another and their respective reference values for the particular activity or program to determine whether, how, and to what degree, a user is deviating from a program, routine or activity.

In addition to comparison of determined physical and physiological activity feature values and/or context parameters to the reference metrics for the program, routine or activity, analysis component 212 can employ user profile data 222 to personalize evaluation of the user's performance and adherence to the program, routine or activity. As a result, the various aspects and applications of avatar guidance platform 202 can be highly personalized.

For example, in one an aspect, the set of predefined reference metrics for a specific program, routine, or activity can generically suited for a model user. In another aspect, the reference metrics can be provided or adapted by the user performing the activity or program or a supervisor of the user (e.g., an entity or person who instructed the user to perform the activity or program using the subject avatar response system 114, such as the user's coach, health advisor, rehab program administrator, mother, etc.). Still in yet another aspect, analysis component 212 can adjust or calibrate evaluation (e.g., using rule-based classification schemes and other intrinsic calculations) of physical and physiological activity feature values and/or context parameters using respective reference metrics for the program, routine or activity, based on user profile data.

User profile data 222 can include but is not limited to, health information, user preferences, user demographics, user schedule and historical information about the user's behavior in association with performance of various activities and programs monitored by the avatar guidance platform 202. Health information can include any information related to the user's health and physical state, including known physical metrics for the user (e.g., height, weight, body fat, etc.), physical conditions or ailments of the user, physical capabilities of the user, and any medications taken by the user. User preference information can vary depending on the application of the avatar guidance platform 202 for the user. Some examples of user preference information can include features and aspects of a fitness, health or other type of an activity or program that the user prefers/likes and/or does not prefer (e.g., foods the users likes/dislikes, dietary restrictions, places the user likes to go/not go, exercises the user likes/dislikes to perform, when the user liked to perform or not perform various activities, etc.). In another example, preference information can relate to what types of character traits that the user desires in an avatar designed to respond to, coach or instruct the user. Still in yet another example, user preference information can include information defining goals of the user (e.g., fitness goals, weight loss/weight gain goals, what the user desires to look like, milestones in a recovery program, and targets for performance of various other activities).

User demographic information can define various demographic features of the user, including but not limited to: gender, age, nationality, language, education level, or profession. User schedule information can provide information regarding what the user should and shouldn't be doing and when based on the user's work, extracurricular activities and personal relationships and responsibilities. For example, user schedule information can be provided to determine when a user should be at work and where, what meetings the user has, when the user has set up events or social activities, when the user has vacations scheduled, doctor appointments scheduled, physical therapy or fitness session scheduled, etc. In an aspect, user schedule information can be extracted from a personal calendar of the user (e.g., from an application employed by the user on the user's client device 106 and/or at remote source/system 238).

Profile information can be received from various sources. In an aspect profile information can be provided by the user in association with registration with avatar system (e.g., establishing a user account therewith), provided by another entity or source (e.g., medical professional, coach, fitness trainer, therapist, rehab program director), automatically imported by the avatar guidance platform 202 from various external sources (e.g., heath records, the user's calendar, social networking profiles, etc.), and/or automatically inferred or determined by the avatar response system.

In an aspect, analysis component 212 can employ user profile information to facilitate determining or inferring a probability at which a user is likely to deviate from a monitored program, routine or activity within the near future (e.g., within the next 10 seconds, within the next minute, within the next 10 minutes, within the next 30 minutes, within the next hour, etc.) based on physical and physiological activity features values and/or context metrics received/determined for the user. For example, analysis component 212 can analyze user patterns with respect to past deviations for program requirements similar to a current program requirement to determine the probability that the user will deviate from the current program requirement when physical and physiological activity feature values and/or context parameters for the user are x, y, and z, (where x, y and z correspond to variable values). In another example, analysis component 212 can analyze user preferences (e.g., what the user prefers to eat/drink, where the user prefers to go, what the user prefers to do, who the user prefers to associated with, etc.), to determine or infer how a degree of likelihood that the user will deviate from requirements of a monitored program given known information for the user's physical state (e.g., appearance, physiology), the user's body movement/position, and the user's context.

Reaction component 214 is configured to determine or infer a reaction (or non-reaction) for manifestation by an avatar presented to the user based on a determination by analysis component 212 identifying whether, how and to what degree a user is violating or is likely to violate a monitored program, routine or activity. The reaction is designed to keep the user's behavior and/or performance in line with the monitored program, routine or activity. For example, when a deviation is determined, reaction component 214 can determine a reaction for manifestation via an avatar that will instruct the user how to correct the deviation and/or encourage the user to correct the deviation or improve upon the deviation (e.g., where the deviation reflects positive behavior). In another example, when analysis component 212 determines that a user is complying with a program, routine, or activity, reaction component 214 can determine a reaction for manifestation via an avatar that provides praise to the user. In another example, when analysis component 212 determines that a user is likely to deviate from a program, routine or activity, reactions component 214 can determine a reaction for manifestation by an avatar that is designed to deter or prevent the user from deviated from the program, routine or activity.

In an aspect, a reaction determined by reaction component 214 for manifestation by an avatar can involve visual or spoken instruction by the avatar providing a mechanism to correct a deviation to a program, routine, or activity, an action taken by the avatar to deter or prevent the user from deviating from the program, etc., a tactic to motivate the user to adhere to the program, etc., a tactic to praise the user for adhering to the program, etc., or a tactic to criticize the user for not adhering to the program, etc. These reactions can involve visual and verbal actions which a real human may perform and can include but are not limited to changes in: motion or movement, speech, tone of voice, level of sound/loudness, facial expressions, body language, color, speed of movement, and range of motion. For example, the avatar can speak to the user and demonstrate body movement and facial expressions that a real human would in order to convey a reaction. In another aspect, an avatar reaction can include sending an electronic message (e.g., email, text message, etc.), or initiating a phone call to another entity via the client device (e.g., client device 106) employed to render the avatar.

For example, based on a determination by analysis component 212 that a user's heart rate is too high while performing a fitness routine, reaction component 214 can determine a response for manifestation by the avatar to direct the user to slow down. For example, the determined reaction to be manifested by the avatar can include a verbal spoken command to “slow down the pace” as well as a physical demonstration by the avatar of the exercise at the correct pace. In another example, in response to a determination by analysis component 212 that a user participating in an alcohol addiction recovery program has a blood alcohol level of X and/or is located at or near a bar, a response can be determined by reaction component 214 for manifestation by an avatar that includes verbal instruction criticizing the user for the bad behavior with corresponding facial expression and body language that showing an expression of disappointment. In addition, reaction component 212 can initiate calling of the user's alcohol anonymous (AA) sponsor and the avatar presented to the user can inform the user that a phone call is being initiated. In another example, in response to a determination that a user is likely to deviate from a diet program based on information indicating the user is stressed, the user is hungry, and the user is near another person that the user usually makes deviating food choices with on Monday afternoons (when the time is Monday 3 pm), reaction component 214 can determine a reaction for manifestation by an avatar that calls the user's attention to the scenario, and provides the user with breathing exercises to perform with the avatar as a guide to reduce the user's stress before the user's makes any poor decisions.

In another aspect, an avatar reaction can include provision of external data to the user at the client device (e.g., client device 106) employed to render the avatar. The external data can include but is not limited to: documents, articles, media, multimedia (e.g., video, animations, sounds, music, etc.), or hyperlinks to these data objects, that are accessible to avatar guidance platform (e.g., in memory 212, or at a remote source/system 238). For example, in association with an avatar reaction to facilitate encouraging a user to stick to an anti-smoking program, the avatar can provide the user with an article about the effects of smoking. In another example, to encourage a user to adhere to a fitness program, the avatar can provide a user with an image of herself when she used to weight 251bs more and an image of what she will look like at her goal weight.

In another example, in response to a determination by analysis component 212 that a user on a diet program is becoming stressed (e.g., based on cortisol levels) and has taken a walk to the vending machine (e.g., based on location and movement data), reaction component 214 can determine or infer a reaction for manifestation by an avatar that deters the user from making bad food choices. For example, reaction component 214 can determine a response that includes providing the user with motivation to stick to the diet program by playing a video of the user's past fitness dedication. The avatar can further speak to the user to encourage the user not to select the candy bar from the vending machine and rather go to the cafeteria to get a banana (e.g., a preferred snack option determined based on the user's food preferences and dietary restrictions).

In an aspect, reaction component 214 can determine or infer what action or inaction the user should do to correct an identified deviation and then determine or infer a proper reaction for manifestation by the avatar to direct the user to perform the action. For example, in response to a determination by analysis component 212 that a user has not mastered a particular golf swing (e.g., based on a determination that the user's physical motion indicates improper performance of the golf swing), reaction component 214 can determine that the user should switch to practicing a different swing and associated club. Thus, reaction component 214 can determine a response for manifestation by an avatar that includes verbal instruction telling the user to switch to the other club along with physical simulation of the new golf swing with the new club.

The particular response determined by reaction component 214 for manifestation by an avatar can be based on a plurality of factors and information. In an aspect, known deviations, deviation types and degrees of the deviations for a specific program, routine or activity can be defined and correlated to fixed avatar reactions. For example, for a fitness routine, if blood pressure is too high, the associated avatar response can include a slowing down movement. In addition, reaction component 214 analyze user profile information to tailor response to the particular user, (e.g., based on user preferences, user demographics, user health information, user behavioral history, etc.). Further, reaction component 214 can consider context information when determining a reaction for manifestation via an avatar to counterbalance a deviation (or adherence) to a program, routine or activity. For example, when reaction component 214 determines that a user should perform an action to reduce stress (e.g., based on a determination that the user's stress level is too high in association with a stress reduction program), reaction component 214 can determine the action based on the user's location, weather conditions, time of day, etc. For instance, when it is raining, the action can include performing yoga indoors, yet when it is not raining, the action can include taking a walk outside.

In an aspect, reaction component 214 can employ various defined response rules 224 that relate known deviations and degrees of the deviations for a specific program, routine or activity, with user profile information and context information to a plurality of possible pre-defined avatar reactions. For example, the response rules 224 can include a set of prefixed avatar responses that are mapped to potential determinations of deterrence and/or adherence to a particular activity or program. These prefixed responses are specific to the program, routine, or activity being monitored. In an aspect, prefixed responses are defined by an administrator that created the activity or program guidelines.

In other aspects, reaction component 214 can modify/adapt prefixed avatar reactions and/or develop new avatar reactions based on the specific program, routine or activity, the determined deviation (or adherence), user profile information (e.g., user preferences, historical user behavior, user health information, etc.), user context information, and known capabilities of the user, using various classification (explicitly and/or implicitly trained) schemes and/or systems (e.g., rule based classification schemes, support vector machines, neural networks, expert systems, Bayesian belief networks, fuzzy logic, data fusion engines, etc.).

Processing module 210 can include inference component 216 to provide for or aid in various inferences or determinations associated with aspects of avatar guidance platform 202. In an aspect, all or portions of avatar guidance platform 202 can be operatively coupled to inference component 216. Moreover, inference component 216 can be granted access to all or portions of media provider, remote sources/systems 238, client device 106 and other sources accessible via a network 112.

In an aspect, analysis component 212 can employ inference component 216 to infer whether, how and to what degree a user deviates from a specific program, routine or activity. In association with such inferences, inference component 216 can examine determined physical and physiological activity feature values and reference physical and physiological activity feature values, determined context parameters and reference context parameters, user profile information, and other context information. In addition, reaction component 212 can employ inference component 216 to infer a response for manifestation via an avatar that facilitates adherence to a program, routine or activity. Reaction component 214 can also employ inference component 216 to infer an action a user should perform to correct a deviation from a program, routine or activity as well as a reaction for manifestation via an avatar the directs and/or motivates the user to perform the action. In association with such inferences, inference component 216 can examine deviations that have been determined/inferred by analysis component 212, information relating such deviations to predetermined avatar responses, user profile information, and user context information.

In another aspect, analysis component 212 can employ inference component 216 to infer foreseeable violations/deviations to a program, routine, or activity which the user is following. For example, where a user on alcohol addiction recover program is headed into an area with several triggers for the user (e.g., bars at happy hour time) and the user has high cortisol levels, inference component 216 can infer that the user is likely to be inclined to fall off his program and stop at a bar. Based on inferences identifying foreseeable violation/deviations, reaction component 214 can employ inference component 216 to infer mechanisms to circumvent the occurrence of the foreseeable violations/deviations and infer reactions for manifestation via an avatar to facilitate relaying these mechanisms to the user. In furtherance to the subject example, reaction component 214 can infer that a call to the user's AA sponsor and warning message would help deter the user from violating his program. Thus, reaction component 214 can initiate a response for manifestation by the avatar that includes a call to the AA sponsor and a message delivered with a suitable tone of voice and facial expression warning the user not to violate his program.

In order to provide for or aid in the numerous inferences described herein, inference component 216 can examine the entirety or a subset of the data to which it is granted access and can provide for reasoning about or infer states of the system, environment, etc. from a set of observations as captured via events and/or data. An inference can be employed to identify a specific context or action, or can generate a probability distribution over states, for example. The inference can be probabilistic—that is, the computation of a probability distribution over states of interest based on a consideration of data and events. An inference can also refer to techniques employed for composing higher-level events from a set of events and/or data.

Such an inference can result in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines, etc.) can be employed in connection with performing automatic and/or inferred action in connection with the claimed subject matter.

A classifier can map an input attribute vector, x=(x1, x2, x3, x4, xn), to a confidence that the input belongs to a class, such as by f(x)=confidence(class). Such classification can employ a probabilistic and/or statistical-based analysis (e.g., factoring into the analysis utilities and costs) to prognose or infer an action that a user desires to be automatically performed. A support vector machine (SVM) is an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

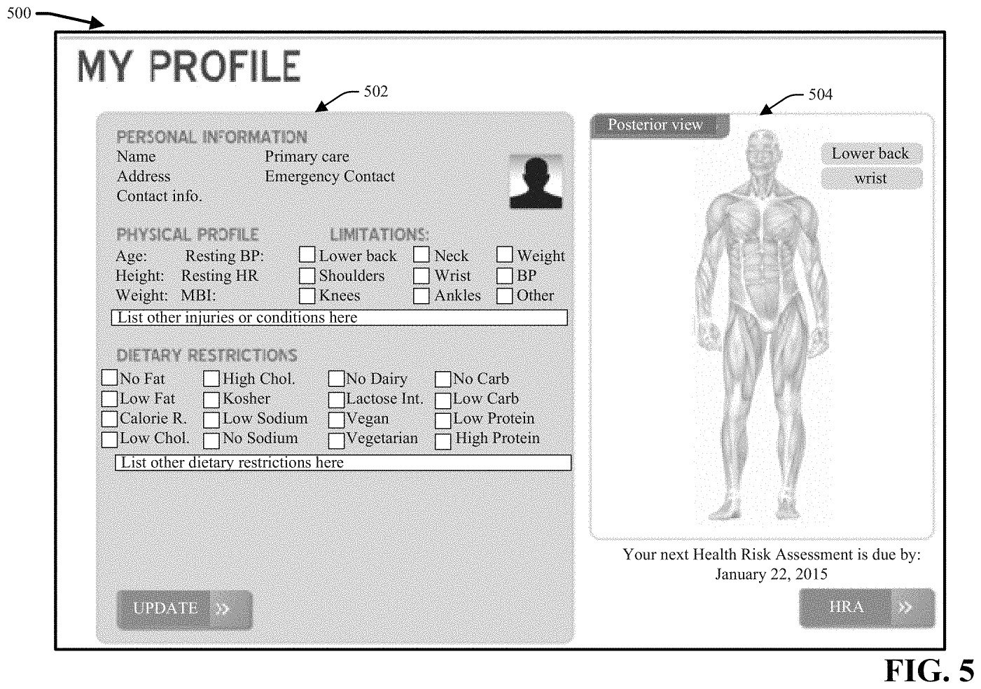

Avatar guidance platform 202 can further include interface component 206, avatar control component 208 and avatar generation component 226. Interface component 206 configures a graphical user interface(s) that facilitates user interaction with system 200. In particular, interface component is configured to generate a graphical user interface that includes an avatar that performs the various reactions discussed herein. The graphical user interface is presented to the user via rendering component 236 located at a client device (e.g., client device 106) employed by the user. In some aspects, the interface can provide mechanisms to select or create a program, routine or activity that a user would like to perform and have monitored via avatar guidance platform 202. The interface can further provide mechanisms for receiving input from a user related to a profile of the user and allowing the user to access and view his or her profile information. Some exemplary interfaces configured by interface component 206 in association with application of avatar guidance platform 202 to facilitate guiding a user though a fitness routine or activity are presented in FIGS. 5-7.

Avatar control component 208 is configured to interpret a response or reaction determined by reaction component 214 for manifestation by an avatar presented to a user and determine control commands to provide to avatar generation component 226 to effectuate the response by the avatar. Avatar generation component 226 is configured to generate an avatar that performs the response/reaction based on the control commands. In particular, avatar generation component 226 is configured apply the control commands to an avatar displayed to a user and generate an avatar performing the response/reaction. In essence, avatar generation component 226 is configured to generate an animation with an avatar moving, speaking, and/or appearing based on control commands received from avatar control component 208.

As previously described, in some aspects, the avatar guidance platform 202 can be located at the client device (e.g., client device 106) that includes rendering component 236. In another aspect, one or more components of avatar guidance platform 202 are located at a remote server (e.g., avatar server 116). For example, interface component 206, avatar control component 208, and avatar generation component 226 can be located at a remote server. According to this aspect, a user can access an interface, (e.g., using client device 106 for display at the client device) including the avatar performing various responses based on the user's input 234 via a network (e.g., at a website or other networked platform). The animation including the avatar can be provided to the client device for rendering as streaming video streamed thereto from the server. In another aspect, one or more components of avatar guidance platform 202 can be distributed between the server and the client. For example, a remote server can include the avatar control component 208 while the client device can include the avatar generation component 226. According to this example, avatar control component 208 can send the control commands to the client device via a network. Upon receipt of the control commands, the avatar generation component 226 at the client device can generate the avatar animation based on the control commands.

In an aspect, an avatar generated in association with providing guidance and instruction to a user with respect to performance of a program, routine, or activity can inform the user via visual and/or audible (e.g., speech) responses how to perform the selected activity or program prior to beginning the program/activity and/or over the course of the program/activity. For example, when the selected activity is a fitness routine, an avatar can be generated and presented to the that performs at least part of the fitness routine so that the user can copy or follow the avatar. In another example, the user can have knowledge of what an activity or program requires (e.g., a diet program, a daily routine, an addiction recovery program) and an avatar generated to guide adherence to the activity or program can simply respond to the user's adherence or non-adherence to the requirements of the activity or program.

As previously noted, avatar guidance platform 202 can be employed to guide a user in association with performance of a variety of different programs, routines and activities. In one exemplary embodiment, avatar guidance platform 202 is employed to facilitate guiding a user through a fitness routine or activity. Avatar guidance platform 202 can include fitness module to provide various features specific to this application. In another exemplary embodiment, avatar guidance platform 202 is employed to provide a user with a personal guide or assistant to facilitate adherence to a program or schedule. According to this embodiment, avatar guidance platform 202 can include program guide module 232 to provide various features specific to this application. Fitness module 230 and program guide module 232 are discussed in greater detail infra.

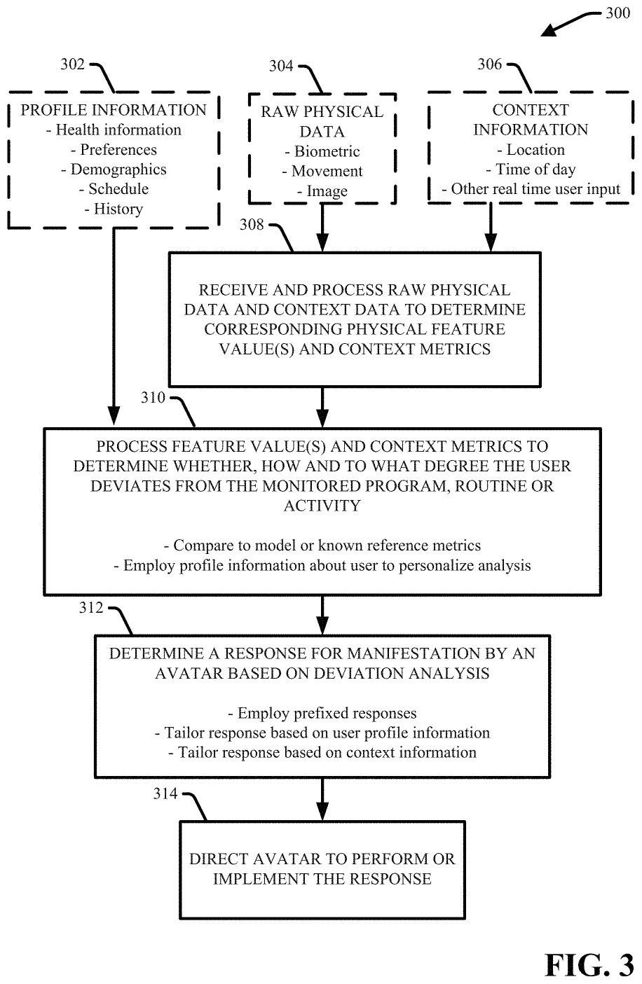

FIG. 3 provides a high-level flow diagram 300 of a process for guiding or assisting a user with adhering to a program, routine or activity using an avatar in accordance with various aspects and embodiments described herein. Repetitive description of like elements included in respective embodiments of systems and methods described herein is omitted for sake of brevity.

Process 300 involves receiving and processing various input data about a user's physical state and activity along with various other personal and contextual information to facilitate guiding and assisting the user with performance of a particular program, routine or activity via manifestation of responses via an avatar based on the input. In particular, at 308, raw physical and physiological activity data 304 and context information 306 for a user is received and processed to determine corresponding physical and physiological activity feature values and context metrics/parameters, respectively. The raw physical and physiological activity data 304 can include biometric data, movement data, and/or image data captured for the user via various sensor devices attached to the user (e.g., sensor device 104) and/or various auxiliary devices (e.g. intelligent fitness device(s) 118, visual capture device 110, sensor module 112, etc.) as the user performs a specific program, routine or activity. The context information 306 can include location information (including information about the location, such as weather, events, physical structures, etc.), time of day, and other real-time user input (e.g., input provided by the user regarding the user's thoughts, feelings, desires, etc.).

For example, based on received physical and physiological activity data 304, at 308 various information about a user's physiological state can be determined (e.g., heart rate, blood pressure, temperature, calories burned, blood alcohol level, cortisol level, glucose level, etc.). In addition, based on received motion/movement data and/or image data over a period of time, pattern analysis can be employed to determine what physical movements the user is performing (e.g., jumping, running, performing a specific yoga pose, performing a chest press, etc.) as well as characteristic about the movements (e.g., body position, speed, pressure, intensity, etc.).

At 310, the physical and physiological activity feature values and context metrics are processed to determine whether, how and to what degree the user deviates from the monitored program, routine or activity. This processing can involve evaluation of the physical and physiological activity feature values and context metrics in view of model or reference physical and physiological activity feature metrics and/or reference context metrics for the monitored program, routine or activity. For example, this processing can include comparison of determined physical and physiological activity feature values and/or context metrics to corresponding reference metrics for the monitored program, routine or activity to determine whether, how and to what degree the user's performance and behavior deviates there from. In an aspect, this processing can involve various classification schemes and that relate different combinations of physical and physiological activity feature values to one another based on the corresponding reference metrics to determine or infer whether, how and to what degree the user deviates from the monitored program, routine or activity.

In addition to reference metrics, processing at step 310 can also involve analysis of received physical and physiological activity feature values and/or context information in view of requirements for these values in association with performance of the monitored program, routine or activity, and based on user profile information 302. This profile information can include but is not limited to: user health information, user preferences, user demographics, user schedule, and user performance history in association with the monitored program, routine or activity, and/or other monitored programs, routines or activities for the user. According to this aspect, the physical and physiological activity and contextual requirements for a program, routine, or activity can be tailored to account for a user's physical capabilities, preferences and goals, demographics, schedule, or history. Accordingly, where two different users perform the same routine and exhibit the same physical and physiological activity feature values, one user can be determined to deviate the program while another could be determined to adhere to the program.

At 312, based on a determination of whether, how, and to what degree a user deviates from a monitored program, routine or activity, a response is determined for manifestation by an avatar. The response is specific to the deviation (or determination of no deviation), the degree of the deviation, the program, routine or activity being monitored, the user, and potentially the context of the user. In aspect, an avatar response/reaction is determined or inferred based in part on various prefixed responses for the monitored program, routine, or activity and the particular deviation, user profile information 302 and context information 306. The response is specifically designed to provide the user with guidance in association with adhering to the monitored program, routine or activity. For example, where a user deviates from a monitored program, the response can include verbal and/or visual instruction by the avatar to command and/or motivate the user to perform an action to correct or accommodate the deviation. In another example, where it is determined that the user is properly adhering to a monitored program or activity, a response could include a showing of praise and encouragement by the avatar. In another example, where it is determined at step 310 that the user is likely to deviate from a program in the foreseeable future (e.g., by likely eating bad food based on a determination the user is located at a fast-food restaurant), the response can include a mechanism to prevent the user from deviating. For example, such a mechanism could include provision of motivational coaching, alternative options, or imagery and/or external source data by the avatar that is selected to persuade the user to stick to the program.

After a response is determined at step 312, an avatar presented to the user is directed to perform or implement the response. For example, avatar generation component 226 can generate and avatar that performs the response based on control commands provided by avatar control component 208. These control commands are specifically designed by avatar control component 208 to cause the avatar to perform the determined response. For example, when the avatar response includes speaking a phrase while performing a specific movement and smiling, the control commands can direct avatar generation component 226 to generate an avatar that speaks the phrase while performing the specific movement and smiling.

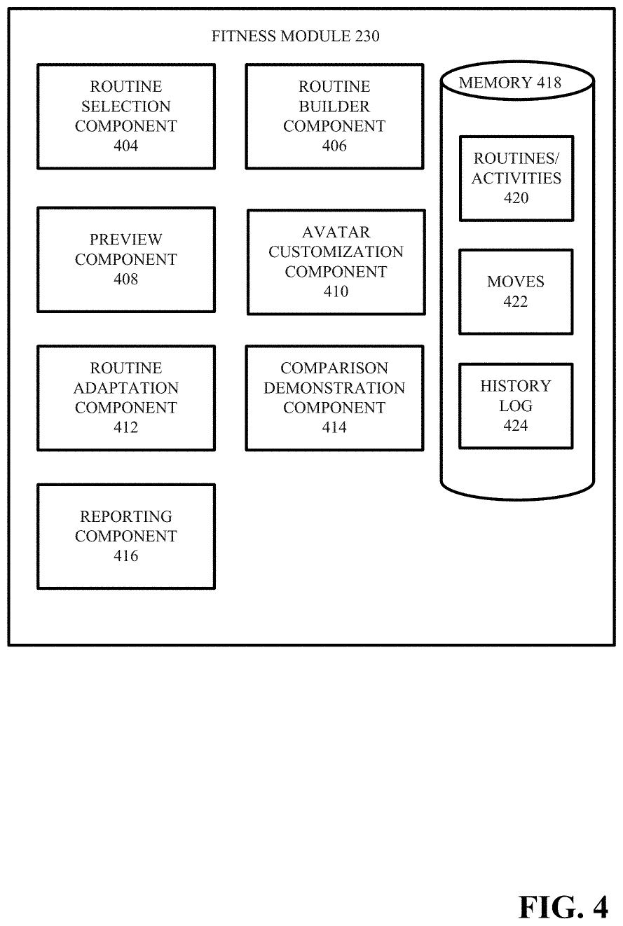

FIG. 4 provides an example embodiment of fitness module 230 in accordance with various aspects described herein. Repetitive description of like elements included in respective embodiments of systems and methods described herein is omitted for sake of brevity.

Fitness module 230 is specifically configured to facilitate guiding or assisting a user with adhering to fitness routine or activity using an avatar in association with various aspects of avatar guidance platform 202. For fitness applications such as physical therapy, personal training, aerobics, performing a sports activity (e.g., gymnastics, golf, etc.), etc., the avatar plays the role of a virtual physical therapist, personal trainer or teacher. During the routine or activity, the user's physical inputs (e.g., physiological, motion, and/or image input) will automatically control the behavior of the avatar. The behavior of the avatar is designed to guide or assist the user with the fitness routine or activity. For example, the avatar's behavior can resemble that which a real trainer would exhibit to provide instruction, demonstration, motivation, praise, coaching, etc.

In particular, as a user performs a specific fitness routine or activity, the user's physiological input is received by avatar guidance platform 202. The user's physical and physiological activity input is further processed by processing module 210 as it is received (in real-time or near-real time) to evaluate the user's adherence to the specific fitness routine or activity (e.g., by analysis component 212), in accordance with aspects described herein. Based on this evaluation, responses/reactions are determined for manifestation by an avatar (e.g., by avatar reaction component 214), in accordance with aspects described herein. Avatar control component 208 and avatar generation component 226 then cause an avatar presented to the user to perform the response/reactions.

For example, in accordance with various aspects of avatar guidance system 200 and fitness module 230, a user can begin performance of a selected or assigned fitness routine. An avatar is presented to the user (via a graphical user interface or as a hologram) to help guide the user through the routine and serve as the user's coach or trainer. Depending on the selected fitness activity or routine, the avatar can be configured to perform some or all of the fitness routine with the user so that the user can mimic the movements of the avatar. In another aspect, the avatar can be configured to direct the user to perform various actions in accordance with the routine and provide demonstration of physical movements of the routine movements when necessary.

As the user performs the routine, avatar guidance platform 202 receives physical and physiological activity input data for the user in accordance with aspects described herein. For example, when the user is wearing a heart rate monitor, information, identifying the user's heart rate is obtained. This information along with other potential physical and physiological activity data related to the user's physiological state, movement and appearance is evaluated as it is received to determine whether, how, and to what degree the user's performance deviates from the physical and physiological activity requirements of the fitness routine in view of personal characteristics of the user, such as physical restrictions/capabilities of the user. In an aspect, the user's physical and physiological activity data is compared or analyzed based on reference information for the routine defining requirements for the physical and physiological activity data in association with performance of the physical routine (e.g., physiological metrics associated with respective movements and/or times throughout the routine, actions the user should perform to accomplish the routine, when to perform them and how to perform them with respect to range or motion, body position, intensity, etc.). Analysis of these reference metrics is further adapted based on user profile information (e.g., user physical restrictions, user physical capabilities, user demographics, etc.).

Continuing with the subject example, during performance of a certain movement of the fitness, the user's heart rate is determined to be 100 beats per minute where the routine requires a person having the height, weight and age of the user to have a heart rate of 125 beats per minute. Thus, analysis component 212 determines that the user deviates from the routine based on having a heart rate below the optimal heart rate. Based on this deviation, reaction component 214 determines a reaction for manifestation by the avatar presented to the user to encourage the use to increase his heart rate to 125 beats per minute. In an aspect, reaction component 214 can base this determination on the specific physical routine, the type of deviation and degree of the deviation, prefixed responses for the routine and deviation, user profile information and potentially context information. Avatar control component 208 and avatar generation component 226 can then cause the avatar to perform the reaction. Continuing with the subject example, the avatar may visually increase the pace of the motion, provide verbal instruction to increase speed, or a provide a visual or audible indicator that increases the rhythm of the motion to encourage the user to match the pace. For instance, when performing alternating lateral lunges (mimicking the motion of speed skating as they are also known) when moving side to side, one's heart rate should increase. If the target heart rate is not met, the speed at which an avatar demonstrating the exercise is lunging side to side will increase until the targeted heat rate is met. Conversely, if the user's heart rate exceeds the target zone, the avatar will slow down until the user's heart rate also drops. In another example, the avatar can change aspects (e.g., the rhythm, the song, the volume, etc.) of music playing to motivate the user to increase his pace.

By customizing and automatically responding to changes in the user's biometric data, the avatar guidance system 200 improves user safety during physical exercise. In particular, the avatar guidance system 200 is configured to respond to user input automatically and in real-time, thereby providing indication of a potentially dangerous activity in advance of an unsafe result. For instance, if the system recognizes certain metrics as leading to bodily harm, the system can instruct the avatar to stop the workout session and contact a health professional or automatically send a notification (e.g., a call, electronic message, etc.) to the health professional. If necessary, the system can additionally or alternatively be configured to contact emergency services, thus saving precious time in the event of a severe or potentiality life threatening injury.

To provide for various aspects of avatar guidance system 200 when applied to facilitate a fitness routine or activity, fitness module 230 can include routine selection component 404, routine builder component 406, preview component 408, avatar customization component 410, routine adaptation component 412, comparison demonstration component 414 and reporting component 416. Fitness module 230 can also include various data objects stored in memory 418 (which can be inclusive of memory 218) that are specific to aspects of fitness module. These data objects can include information defining various fitness routines/activities 420 and activities capable of being monitored by fitness module 230, information defining various fitness movements or moves 422 capable of being performed by an avatar in association with a fitness routine or activity, and historical information (i.e., history log 424) for respective users logging their performance of fitness routines or activities using fitness module 230.

Routine selection component 404 is configured to allow a user of fitness module 230 to select a preconfigured fitness routine or activity to perform from a database of routines/activities 420. Each of these routines or activities have a defined set of physical moves and requirements for the user to perform. For example, the routine or activity can include an aerobics routine, a physical therapy session, a strength training exercise, a yoga routine, a dance routine, a golf exercise, a running workout, a biking workout, kickboxing workout, etc. The routine or activity can be designed for performance in a fixed space indoors or outdoors, over changing indoor or outdoor terrain, and/or using various equipment or apparatuses.

In another aspect, rather the selecting a stock fitness routine or activity to perform, routine builder component 406 can facilitate designing a custom routine for the user to perform. In an aspect, routine builder component 406 can allow the user (or a supervisor of the user), to build a custom fitness routine or activity by selecting various fitness moves to include in the routine or activity and selecting physical and physiological activity parameters associated with performance of the fitness moves. For example, using a database of a plurality of known physical movements capable of being performed by a human in association with a wide variety of fitness activities (e.g., moves database 422), the user can pick and choose various moves and/or combinations of moves to include in a fitness routine. For instance, when designing an aerobics routine, the user can select different aerobics moves can combinations of moves from a database of possible moves. In association with selecting moves, the user can also select characteristics associated with performance of the moves, such as intensity, duration, frequency, range of motion, speed, etc. For example, when designing a strength training routine, the user can select what weight-lifting movements to perform, weight to employ for each movement, number of repetitions, number of sets, range of motion, etc.

Still in yet another aspect, routine builder component 406 can be configured to design a custom fitness routine or activity for a user based on the user's needs, desires, physical abilities and context. In particular, avatar customization component 410 can design a customized fitness routine or activity for a user to perform based on user profile information including but not limited to: information describing the user's demographics, physical profile (e.g., height, weight, physical abilities, physical injuries or restrictions, etc.), preferences (e.g., what types of activities the user prefers or dislikes), goals (e.g., fitness activity performance goals, weight loss/gain goals, muscle building goals, etc.), and monitored physical performance history, (e.g., history of fitness routines/activities the user has completed and summary of the user's performance, as provided in history log 424). For example, based on user profile information, routine builder component 406 can design a customized routine for the user to perform that includes moves the user is capable of safely performing, moves the user prefers, moves that will challenge the user and facilitate achieving the user's goals, moves that the user did not perform yesterday, etc.

In addition, avatar customization component 410 can analyze context information when designing or selecting a customized routine or activity for a user to perform. For example, avatar customization component 406 can consider the user's location, the time of day, the amount of time available to the user, the current weather associated with the location, and fitness equipment/apparatuses available to the user. In an aspect, in association with designing a customized routine for a user, routine builder component 406 can receive user input providing some desired characteristics for the routine. For example, the user can provide information regarding a type of activity the user would like to perform that day, (e.g., cardio, weight training, yoga, kickboxing, running/biking outside, etc.), one or more muscle groups the user would like to work on, the desired duration of the activity.

In an aspect, an avatar generated and presented to a user in association with performance of a fitness routine or activity can perform the movements of the fitness routine or activity. While designing a fitness routine or activity and/or prior to performing a fitness routine or activity, the user may desire to see a demonstration of one or more of the physical moves required by the activity. Preview component 408 is configured to generate an avatar that demonstrates one or more moves selected for inclusion in a fitness routine or activity prior to beginning performance of the routine or activity. For example, a user can select a stock routine or activity and/or select a custom routine that has been designed for the user (e.g., automatically by routine builder component 406) and then select preview component 408 to be presented with an avatar that demonstrates one or more of the moves included in the selected routine or activity. Based on the preview, the user can decide whether or not to proceed with performance of the selected routine or activity.

In another aspect, while designing a custom fitness routine or activity, the user can view an avatar demonstrating a chosen move or combination of moves based on the requirements selected for the move or combination of the moves (e.g., speed, intensity, range of motion, etc.). For example, the user can select a series of yoga poses to include in a yoga routine and the select preview component 408 to view the series of yoga poses being performed by an avatar. If the user is unsatisfied with the manner in which the poses flow based on the preview, the user can change aspects of the selected pose series. In an aspect, when viewing the previewed demonstration of a fitness move or moves, preview component 408 can allow the user to change the perspective of view of the avatar. For example, the user can rotate a virtual camera pointed at the avatar to various viewpoints (e.g., 360° with respect one or more axis) of the avatar so that the user can view the avatar's body position during performance of the fitness move from various points of view.

Avatar customization component 410 allows a user to customize the avatar presented to the user to facilitate a fitness routine or activity. In an aspect, avatar customization component 410 can allow the user to manipulate variables to create an avatar that reflects the needs and tastes of the user. In particular, using avatar customization component 410, a user can select the avatar's appearance, demographics, voice and personality. For example, the age, gender, language or accent, dress, or other visual and/or audio characteristic of the avatar may be selected to motivate and/or comfort the user. In another aspect, avatar customization component 410 can provide predetermined character personas for the user to select and apply to his or her avatar. For example, the avatar can be selected from a familiar character set that includes known cartoon characters or people (e.g., famous actors, musicians, politicians, athletes, etc.) where such characters creators or persons have authorized usage of their persona. For example, a cartoon avatar may be suitable to lead a child user through an exercise regime, or a popular athlete or fitness trainer may motivate an adult user to adhere to a fitness program.

In another aspect, avatar customization component 410 can automatically design an avatar to facilitate a user with a fitness routine or activity. According to this aspect, avatar customization component 410 can select the avatar's appearance, demographics, voice and personality based on one or more of: the fitness routine or activity selected for performance, profile information for the user regarding the user's preferences, the user's demographics, and the user's performance history with respect to monitored fitness routines or activities. For example, based on analysis of the user's preferences and demographics, avatar customization component 410 can determine or infer what type of avatar in terms of appearance and personality would best facilitate/motivate the user in association with performance of a selected fitness activity or routine (e.g., based on data relating avatar appearance and character traits to various aspects of user profile information).

In an aspect, an avatar configured to facilitate performance of a fitness routine or activity be configured to perform the fitness routine or activity so that the user can mimic the avatar. For example, a selected fitness routine or activity has known movements and requirements that can automatically control the movements of the avatar. In another aspect, avatar customization component 410 can allow a user to select the manner and degree to which the avatar performs the moves of a fitness routine or activity. For example, using avatar customization component 410, the user can select the degree of physical demonstration and instruction the user would like his or her avatar to provide. According to this example, the user could select full performance mode wherein the avatar is configured to perform the fitness routine or activity in full, or partial performance mode wherein the avatar is configured to perform parts of the routine or activity (e.g., provide a short demonstration of each new movement of a fitness routine or activity, provide demonstration of a move during a fitness routine or activity only upon request of the user during the course of the routine or activity, etc.). In another example, the user could select instruct mode wherein the avatar is configured to provide only verbal instruction regarding performance of a fitness routine or activity with the exception of a physical movement demonstration reaction determined by reaction component 214. In another example, avatar customization component 410 can allow the user to select correction mode wherein the avatar is configured to only demonstrate a move when the user performs it improperly.

Routine adaptation component 412 is configured to dynamically adapt aspects of a fitness routine or activity being performed by a user based on received physical and physiological activity data for the user, user profile information and potentially user context information. For instance, in some aspects an avatar's reaction to user performance deviating from a fitness routine or activity includes a visual or verbal cue to help or motivate the user correct the deviation. For example, when a user is moving too fast or too slow, the avatar's reaction is designed to cause the user to slow down or speed up, respectively. In other aspects, an avatar's reaction to user performance adhering to the fitness routine or activity (e.g., the user is performing the routine correctly with respect to physical and physiological activity requirements of the routine) is designed to simply provide praise to the user and encourage the user to keep up the good work.

However, in various additional aspects, an avatar's response to a user's performance deviating from or adhering to a fitness routine or activity can involve adaptation of requirements of the routine or activity, changing of the fitness routine or activity, and/or stopping of the fitness routine or activity. For example, in some situations where a user's physical and physiological activity performance is below standard, rather than pushing the user to improve his or her performance, it may be more appropriate to have the user to take a break and rest, stop performance altogether (e.g., where physical and physiological activity input is indicating the user is at risk for physical injury), have the user perform an easier version of the fitness routine, or have the user perform an different routine or activity. In another example, in situations where a user's physical and physiological activity performance is above standard and/or consistently on point, rather than merely praising the user for the good performance and encouraging the user to keep it up, it may be more appropriate to increase the intensity or difficulty level of the fitness activity or have the user advance to a new fitness activity that focuses on a new muscle group or skill set.

According to this aspect, fitness module 230 can include routine adaptation component 412. Routine adaptation component 412 is configured to dynamically determine whether and how to adapt or change the physical and physiological activity requirements of a fitness routine or activity during performance of the fitness routine or activity by a user, and/or whether and how to change the fitness routine or activity being performed by the user during performance of the fitness routine or activity be the user. In response to a determination to adapt a fitness routine or activity during performance of the fitness routine or activity by the user, routine adaptation component 412 can direct processing module to evaluate the user's performance based on the adaptation (e.g., new physiological and/or motion or pressure reference metrics). In addition, routine adaptation component 412 and/or reaction component 214 can determine a reaction for manifestation via the avatar to inform the user regarding the adaptation and facilitate performance of the routine by the user with the adaptation. This determined reaction can be automatically effectuated by avatar control component 208 and avatar generation component 226 during performance of the fitness routine or activity by the user.

In an aspect, routine adaptation component 412 is configured to make determinations regarding whether and how to adapt a fitness routine or activity based on analysis of a degree, consistency and frequency of a deviation or non-deviation during performance of the routine (e.g., with respect to degree, consistency and frequency thresholds for the deviation in association with performance of the routine), user profile information and/or context information. For example, routine adaptation component 412 can be configured to adjust the requirements of a fitness routine or activity based on consistent performance of the fitness routine or activity below standard performance, above standard performance, or at standard performance, wherein consistency is evaluated based on reference metric thresholds for frequency and duration of the deviation or non-deviation and/or degree of the deviation. For example, where a user consistently performs below the required number of reps on a weight machine, routine adaptation component 412 can determine that the amount of reps and/or weight should be lowered. In another example, where a user continually throws farther than a required distance, routine adaptation component 412 can determine that the required distance should be increased. In yet another example, where a user consistently performs a fitness move at a required intensity yet does not have an increase in heart rate, routine adaptation component 412 can determine that the required intensity of the fitness move should be increased.

Routine adaptation component 412 can also base a determination regarding adjustment of requirements of a fitness routine or activity based on user profile information, including but not limited to: historical performance of the user in association with performance of monitored fitness routines or activities, goals of the user, current physical state of the user, preferences of the user, and/or physical capabilities and limitations of the user. For example, where it is determined that a user is consistently completing reps on a weight machine at a required weight and the user has previously been able to lift more weight (in a past workout), routine adaptation component 412 can adapt the requirements of the routine to increase the number of reps and/or weight. In another example, where a user is continually nailing performance of a certain yoga pose during performance of a selected yoga routine, routine adaptation component 412 can change the selected yoga routine to include a more advanced pose. When selecting the more advanced pose, routine adaptation component 412 can consider poses that are safe for the user to perform in light of a known back injury and certain yoga poses that the user prefers or does not prefer. In yet another example, when a user is becoming extremely fatigued during a cardio aerobics routine based on consistent performance below the physical and physiological activity requirements of the routine, routine adaptation component 412 can adjust the intensity requirements of the aerobics routine by including easier moves the user prefers or change a muscle group focused on (e.g., from legs to abdominals) based on knowledge that the user has not worked the new muscle group in over a week.

Routine adaptation component 412 can also consider contextual information when determining whether and how to adjust requirements of a fitness routine or activity during performance of the fitness routine or activity. For example, when a user is running outside and the temperature increases by N degrees, routine adaptation component 412 can adjust the required speed or duration of the run to accommodate for the intensity increase associated with the rise in temperature. In another example, during performance of a fitness activity, the user may provide feedback stating, “this is too hard,” or “this is too easy.” Based on such feedback and information regarding whether and to what degree the user is deviating from the requirements of the fitness activity, routine adaptation component 412 can determine whether and how to adjust the requirements of the fitness activity.

In response to a determination by routine adaptation component 412 to change the requirements of a fitness routine or activity (e.g., change the speed, intensity, exercise move, range of motion, difficulty level, muscle group worked, etc.) and/or change the fitness routine or activity all together (e.g., stop performance or select a new routine or activity for performance by the user), routine adaptation component 412 can adapt the requirements of the routine accordingly. As a result, adherence to the fitness routine or activity will be evaluated (e.g., by analysis component 212) based on the adapted requirements. In addition, the avatar presented to user can provide a visual and/or audible response that informs the user regarding the adaptation. In particular, based on a change to a fitness routine or activity determined by avatar adaptation component 412, routine adaptation component 412 and/or reaction component 214 can determine a reaction/response for manifestation by the avatar that informs the user regarding the change and/or facilitates performance of the routine with the change. This reaction can involve a verbal cue informing the user about the change and/or a physical demonstration by the avatar of the routine or activity with the change.

For example, the avatar can demonstrate a new move or demonstrate a move at a different intensity while informing the user how to perform the new move or the move at the different intensity level. In some aspects, when an avatar is configured to perform the fitness routine for the user to mimic (e.g., an aerobics routine), the avatar can perform the fitness routine as adapted by routine adaptation component 412. For example, while performing a selected fitness dance routine, the avatar can dynamically change the moves, intensity of the moves, music associated with the routine, etc. over the duration of the routine based on determinations by routine adaptation component 412.

In an aspect, fitness module 230 can include reporting component 416 to provide real-time feedback regarding a user's performance of a fitness routine or activity to a remote entity, such as the user's human physical trainer or therapist at a remote location. For example, reporting component 416 can communicate any information received and/or processed by avatar guidance system to a remote entity via network as it is received and/or determined (e.g., in real-time). This information can include input 234 (e.g., raw physical and physiological activity data, user profile information, context information) received by avatar guidance platform, determinations regarding adherence or deviation from a fitness routine or activity performed by a user and determined/manifested avatar responses.

The remote entity can also communicate with avatar guidance platform 202 during performance of the fitness routine or activity by the user. Therefore, in an aspect, rather than having routine adaptation component 412 automatically determine and effectuate changes to a fitness routine or activity as the user performs it, a remote entity can determine such changes based on received information. The remote entity can further communicate the changes to routine adaptation component 412 and routine adaptation component 412 can effectuate the changes determined/requested by the remote entity. In particular, based on the changes to a fitness routine determined and communicated to fitness module 230 by the remote entity, adherence to the fitness routine or activity will be evaluated (e.g., by analysis component 212) based on the changes. In addition, the avatar presented to user can provide a visual and/or audible response that informs the user regarding the adaptation. In particular, based on a change to a fitness routine or activity determined by the remote entity, routine adaptation component 412 and/or reaction component 214 can determine a reaction/response for manifestation by the avatar that informs the user regarding the change and/or facilitates performance of the routine with the change.

Comparison demonstration component 414 is configured to generate a comparison visualization between an avatar performing a correct demonstration of a fitness move and a representation of the user (e.g., in an avatar form or a video replay of the user) as the user performed the move incorrectly. For example, if the user repeatedly performs a move incorrectly, avatar comparison component 414 can generate a second avatar configured to perform the move in the manner in which the user performed it incorrectly (e.g., based on motion data and/or image data collected for the user). In another aspect, the avatar comparison component 414 can collect video footage of the user as the user performed the move incorrectly. Comparison demonstration component 414 can the generate a side-by-side comparison visualization of an avatar performing the move correctly and another avatar representing the user (e.g., or the video footage of the user) performing the move incorrectly. Thus, a side-by-side comparison of the user's own actions and the virtual personal trainer model technique can be displayed.

FIG. 5 presents an example user interface 500 that facilitates receiving user profile information in association with employment of avatar guidance system 200 for physical fitness purposes in accordance with various aspects and embodiments described herein. Repetitive description of like elements included in respective embodiments of systems, methods and interfaces described herein is omitted for sake of brevity.

In an aspect, a user can establish a profile with avatar guidance system 200 that includes a variety of personal information related to the user and the user's usage of avatar guidance system 200. For example, when employing avatar guidance system 200 for fitness and health related purposes, a user can be present with interface 500 to facilitate establishing a user profile and entering various personal information related to the user's health and fitness profile. For instance, interface 500 include can information section 502 that facilitates receiving user input regarding the user's physical profile, the user's physical limitations, and the user's dietary restrictions. Interface 500 can also include section 504 that presents an interactive pictorial representation of a human with the various muscle groups displayed. In an aspect, using this section the user can select body parts and/or muscle groups to indicate where the user has injuries and/or physical limitations.

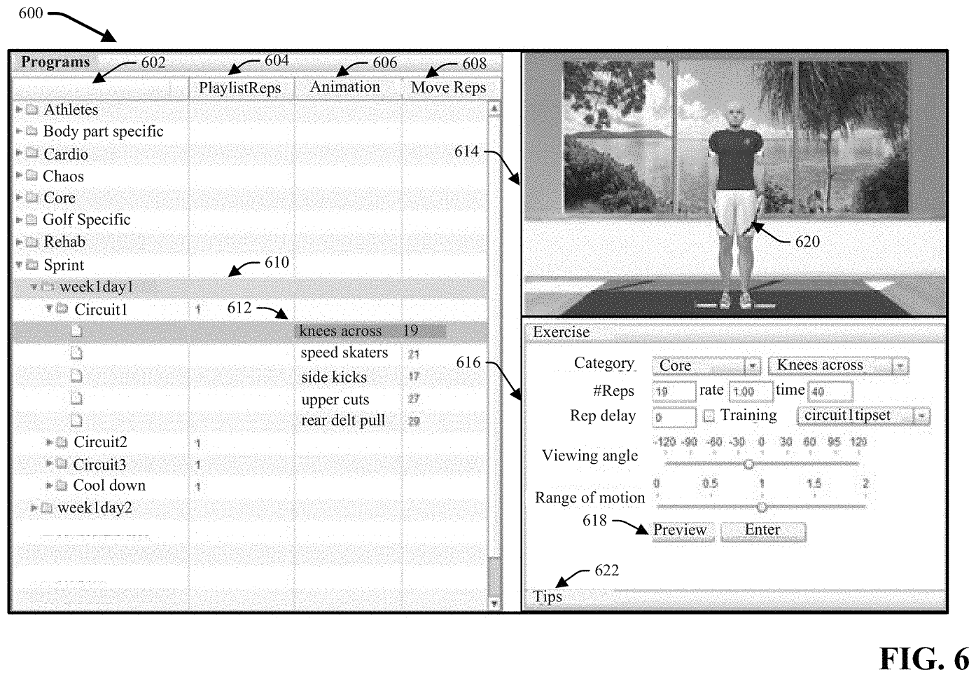

FIG. 6 presents an example user interface 600 that facilitates designing a custom fitness routine in association with employment of avatar guidance system 200 for physical fitness purposes in accordance with various aspects and embodiments described herein. Repetitive description of like elements included in respective embodiments of systems, methods and interfaces described herein is omitted for sake of brevity.

In various aspects, a user or an authorized supervisor of the user (e.g., the user's fitness trainer, physical therapist, doctor, parent, etc.), can design a custom workout to perform and have guided by an avatar presented to the user. For example, the user can design an exercise routine with various selected moves and characteristics and requirements for the moves, such as number of repetitions, time between repetitions, duration, frequency, range of motion, intensity, etc. Accordingly, the user can select/manipulate various variables to create a workout program specifically designed to match a user's needs, preferences, physical limitations, and goals.

Interface 600 provides an example tool for designing a fitness routine in accordance with aspects described herein. The left side of the interface includes column 602 which includes a plurality of different menu categories to select in association with designing a custom workout. In an aspect, column 602 provides the capability to choose a type of workout desired from a plurality of different menu options that correspond to categories of workout types, such as body part specific, cardio, chaos, core, golf specific, rehab, etc. For example, the ‘sprint’ workout category is selected in example interface 600. The user can further customize various aspects of a selected workout using various potential sub-category options available via a drop-down menu from a selected menu category of column 602. These sub-categories can vary depending on the workout category selected. For example, these sub-categories can correspond to different parts of a workout (e.g., warm up, cool down, high intensity portion, cardio portion, etc.), different machines/tools or weights used in association with the workout, different rounds or circuits of a workout, etc. In another example, these sub-categories can correspond information regarding when the workout is to be performed in association with a workout program. In other aspect, the user can define sub-categories of a workout type menu category.

As exemplified in interface 600 the user has selected the ‘sprint’ workout for week 1 day, 1 and week 1, day 2 of a workout program. The sub-category week 1 day, 1 is currently selected (as indicated by highlighted column 610). This sub-category includes additional drop-down sub-categories corresponding to different parts of the workout, including circuit 1, circuit 2, circuit 3 and cool down. The user can select each of the part of the workout and define exercise moves (and characteristics of the exercise moves) to include therein. For example, with respect to circuit 1, the user has selected the following exercise moves: knees across, speed skaters, side kicks, upper cuts and rear delt pult. In column 604, the user can select the number of repetitions to complete for the respective parts of the workout. For example, each of circuit 1, circuit 2, circuit 3 and cool down are selected for repeating once (as indicated by the number 1 next to each part of the workout under column 604.

Column 606 corresponds to animation. In particular, a description of the selected exercise move appears in column 606 to indicate what the move is at it will be demonstrated by an avatar presented to the user. Additional information describing how the animated avatar will appear when demonstrating the move in accordance with the defined characteristics of the move (e.g., speed, range of motion, etc.) can also be included in column 606. Column 608 corresponds to the number of exercise moves reps selected for completion. For example, the user has selected to perform 19 repetitions of ‘knees across.’

In an aspect, section 616 of interface 600 facilitates defining various characteristics of a selected exercise move and section 614 provides a video animation demonstration of an avatar 620 performing the selected exercise move in accordance with the selected characteristics. For example, on the left side of the interface, the user has move ‘knees across’ is currently selected (as indicated by highlighted row 612). Using section 616, the user can define the various requirements for the exercise ‘knees across,’ such as number of repetitions, rate of the repetitions, time to complete the repetition, amount of time delay between repetitions, whether the exercise is a training exercise, range of motion, speed of the exercise, etc. It should be appreciated that a variety of different feature requirement selections can be provided in section 616 (e.g., heart rate, blood pressure, speed, etc.) which can also vary depending on the selected exercise.

After a user has selected specific requirements for an exercise in section 616, the user can select the preview button 618 to see a preview demonstration of the avatar 620 displayed in section/window 614 performing the exercise in accordance with the specified requirements. In an aspect, as the user previews the avatar 620 performing the exercise, the user can change the viewing angle at which the avatar is displayed (e.g., of a virtual camera pointed at the avatar 620) to see how the movements are demonstrated from different views of the avatar. For example, depending on the demonstrated exercise, a side or front view may provide a more helpful perspective of the avatar model. In addition, section 616 includes a tips category 622, wherein the user can provide personalized tips, instruction, or messages to be provided to the user (e.g., in an audible format, spoken by the avatar, etc.) when the user performs the exercise. For example, the user can provide a description of what the move is, how to perform the move correctly, what not to do when performing the move, a motivational quote, etc.). These tips can be configured for rendering to the user (e.g., in an audible format, spoken by the avatar, etc.) when the user performs the specific exercise move during the workout.

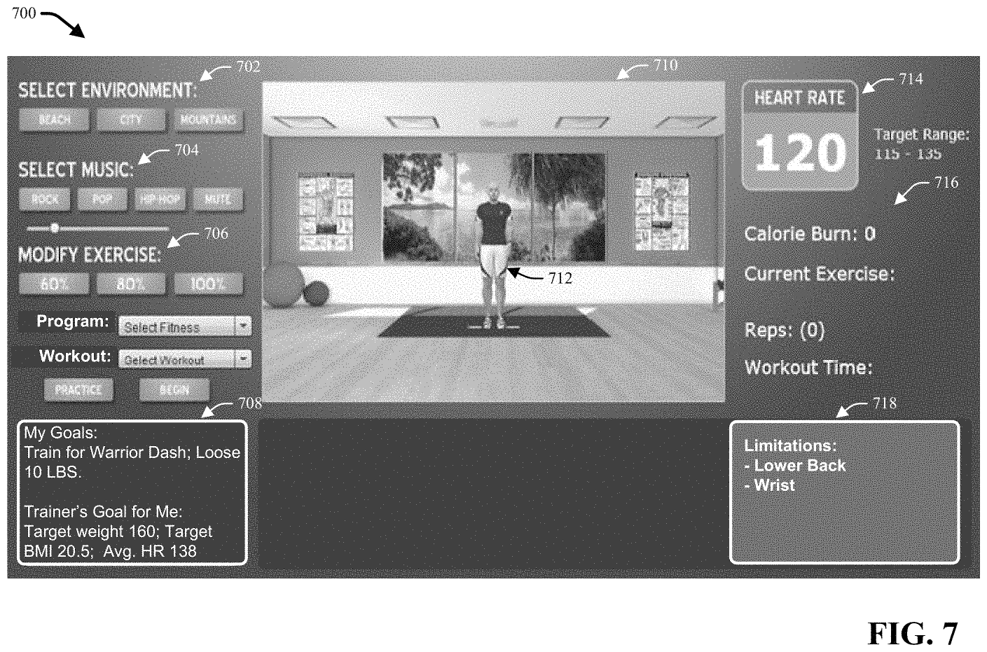

FIG. 7 presents an example user interface 700 that presents a user with an avatar to facilitate guiding the user through a selected fitness routine in accordance with various aspects and embodiments described herein. Repetitive description of like elements included in respective embodiments of systems, methods and interfaces described herein is omitted for sake of brevity.

In an aspect, interface component 206 can generate interface 700 for presentation to a user (e.g., via rendering component 236) during performance of a selected fitness routine or activity. Interface 700 includes an avatar 712 displayed within a workout space 710. The appearance of the avatar and/or the workout space are customizable (e.g., via avatar customization component 410). In an aspect, as described above, the avatar 712 is configured to function as the user's personal trainer and provide instruction to the user regarding performance of the selected fitness routine. In particular, the avatar 712 is configured to respond in real-time to physical and physiological activity input received for the user as the user performs the fitness routine in accordance with aspects described herein. For example, as the user performs a fitness routine, the avatar 712 can provide various real-time reaction to the user's performance based on received and analyzed physical and physiological activity data for the user (e.g., in accordance with aspects described herein). For instance, the avatar can call out commands, tell the user how to correct certain physical deviations from the routine (e.g., based on physiological data and/or movement data for the user), provide physical demonstration of moves, motivate the user with facial and body movement expression, etc. As described above, these reactions can be specifically tailored to the user's personal tastes, goals and abilities (e.g., based on user profile information) and/or the user's current context (based on received context information). In some aspects, the avatar 712 is configured to perform the fitness routine for the user to follow. In other aspects, the avatar can perform parts of the fitness routine during performance of the fitness routine by the user when demonstration is necessary (e.g., to correct improper technique by the user).

Interface 700 can also include various tools and menu options to facilitate customizing aspects of the workout routine and/or the appearance of the interface. For example, interface 700 can include a ‘select environment section’ 702 wherein a user can select a background environment (e.g., a beach, a city, mountains), for display in the workout space 710. Interface 700 can also include section a ‘select music section’ 704 which allows the user to select a type of music for playing during a workout. In an aspect, this section can be linked to another music database (e.g., the user's playlist or an external media streaming system) to allow for selection of a wide variety of music option. Section 706 includes a ‘modify exercise section’ wherein a user can modify various aspects of an exercise prior to or during performance. For example, the user can select the intensity of the workout (e.g., 60%, 80%, 100%, etc.). Modify exercise section 706 can also provide for selection of a fitness program and or workout to perform and/or modification of a selected workout program or routine. In order to begin a selected workout, the user can select the begin button. In an aspect, prior to beginning the workout, the user can select the practice button which takes the user through various simulations of the respective exercises in the routine as demonstrated by the avatar 712.

Interface 700 can also include various visual feedback areas (e.g., 714 and 716) that provide the user with information regarding the user's performance during the workout. For example, interface 700 can include a heart rate meter 714 that displays the user's current heart rate next to a target heart rate range marker. In an aspect, the heart rate meter 120 can change colors to provide an indication whether the user is above, below or at the target heart rate. In another example, area 716 can provide real-time feedback regarding an amount of calories the user has burned, the current exercise the user is performing, the number of repetitions completed and the current time point in the workout. Further, interface 700 can provide the user with various information sections, such as a goal section 708 noting the user's goals and the goals set by the user's trainer, and a limitation section 718 noting physical limitations of the user.

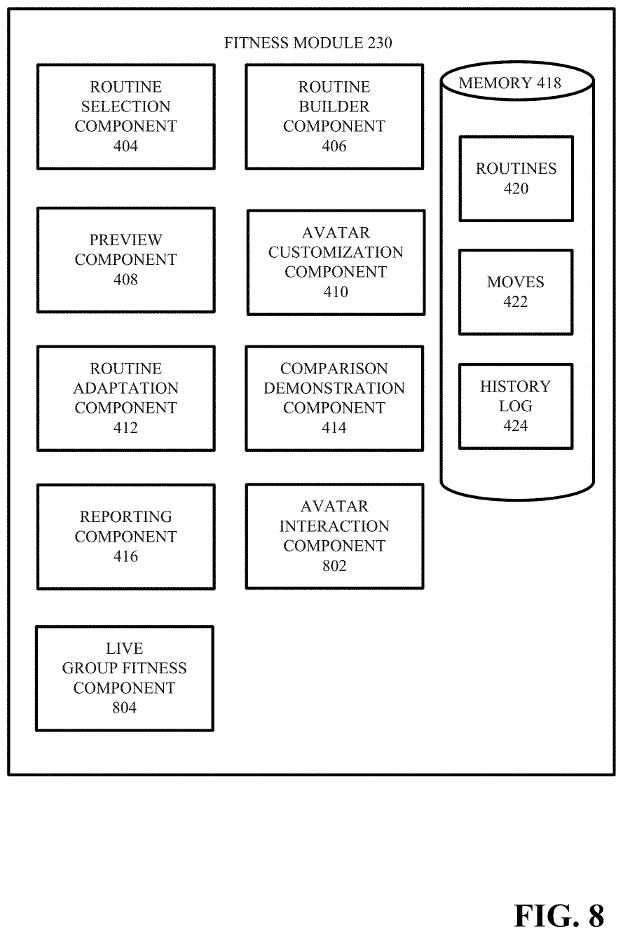

FIG. 8 provides another example embodiment of fitness module 230 in accordance with various aspects described herein. Repetitive description of like elements included in respective embodiments of systems and methods described herein is omitted for sake of brevity.

In accordance with the subject embodiment, fitness module 230 can also include avatar interaction component 802 and live group fitness component 804. Avatar interaction component 802 is configured to facilitate generating and presenting an avatar to the user that is responsive to not only the user's physical state and context but the physical states and contexts of one or more other users. In an aspect, avatar interaction component 802 provides a virtual gaming experience between two or more users. According to this aspect, the two or more users can request to participate in a ‘gaming or interactive mode’ for a specific monitored program, routine, or activity. Each of the users can grant authorization of sharing information (e.g., in real-time or non-real time) regarding their respective performance of the program, routine, or activity between one another's user sessions. Depending on the nature of the program, routine or activity and the gaming or interactive mode selected, any of the user's avatars can react to both feedback about themselves and feedback about the other users.

In particular, avatar interaction component 802 can receive and analyze feedback for a primary user to which an avatar is presented and one or more other user's that are also employing avatar guidance system 200 to perform a similar monitored program, routine or activity. Based on feedback indicating how the other one or more users are performing the monitored program, routine, or activity, avatar interaction component 802 can direct analysis component 212 and reaction component 214 to determine an appropriate reaction for the primary user's avatar. For example, a group of users at various different locations around the world can be involved in a running club. Using avatar guidance system 200 the respective users can set up group runs where they respectively set out to run a same distance together in a race format. For example, each of the users can select the same distance and terrain, start the run at the same time, and have their respective avatars serve as their individual coaches throughout the run. However, not only can the respective avatars respond and react to their respective user's feedback (e.g., as discussed herein), but the respective avatars can also react to feedback received for any of the participating user's. For example, assuming the respective user's names are Abby, Bobby, Carmen, Donna, and Erin. Erin's avatar can tell Erin when to speed up, when to slow down, how to breath, etc. based on physical and physiological activity feedback received for Erin and Erin's personal profile and health information. In addition, Erin's avatar can tell Erin when to speed up and when to slow down based on how Abby, Bobby, Carmen, and Donna are performing when the goal is to win the race. For example, Erin's avatar can note where the other users are in terms of position, speed, fatigue, cramping, etc. Based on this information for example, Erin's avatar can tell Erin to slow down and save some energy for the next mile because she is way ahead of the pack.

It should be appreciated that the above noted group race running example is merely one application of avatar interaction component 802. For example, avatar interaction component 802 can facilitate real-time interaction between two or more users participating together in a variety of different fitness programs routines or activities whether the group participation is competitive in nature (e.g., a tennis match, a boxing match) or collaborative in nature (e.g., a soccer match where the group of users are on the same team).

Group fitness component 804 is configured to provide a group fitness atmosphere for two or more users of avatar guidance system 200 that have chosen to participate in a group fitness routine. The routine is considered a group fitness routine because the two or more users have selected to perform the same routine at the same time (or substantially the same time) and the two or more users have authorized sharing of one or more aspects of their personal avatar experiences with one another during performance of the group fitness routine.

In an aspect, group fitness component 804 can facilitate generation of a shared user interface that is presented to the respective user's participating in the group fitness routine. In one aspect, the interface can include avatar's representative of the respective users in the group. As the respective user's perform the fitness routine, their respective avatars can mimic what the real users are doing (e.g., their movement, facial expression, sounds, etc.). Thus, the interface presented to any one of the respective users can resemble that of a real and live group fitness classroom (or other share environment) in which all the users are present together and performing the fitness routine together.

In another aspect, group fitness component 804 can facilitate generation of a shared trainer or coach to facilitate the group fitness routine. According to this aspect, each of the participating users can be presented with the same avatar coach or trainer. This avatar can respond to user feedback for each of the respective users in accordance with the aspects described herein. However, the responses provided by the avatar based on any one of the users' feedback are collectively received by the group. For example, the avatar trainer can instruct one of the group fitness participants to pick up the pace and then demonstrate a movement to encourage the participant to pick of the pace. This avatar response directed to one of the participants can be seen and heard by each of the participants in a live manner. Similarly, using avatar interaction component 802, the shared trainer can respond to one of the group member's feedback on feedback from other users participating in the group fitness class. For example, the trainer can point out the perfect form exhibited by one of the group fitness members to the other group fitness members. In another example, the avatar trainer can tell a first group fitness member to kick his legs higher (based on the particular fitness move) like a second group fitness member is doing.

In another aspect, group fitness component 804 can provide for a shared trainer experience wherein each of the participants are monitored by the same avatar respectively presented thereto and see and hear the same avatar reactions in a live fashion. However, rather than reacting to individual users, group fitness component 804 can direct routine adaptation component 412 to dynamically modify the routine based on collective feedback from the users as a group. For example, in response to a determination that the majority of the users in the group are becoming extremely fatigued, the routine adaptation component 412 can adapt the group fitness routine accordingly. In response, the shared avatar trainer can instruct the group regarding the adaptation of the routine.

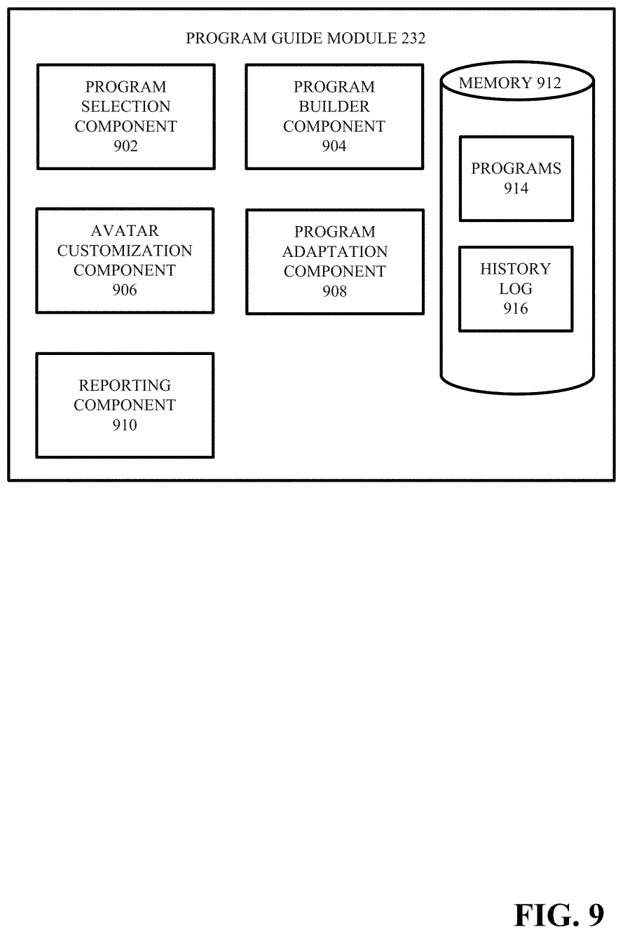

FIG. 9 provides an example embodiment of program guide module 232 in accordance with various aspects described herein. Repetitive description of like elements included in respective embodiments of systems and methods described herein is omitted for sake of brevity.

Program guide module 232 is specifically configured to facilitate guiding or assisting a user with adhering to a program using an avatar in association with various aspects of avatar guidance platform 202. In particular, program guide module 232 is configured to guide a user with performing a program having a plurality of predetermined actions or behaviors for the user to perform (or not perform) over a period of time to achieve a goal of the user. The period of time and goal can vary. However, program guide module 232 is particularly suited for guiding a user through a program that lasts several hours, days, weeks or even months wherein user input 234 is received over the course of the program. For example, a suitable program can include a health and fitness program designed to cause the user to get in better shape and health over a period of three weeks. In another example, a suitable program can include a stress reduction program designed to help a user reduce and combat stress throughout her day. Other examples of suitable programs include an addiction recovery program, a weight loss program, an anger management program, and any conceivable program that includes performance or non-performance of specific user behaviors wherein adherence can be monitored based on analysis of received physical and physiological activity information for the user and/or context information for the user.

During performance of a monitored program, information regarding one or more of the user's physiological state, body movement/position, appearance, and context (e.g. location, time of day, people near the user), is dynamically received and analyzed in view of known requirements of the monitored program, and in some aspects user profile information, to determine or infer whether, how and to what degree the user deviates and/or is likely to deviate from the monitored program (in accordance with aspects described herein). Based on this analysis, reaction component 214 determines or infers a suitable reaction for manifestation by an avatar presented to the user and the avatar control component 208 and avatar generation component 226 causer the avatar presented to the user to perform the reaction. The reaction is configured to provide the user with guidance and/or motivation with adhering to the program and can include visual and audible reactions in the avatar (e.g., speaking, moving, facial expression, tone of voice, etc.). In some aspects, the reaction can include initiation of electronic communication (e.g., sending a notification, initiating an emergency call), and provision of external media (e.g., images, articles, a map, videos, songs, etc.) to the user to facilitate guiding the user with adherence to the program.

For example, a user can select a health and fitness program to perform and have monitored by avatar guidance platform 202. The health and fitness program can include a specific fitness and diet regimen that the user is to follow for a period of 21 days. Over the course of the program, reception component 204 can receive information indicating what food and drink the user has consumed (e.g., as input directly by the user, as provided/determined via various biometric sensors input, as determined via image analysis of food eaten by the user, etc.) and when the user has consumed it. Reception component 204 can also receive information regarding how many calories the user has consumed and burned, user activity/motion levels, user sleep patterns, and physical performance of fitness activities. Reception component 204 can also receive information related to the user's context (e.g., where the user is located, what people, places, things and events are associate with the location, time of day, how the user is feeling, etc.). In addition, information regarding the user preferences, physical health, physical measurements (e.g., height, weight, BMI, specific body part measurements/dimensions (e.g., waist, chest, upper arm/bicep, thigh, etc.), body fat, body composition, muscle composition, muscle density, etc.), performance history, schedule, etc., can be included in an accessible profile for the user.

According to this example, based on the dynamically received user input and the user profile information, analysis component 212 can determine when the user is deviating or is about to deviate from the health and fitness program. For example, analysis component 212 can determine or infer whether the user is eating the required/appropriate food and drink, whether the user is about to make bad food choices, whether the user burned/is burning the required amount of calories in a workout, whether the user performed/is performing a required workout, whether the user is performing the required workout in accordance with requirements for the workout, etc. Based on such determinations and/or inferences regarding whether, how and to what degree the user is deviating or is about to deviate from the requirements of the health and fitness program, an avatar presented to the user can react in a manner to keep the user on track with the program.

Accordingly, the avatar can function as the user's personalized guide configured to follow the user and track the user's behavior and physical state over the course of program and help the user adhere to the program. To provide for various aspects of avatar guidance system 200 when applied to facilitate a guiding a user through a program, program guide module 232 can include program selection component 902, program builder component 904, avatar customization component 906, program adaptation component 908 and reporting component 910. Program guide module 232 can also include various data objects stored in memory 912 (which can be inclusive of memory 218) that are specific to aspects of program guide module 232. These data objects can include information defining various programs 914 capable of being monitored by program guide module 232 and historical information for respective users logging their performance of various programs 232 using program guide module.

Similar to routine selection component 404 that provide for user selection of various preconfigured fitness routines for a user to select for performance, program selection component 902 provides for user selection of a program for performance by the user from a database of known programs 914 and associated requirements. For example, programs database 914 can include a plethora of different programs arranged by various categorical types (e.g., health and fitness, weight loss, weight gain, marathon training, low sugar diet plan, stress reduction plan, sleep regulation programs, addiction recovery program, etc.). In an aspect, each of these programs can include a description of the requirements of the program and the goals of the program. Each of these programs can be designed for performance over a fixed period of time or a boundless amount of time. For example, a user can select a fitness program designed as a 90-day challenge or select a stress reduction program or alcohol addiction recovery for performance on a daily basis until the user chooses to stop the program.

Similar to routine builder component 406 which facilitates building of a custom fitness routine by a user (or supervisor of the user), program builder component 904 facilitates designing a custom program. In an aspect, a user can select a preconfigured program and change certain variables/requirements of the program to fit the user's needs. In other aspects program builder component 904 can allow a user (or a supervisor of the user) to build a custom program from scratch. For example, a user can set a schedule the user would like to adhere to, defining what the user should do and when and how the user should respond to certain scenarios.

In another aspect, program builder component 904 can automatically adapt a preconfigured program to fit the user's needs based on profile information for the user (e.g., user preferences, user health history, user physical abilities, user habits etc.) or program builder component 904 can build a custom program (e.g., from scratch) for a user based on the user's goals and other profile information for the user (e.g., preferences, demographics, location, language, user, budgetary constraints, habits, health history, physical appearance and measurements, dietary restrictions, medications, schedule, and/or historical performance information (regarding past programs, routines, or activities tracked via avatar guidance system 200). For example, a user can state that her goal is to lose 10 pounds in one month. Based on various information about the user's preferences (e.g., what foods the user likes/dislikes, what types of physical exercise the user likes/dislikes, how much and when the user likes to work out, etc.), demographics, location, language, user, budgetary constraints, habits, health history, physical appearance and measurements, dietary restrictions, medications, schedule, and/or historical performance information, program builder component 904 can generate a custom health and fitness program that is designed to cause the user to achieve her goal if followed properly. Program builder component 904 can also employ routine builder component 906 to facilitate generating custom fitness routines to include in a health and fitness program.

In an aspect, the user can analyze the custom program and provide input regarding modifications to the program. For example, a health and fitness program output by program builder component 904 could include some requirements that the user would like to change. According to this example, the program could require the user to work out 5 days a week when the user would prefer to work out 4 days a week. Based on input requesting the program to be modified to working out 4 days a week, program builder component 904 can automatically adjust other requirements of the program to maintain the user's goal. For example, program builder component 904 could adjust the intensity of the workouts and/or modify her diet to account for the lost workout day. In other aspect, program builder component 904 can request specific user input to facilitate creating a custom program for the user. For example, depending on the type of program and the user's goal, program builder component 904 can have the user fill out a form (e.g., or otherwise provide input) with answers to a specific set predetermined questions associated with the creation of the type of program. The user's answers can be used to create/customize the program for the user.

In an aspect, in association with generation of a custom health and fitness program for a user, the user can provide program builder 904 with an image of the user and/or avatar guidance platform 202 can receive captured image data of the user (e.g., via visual capture device 110). For example, two or three-dimensional image data can be captured and received for a user. The captured image data can be provided to avatar guidance platform 202 in the form of a two or three-dimensional visual replica/representation of the user or avatar guidance platform 202 can employ the captured image data to generate a be a visual replica/representation of the user (e.g., a two or three-dimensional avatar representation of the user). In addition to image data, physical measurements (e.g., regarding user height, weight, dimensions of body parts, BMI, etc.) can be received for the user and/or detected via the image data. This measurement data can facilitate generation of a two or three-dimensional visual representation of the user.

According to this aspect, the user can provide input regarding characteristics of body features that the user would like to change and how the user would like them to change using the image of the user and/or the visual representation of the user. For example, the user could circle or target certain body parts and indicate how the user would like them to change. According to this example, the user could point to her stomach and indicate she would like to trim off inches from her waist or develop a six pack. In another example, the user could point to her arms and indicate she would like to increase the diameter of her biceps. In an aspect, this input can be received from the user by directly allowing the user to draw on/mark up the image/visual representation of the user. For example, the user could place lines or marks on parts of the user's body indicating how the user would like the body parts to appear.

Based on received input indicating characteristics of body features that the user would like to change and how the user would like them to change based on user input regarding an image or visual replica of the user, user profile information, and any other user input regarding requirements for the program, (e.g., duration of the program, preferences for the program, exercises and foods the you would like included/excluded from the program, etc.), program builder component 904 can design a custom diet and/or exercise program for the user. The custom diet and exercise program is specifically tailored to achieve the user's goals (e.g., the changes to the user's physical appearance as pointed on by the user with respect to an image or visual representation of the user).

In another aspect, in association with generation of a custom health and fitness program for a user, the user can provide program builder 904 with an image or visual representation of physical features the user desires. For example, the user could provide program builder component 904 with a picture of an athlete and request to have a program designed for the of the user that will cause the user to look like the athlete. In another example, the user could provide program builder component 904 with an image of a supermodel and indicate that she would like to transform her body to look like the supermodel. In another example, the user could provide program builder component 904 with a picture of a specific body part and request a program that would help the user transform his or her corresponding body part to appear like that in the picture. For example, the user can provide program builder component 904 with an image of a person's shoulders and indicate he would like his shoulder to look like the picture.

According to this aspect, based on received input indicating characteristics of body features that the user would like to have based on image data representing those characteristics, user profile information, and any other user input regarding requirements for the program, (e.g., duration of the program, preferences for the program, exercises and foods the you would like included/excluded from the program, etc.), program builder component 904 can design a custom diet and/or exercise program for the user. The custom diet and exercise program is specifically tailored to achieve the user's goals (e.g., the changes to the user's body features to have the characteristics of those provided in the image data.

Similar to avatar customization component 410, avatar customization component 906 can provide for customizing the appearance and personality of an avatar selected by the user to guide the user through a program. For example, the user can choose the gender, age, ethnicity, language, clothing, voice, and personality of their avatar. The user can also choose an avatar from a database of preconfigured avatar's having various appearances and personalities. In an aspect, the avatar can embody a persona selected by the user, such as the user's conscience (e.g., the voice of reasoning or angel on the user's shoulder), the user's friend, the user's mother, the user's coach/trainer, the user's doctor, the user's therapist, etc. In another aspect, the user can select multiple avatars to help guide the user with different aspects of a program. For example, with respect to a diet and exercise program, the user can select a first avatar to facilitate adherence to aspects of the program related to the user's diet and a second avatar to facilitate aspects of the program related to the user's exercise.

Similar to routine adaptation component 412, program adaptation component 908 can facilitate dynamically adapting aspects and requirements of a program during the course of the program. Program adaptation component 908 can adapt aspects and requirements of a program based on a variety of aspects including but not limited to: user physical and physiological activity data and context data, reference data for the user physical and physiological activity data and context data for the program, and user profile information (e.g., user preferences, user goals, past performance history, etc.). For example, program adaptation component 908 can adjust and/or dynamically determine new or modified requirements of a program based on a determination that the requirements are too difficult or too easy because the user is consistently not meeting or exceeding the requirements of the program, respectively.

In another example, program adaptation component 908 can analyze a user's history of performance with respect to adherence to a program and dynamically adapt the requirements of the program to account for the user's past performance. For example, where a user ran an extra 5.0 miles on the workout the day before, program adaptation component 908 and/or routine adaptation component 412 can change the requirements of today's workout to account for the extra 5.0 miles from the day before. For example, where today's workout was supposed to be a longer run, program adaptation component can determine that the user's goals (e.g., to get in shape for an upcoming race) would be better suited if the workout today was switch to yoga (e.g., to prevent injury due to over exercise). In another example, program adaptation component 908 can adapt current requirements of a user's program based on how the user is feeling (e.g., sore, sick, energized, sad, tired, etc.), an amount of sleep received, an amount of activity of the user that day/week, an amount of stress the user is experiencing, what the user's current physiological state is, what the user's current context is, what the user ate that day (e.g., ate an extra number of calories, earn an extra number of jumping jacks), unexpected changes to the user's schedule, unexpected injuries, events in the user's life, how the user is progressing in the program, resources available to the user (e.g., group meetings, therapy, exercise equipment, food options, etc.).

In another example, the program adaptation component 908 can regularly (e.g., once an hour, twice a day, once a day, once a week, etc.) evaluate a user's progress through a program in view of the user's goals and determine whether and how to adapt the requirements of the program based on the user's progress. For example, when a user's is hoping to lose X amount of weight and the user is not on target to achieve this goal if the user adheres to the current requirements of the program for the remainder of the program, program adaptation component 908 can adjust the dietary and/or exercise requirements of the program to ensure the user will achieve her goal.

Similar to reporting component 416, reporting component 910 can send information regarding a user's performance of a program in real-time or substantially real-time to another entity (e.g., another user, another system) for interpretation thereof. For example, with respect to drug rehabilitation program, reporting component 910 can send information regarding a user's adherence or deviation from the program to the user's therapist in real-time. In another example, with respect to a diet and exercise program, reporting component can send information regarding the user's performance of the program to the user's real life (e.g., human) trainer in real-time.

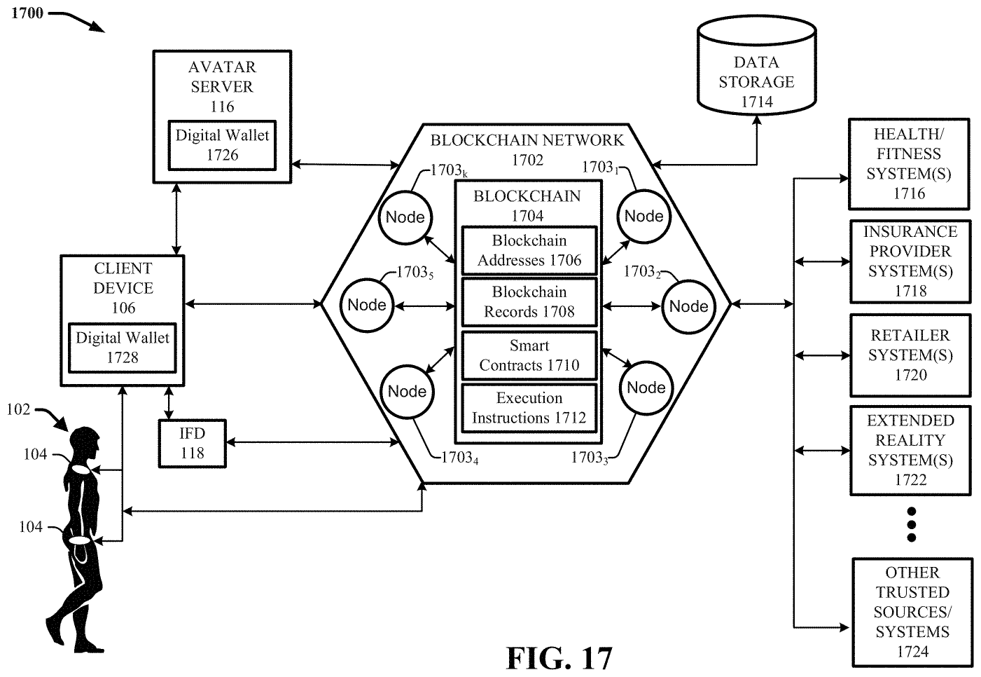

In accordance with system 100, system 200 (e.g., avatar guidance system 200) and additional systems described herein, (e.g., system 1300 and/or system 1400), users can perform fitness activities (e.g., fitness activities, routines, etc.) in association with being monitored via an avatar guidance platform that can track user performance of fitness activities while providing guidance and feedback regarding the user's physical and physiological performance based on analysis of sensory feedback captured for the user (e.g., captured via one or more sensors 104 worn by the user and/or integrated withing an IDF 118) and/or image data captured of the user. In this regard, the avatar server 116 can receive and/or determine and track a variety of health and fitness information for users. In various embodiments, this health and fitness information can be logged for the respective users (e.g., via reporting component 416 and/or reporting component 910 in history logs for the respective users (e.g., history log 424, history log 916 and the like). In other embodiments, as discussed in greater detail infra with reference to FIG. 17, this health and fitness information (and/or defined events or subsets of the health and fitness information) can be automatically reported to and recorded on one or more blockchain networks.

In this regard, the health and fitness information can include information regarding completion of fitness activities, type of fitness activities completed, timing of initiation and completion and duration of fitness activities. The avatar system can also receive and/or determine and track performance metrics regarding physiological parameters of the user during the workout (e.g., heart rate, body temperature, respiration, perspiration, blood pressure, calories burned, etc.) and relative to individual exercise moves. The avatar system can also receive and/or determine and track information regarding specific exercise moves performed, number of reps, proper and improper form and motion, level of exertion, amount of weight used, information regarding physical fitness practices and abilities of the user (e.g., how fast the user can run, how high the user can jump, how much weight the user can lift, flexibility of the user, range of motion of the user, etc.). For example, the health and fitness information can include information regarding performance of fitness activities, including information regarding activity type, exercises performed (e.g., types of moves performed, types of equipment used), and performance metrics associated with the activities and/or exercises performed (e.g., regarding amount, frequency, duration, level, timing, intensity, form correctness, calories burned, heart rate, etc.). The health and fitness information can also include information regarding location, distance and route where applicable (e.g., for running, biking, swimming, rowing, etc. activities that involve movement over distances).

The health and fitness information can also include information regarding physical measurements of a user (e.g., height, weight, BMI, specific body part measurements (e.g., waist, chest, upper arm/bicep, thigh, etc.), body fat, body composition, muscle composition, muscle density, etc.) generated in an automated manner via one or more via one or more devices. For example, as described with reference to system 100 (and additional systems described herein), in some embodiments, this information can be captured and reported to the client device 106 and/or the avatar server 116 via one or more sensors 104 and/or one or more IFDs 118 (e.g., smart scales, smart mirrors). Additionally, or alternatively, physical measurement data can be determined by the client device 106 and/or the avatar server 116 in an automated manner based on analysis of image data captured of the user. For example, image data for a user captured by visual capture device 110 can be analyzed using two-dimensional and/or three-dimensional image analysis to determine physical measurements metrics for the users, such as height, weight, body fat, dimension of the user's waist, hips, shoulders, biceps, etc., as a user's body changes over time (e.g., in response to performance of an exercise or diet program).

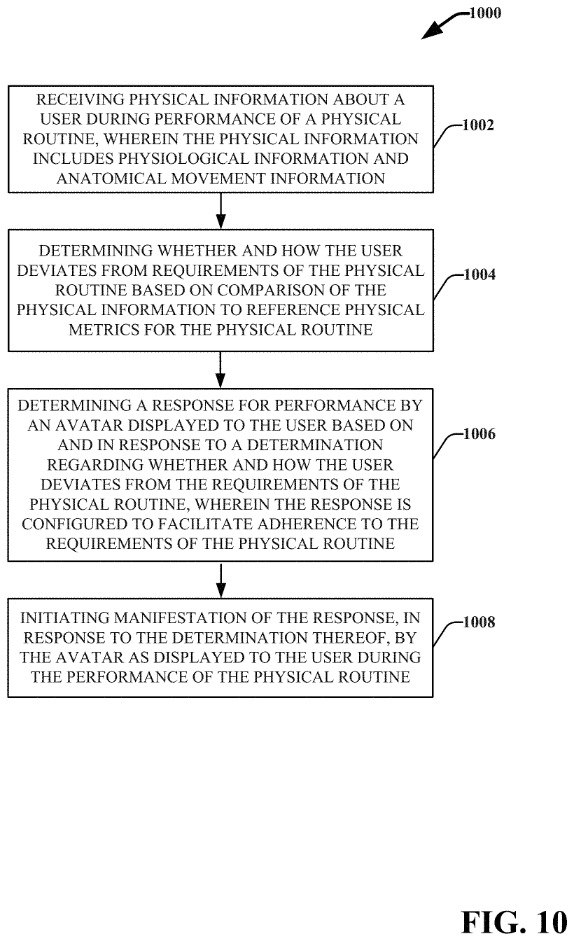

In view of the example systems and interfaces described herein, example methods that can be implemented in accordance with the disclosed subject matter can be further appreciated with reference to flowcharts in FIGS. 10-12. For purposes of simplicity of explanation, example methods disclosed herein are presented and described as a series of acts; however, it is to be understood and appreciated that the disclosed subject matter is not limited by the order of acts, as some acts may occur in different orders and/or concurrently with other acts from that shown and described herein. For example, a method disclosed herein could alternatively be represented as a series of interrelated states or events, such as in a state diagram. Moreover, interaction diagram(s) may represent methods in accordance with the disclosed subject matter when disparate entities enact disparate portions of the methods. Furthermore, not all illustrated acts may be required to implement a method in accordance with the subject specification. It should be further appreciated that the methods disclosed throughout the subject specification are capable of being stored on an article of manufacture to facilitate transporting and transferring such methods to computers for execution by a processor or for storage in a memory.

FIG. 10 illustrates a flow chart of an example method 1000 for generating a personalized avatar that is responsive to a user's physical state and context, in accordance with various aspects and embodiments described herein. At 1002, information about a user is received during performance of a physical routine by the user (e.g., via reception component 204). The information includes physiological information and anatomical movement information. For example, as a user performs a workout routine or a particular sports activity, information regarding the user's heart rate, blood pressure, perspiration, cortisol level, glucose level, etc. can be received. In addition, information regarding what movements the user is performing, how the user's body is positioned during the movement and intensity of the movement can be received. At 1004, it is then determined whether and how the user deviates from requirements of the physical routine based on comparison of the information to reference physical and physiological activity metrics for the physical routine (e.g., by analysis component 212). For example, analysis component can determine that the user's physiological state indicates that the user does not comply with what the user's physiological state should be for a particular exercise of the physical routine. In another example, analysis component can determine that the user's body position during a particular exercise yoga exercise is on target or not on target with how it should be.

At 1006, a response is determined for performance by avatar displayed to the user, based on and in response to, a determination regarding whether and how the user deviates from the requirements of the physical routine (e.g., via reaction component 214). The response is configured to facilitate adherence to the requirements of the physical routine. For example, reaction component 214 can determine a response that includes provision of spoken instruction and physical demonstration by the avatar regarding an action for the user to perform that corrects a deviation. According to this example, where the user's pace and intensity of an exercise move is below the required level, the avatar response can include act of performance of the exercise move at the required intensity level with exclamatory words of encouragement spoken in an enthusiastic tone telling the user to “pick up the pace, let's go let's go!” Once the user corrects the deviation, the avatar's behavior can change accordingly (e.g., the avatar can go back to evaluating the exercise as opposed to demonstrating, and the avatar can congratulate the user for adhering to the requirements of the fitness routine). At 1008, manifestation of the response is initiated, in response to the determination thereof, by the avatar as displayed to the user during the performance of the physical routine (e.g., by the avatar control component 208, and the avatar generation component 226). In other words, the avatar presented to the user is caused to perform the response.

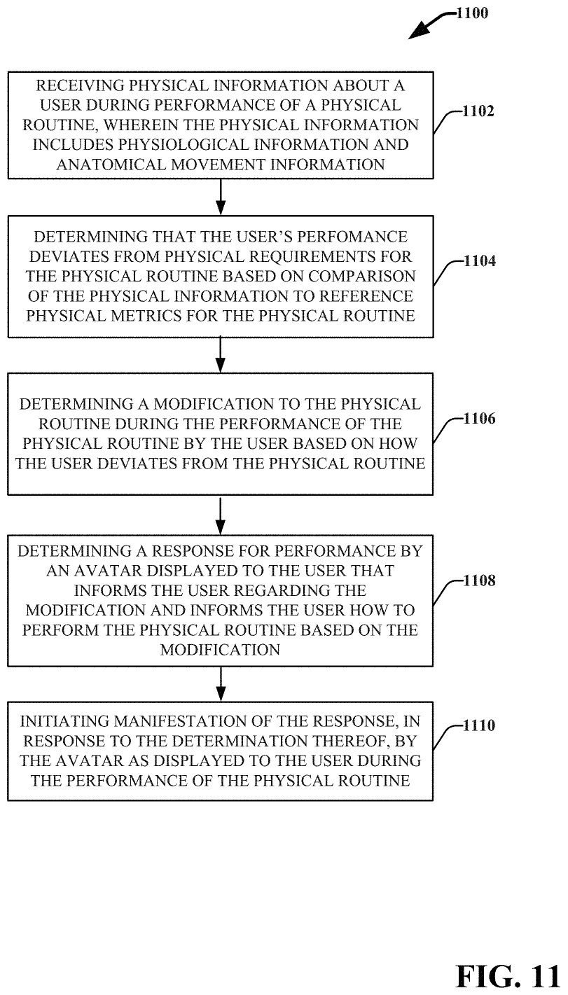

FIG. 11 illustrates a flow chart of another example method 1100 for generating a personalized avatar that is responsive to a user's physical state and context, in accordance with various aspects and embodiments described herein. At 1102, information about a user is received during performance of a physical routine by the user (e.g., via reception component 204). The information includes physiological information and anatomical movement information. For example, as a user performs a workout routine or a particular sports activity, information regarding the user's heart rate, blood pressure, perspiration, cortisol level, glucose level, etc. can be received. In addition, information regarding what movements the user is performing, how the user's body is positioned during the movement and intensity of the movement can be received. At 1104, it is then determined that the user's performance deviates from physical and physiological activity requirements for the physical routine based on comparison of the information to reference physical and physiological activity metrics for the physical routine (e.g., by analysis component 212). For example, analysis component can determine that the user's physiological state indicates that the user is consistently (with respect to a deviation frequency threshold) falling below physical intensity requirements for the routine (e.g., based on heart rate, perspiration, fatigue, range of motion, etc.).

At 1106, a modification to the physical routine is determined during the performance of the physical routine by the user based on how the user deviates from the physical routine (e.g., by routine adaptation component 412). For example, the routine adaptation component can determine that the user should change the required exercises to an easier option. At 1108, a response for performance by an avatar displayed to the user is determined that informs the user regarding the modifications and informs the user how to perform the physical routine based on the modification (e.g., via reaction component 214). The response is configured to facilitate adherence to new requirements of the modified physical routine. At 1118, manifestation of the response by the avatar is initiated in response to the determination thereof, by the avatar as displayed to the user during the performance of the physical routine (e.g., by the avatar control component 208, and the avatar generation component 226).

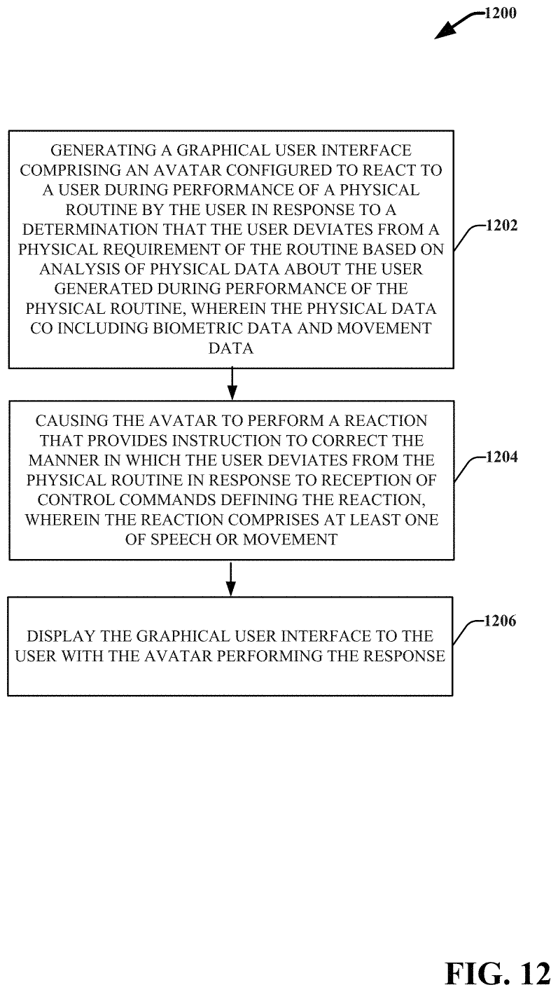

FIG. 12 illustrates a flow chart of another example method 1200 for generating a personalized avatar that is responsive to a user's physical state and context, in accordance with various aspects and embodiments described herein. At 1202, a graphical user interface is generated that includes an avatar configured to react to a user during performance of a physical routine by the user in response to a determination that the user deviates from a physical requirement of the routine based on analysis of biometric and movement data about the user generated during performance of the physical routine (e.g., via interface component 206). At 1202, the avatar is caused/directed to perform a reaction that provides instruction to correct the manner in which the user deviates from the physical routine in response to reception of control commands defining the reaction, wherein the reaction comprises at least one of speech or movement (e.g., via avatar generation component 226). At 1206, the graphical user interface is displayed to the user with the avatar performing the response (e.g., via rendering component 226).

### IV—Example Avatar Visualization System

In one or more aspects, a system is provided that includes a reception component configured to receive information corresponding to a user's physical appearance and physical health, an analysis component configured to determine or infer one or more changes to the user's physical appearance based on predicted performance of a health and fitness program by the user and the user's physical health, and a visualization component is configured to generate a visual representation of the user based on the information and the one or more changes to the user's physical appearance.

In another aspect, a method is disclosed that includes receiving information corresponding to a user's physical appearance and physical health, predicting one or more changes to the user's physical appearance based on predicted performance of a health and fitness program by the user and the user's physical health, and generating a visual representation of the user based on the information and the one or more changes to the user's physical appearance.

In yet another aspect, a method is provided that includes using a processor to execute the computer executable instructions stored in a memory to perform the various acts. These acts include receiving information corresponding to a user's physical appearance and physical health, predicting one or more changes to the user's physical appearance based on predicted performance of a health and fitness program by the user and the user's physical health, generating a visual representation of the user based on the information and the one or more changes to the user's physical appearance, and displaying the visual representation via a graphical user interface.

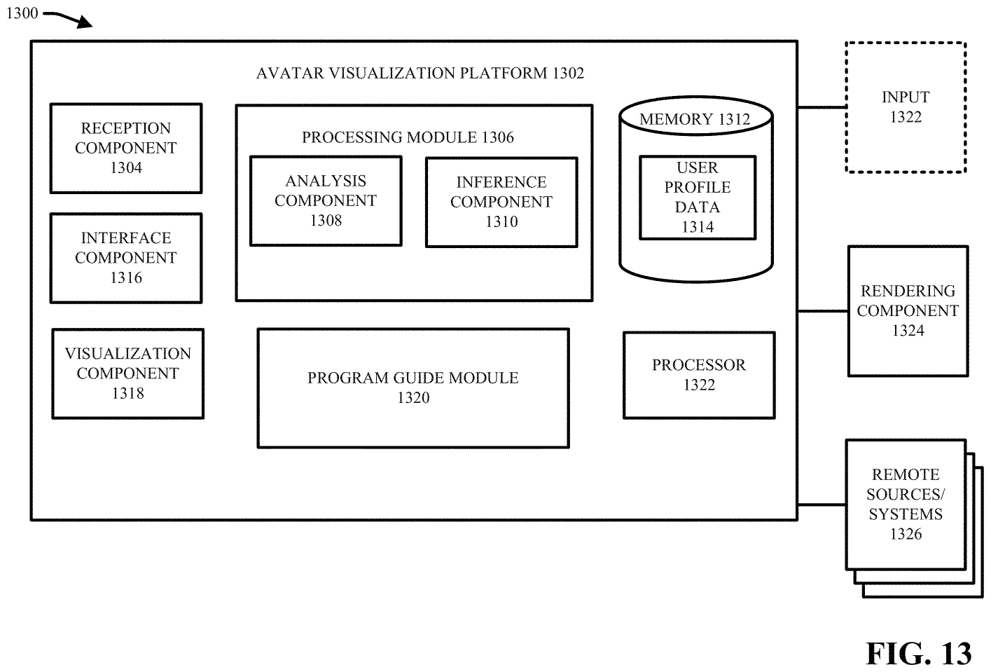

FIG. 13 presents an example avatar visualization system 1300 that facilitates visualizing how the user will appear in response to adhering to a health and fitness program in accordance with various aspects and embodiments described herein. System 1300 can include same or similar features and functionalities as system 100. In an aspect, system 1300 employs the architecture of system 100, including one or more of the various components/devices of system 100, wherein avatar visualization platform 1302 corresponds to avatar platform 114. Avatar visualization platform 1302 can thus include same or similar features and functionalities as avatar platform 114. In various embodiments, the avatar platform 114 can include avatar visualization platform 1302 and avatar guidance platform 202, or portions of the respective platforms. Repetitive description of like elements employed in respective embodiments of systems and interfaces described herein are omitted for sake of brevity.

Avatar visualization system 1300 can include avatar visualization platform 1302, input 1322, rendering component 1324 and one or more remote sources/systems 1326. Input 1322, rendering component 1324 and the one or more remote sources/systems 1326 can respectively correspond to input 234, rendering component 236 and one or more remote sources/systems 238 discussed with respect to FIG. 2. Avatar visualization platform 1302 is configured to generate a visual representation or replica (e.g., an avatar) of a user based on received appearance information for the user, received appearance data for the user, and known health information for the user (including physical characteristics of the user and physiological characteristics of the user). In particular, avatar visualization platform 1302 can generate an avatar that is a visual replica of the user as the user currently appears. Accordingly, as the user's appearance changes over the course of performance of a health and fitness program, the appearance of the avatar can also change to provide a mirrored visualization of the changes in the user. For example, each new day of the program, the appearance of the user can be evaluated, captured and imparted to the avatar. Thus in essence, the user's avatar can resemble a mirror image of himself over the course of a program.

Avatar visualization platform 1302 is further configured to generate a visual representation of the user that is a prediction of how the user will appear at a future point in time based on performance of a health and fitness program by the user. For example, the user can select, (and/or design, or otherwise be assigned) a specific health and fitness program for performance using fitness module 1302 (wherein fitness module 1302 can include same or similar features and functionalities of fitness module 230). Using avatar visualization platform 1302, the user can be provided a visual representation that demonstrates how the user will predicatively look after performance/completion of the program if the user adheres to the requirements of the program. In addition, the user can select various time points in the program (e.g., after week 1, after week 2, after week 3, etc.) and avatar visualization platform 1302 can generate a visual representation of the user that demonstrates how the user will predicatively look at the respective time points if the user adheres to the requirements of the program.

This feature of avatar visualization platform 1302 allows a user to dynamically pick and choose different health and fitness programs and/or change different variables of a health and fitness program and see how the user would appear in the future based on the selected health and fitness program and/or the different variables. Accordingly, a user can select a health and fitness program that will cause the user to achieve an optimally desired appearance. For example, as the user selects different health and fitness programs and/or can changes variables of a selected health and fitness program, avatar visualization platform 1302 can dynamically adapt the appearance of an avatar presented to the user that corresponds to a predicted visualization of how the user will appear based on completion and adherence to the different health and fitness or the health and fitness program with the respectively chosen variables. As a result, the user can select a health and fitness program based on how it will affect the user's appearance.

In an aspect, the avatar is generated and presented to the user via a rendering component 1324 located at a client device (e.g., client device 106). Rendering component 1324 can include suitable hardware (e.g., a display screen, a hologram generation device, etc.) and software (e.g., software for generating a GUI and/or software for accessing and rendering a network-based interface, such a browser) to accomplish generating and presenting an avatar. Generally, avatar visualization platform 1302 can include memory 1312 that stores computer executable components and processor 1322 that executes the computer executable components stored in the memory, examples of which can be found with reference to FIG. 35.

It is to be appreciated that although avatar guidance visualization platform 1302 is illustrated as being a standalone component, such implementation is not so limited. For example, avatar visualization platform 1302 can be located at a client device (e.g., client device 106), a remote server (e.g., avatar server 116) or the cloud. In another aspect, one or more components of avatar visualization platform 1302 can be located at both the client device and the remote server. In yet another aspect, the components of avatar guidance platform can be distributed between the client and the server. Still in yet another aspect, avatar visualization platform 1302 can be included within avatar guidance platform 202, or vice versa. All or portions of avatar visualization platform 1302 can be operatively coupled to all or portions of avatar guidance platform 202, or vice versa. Moreover, avatar visualization platform 1302 can be granted access to all or portions of avatar guidance platform 202, or vice versa.

Avatar visualization platform 1302 can include reception component 1302, processing module 1306, interface component 1316, visualization component 1318 and program guide module 1320. Reception component 1302 is configured to receive various inputs 1322 including the data noted with respect to input 234. In accordance with aspects of avatar visualization platform 1302, input 1322 particularly includes a variety of information about the user's physical health and appearance. In an aspect, this physical health and/or appearance information is provided by the user (or another user such as the user's medical caregiver or therapist) or extracted from a remote system/source (e.g., electronic medical/health records for the user provided by a medical institution). In another aspect, physical health and/or appearance information for a user is captured via various sensing devices 106 worn by or otherwise attached to the user, intelligent fitness device(s) employed by the user (e.g., intelligent fitness devices 118), other remote medical/sensing equipment employed by the user (e.g., a heart monitor, a scale, etc.), and/or a visual capture device (e.g., visual capture device 110) pointed at the user. In an aspect, health and/or appearance information is received by reception component 1304 during a capture process prior to generation of a visual replica of the user. In another aspect, this physical health and/or appearance data can be received for the user in real-time as the user performs a fitness routine or health and fitness program.

In an aspect, physical health and appearance information received for a user is stored by avatar visualization platform 1302 in association with a user profile established for the user (e.g., user profile data 1314). User profile data 1314 can also include the various profile information described with respect to user profile data 222. For example, a user can establish a profile with avatar visualization platform 1302 that includes information defining the user's preferences (e.g., diet and exercises preferences), health information, demographics and schedule. The profile can also include a logged history of captured physical and physiological activity data for the user over the course of performance of various fitness and/or other programs by the user (e.g., using avatar guidance platform 202), and other logged/monitored information for the user (e.g., habits, food and drink consumption, activities levels, sleep patterns, etc.).

Physical health information for a user can include any information regarding a user's mental and physical health. For example, physical health information can include but is not limited to: physical measurements (e.g., height, weight, BMI) of the user, physical and physiological conditions effecting the user, mental illness, any past or current physical injuries affecting the user, surgeries received by the user, history of medical conditions associated with the user and the user's relatives, any medications taken by the user, and tobacco and alcohol consumption of the user etc. Health information can also include information related to physiological indicators/measures that are capable of being determined for a user using various know clinical laboratory tests, including information related to the user's genetic make-up, protein levels, hormone levels, blood type, white/red blood cell count, metabolism, etc. Health information can also include information regarding physical fitness practices and abilities of the user (e.g., how fast the user can run, how high the user can jump, how much weight the user can lift, flexibility of the user, range of motion of the user, etc.).

Appearance information for a user includes two or three-dimensional image data captured/taken of the user. For example, the user can have image data taken of himself or herself (e.g., a picture, a video, a series of three-dimensional image data captures of the user from different camera perspectives relative to the user, etc.) as the user stands still and/or as the user performs various test/model movements (e.g., using a visual capture device 110). At this time, data regarding the user's physiological state can also be captured by various medical devices or biometric sensing devices (e.g., devices 106) and received by reception component 1304. For example, the physiological data captured and provided to reception component 1304 (e.g., via a wired or wireless connection) can include information regarding the user's blood pressure, perspiration, respiration, white blood cell count, temperature, glucose level, drugs present in the body, antigens present in the body, etc. Other example data that can be captured and reported by a sensing/medical device can include information indicating the user's physical measurements (e.g., height, weight, BMI, etc.).

In an aspect, image data captured for a user is used to generate a visual representation of the user corresponding to the user's current appearance. In one aspect, the visual capture device employed to capture the image data (e.g., visual capture device 110) can generate this visual representation. In another aspect, the visual capture device can provide the captured image data to reception component 1304 and visualization component 1318 can employ the image data to generate the three-dimensional representation of the user as the user currently appears. For example, visualization component 1318 can generate an avatar that resembles the current appearance of the user. Interface component 1316 is configured to generate a graphical user interface that includes the avatar. In an aspect, interface component 1316 can include same or similar functionalities as interface component 206. An interface including an avatar generated by visualization component 1318 is further presented to the user via rendering component 1324.

In an aspect, an avatar generated by visualization component 1318 can further be configured to exhibit the various features and functionalities of avatars discussed herein. For example, an avatar generated by visualization component 1318 that resembles the appearance of a user can function as the user's guide or coach in association with performance of a monitored program, routine or activity, as discussed with respect to avatar guidance system 200.

In addition to generating an avatar that resembles a user's current appearance. Visualization component 1318 is further configured to generate a visual representation of the user that is a prediction of how the user will appear at a future point in time based on performance of a health and fitness program by the user. In particular, processing module 1306 is configured to analyze received input regarding a user's current appearance and determine or infer how the user's appearance will change in response to performance of specific health and fitness program based on the user' current physical appearance, the user's health information (as included in the user's profile), other profile information for the user (e.g., age, gender, ethnicity, etc.), and the requirements of the health and fitness routine.

For example, analysis component 1308 can employ information relating different diet and exercise program, and/or features of the respective programs to measurable appearance metrics including but not limited to: to weight loss, weight gain, fat loss, fat gain, dimensions of body parts, and muscle definition/appearance, with respect to individual human body parts and/or the human body as a whole. Analysis component 1308 can further adjust these appearance metrics to account for specific health information for the user (e.g., physical measurements, physical conditions, surgeries received, medications taken, dietary restrictions, metabolism, and other health information), along with information regarding the user's age, gender, ethnicity, and/or any other personal information that can have an effect on a user's body appearance and function. For example, the manner in which a specific diet and exercise regimen changes the amount of inches lost and muscle definition established with respect to a user's abdominal area can depend on the user's health profile, age and gender.

Analysis component 1308 can thus determine how a particular user's body and appearance will likely change based on a specific diet and/or exercise regimen. Based on these determined changes, visualization component 1318 can generate a visual representation of the user that depicts how the user will look following performance of the specific diet and/or exercise regimen. In an aspect, visualization component 1318 can modify current image data of a user to account for the changes determined by analysis component 1308. For example, visualization component 1318 can generate an avatar depicting the user as the user currently appears and morph the avatar's appearance, based on the changes determined by analysis component 1308, to generate a new visual representation of the user depicting how the user will appear after performance of the diet and exercise regimen.

In some aspects, analysis component 1308 can specifically determine how a user's body will change at different points over the course of a health and fitness program. According to this aspect, a user can request to see a visualization of how he or she will appear at any point during the health and fitness program. For example, when a user has selected a 90-day program, before beginning the program, the user can be provided with a visualization of how he or she would look after completion of the program. The user could also request to see how he or she would look on day 10, on day 32, on day 45, etc., and visualization component can 1318 generate the respective visual representations of the user. According to this aspect, analysis component 1308 can determine how a user's appearance will change based on analysis of the user's current appearance, health information, and age/gender in view of what will be performed by the user (e.g., diet and exercise wise), over the duration of time from the initiation of the program to the selected time marker.

In an aspect, analysis component 1308 can employ inference component to facilitate inferring how the user's appearance will change in response to performance of the specific health and fitness program based on the user' current physical appearance, the user's health information, and the requirements of the health and fitness routine. Inference component 1310 can include same or similar features and functionalities as inference component 216. In an aspect, inference component 1308 analyze appearance information for a plurality of user's regarding how performance of different health and fitness routines and regimes effected changes in their appearance in view of respective health information (e.g., starting height, weight, BMI, age, ethnicity, medications taken, physical conditions, physical abilities, etc.) for the different users. Inference component 1308 can further employ various machine learning techniques to optimize determinations regarding how a specific user's appearance will be affected by different health and fitness programs (e.g., including specific diet and fitness requirements of the different health and fitness programs).

For example, inference component 1308 can analyze historical performance and appearance information for a set of users having a similar health profile, age and gender as a current user. Inference component 1308 can then determine how performance of these different health and fitness programs actually effected changes their respective appearance and employ this information to infer how performance of the different health and fitness routines will affect the current user's appearance. In an aspect, visualization component 1318 can present a user with example images of before and after visualizations/pictures taken of other users who performed the different health and fitness programs.

Avatar visualization platform 1302 can also include program guide module 1320. Program guide module 1320 can include same or similar functionality as program guide module 232. In particular, program guide module 1320 can include program selection component 902, a program builder component 904. Using these respective components, a user can select or design a specific health and fitness program. The program builder component 904 can also employ various features of fitness module 230 (e.g., routine selection component 404, routine builder component 406, etc.) to facilitate selecting or designing specific fitness routines/activities for inclusion in a health and fitness program. The requirements of a fitness program selected/designed by the user can vary but can generally relate to dietary requirements, fitness requirements, and a duration of the program. Based on the health and fitness program selected or designed by the user, visualization component 1318 can generate a representation of the user as the user will appear in response to performance of the program. Accordingly, a user can employ avatar visualization component 1318 in conjunction with fitness module 1320 to facilitate selecting/designing a health and fitness program that will bring about desired changes in the user's appearance.

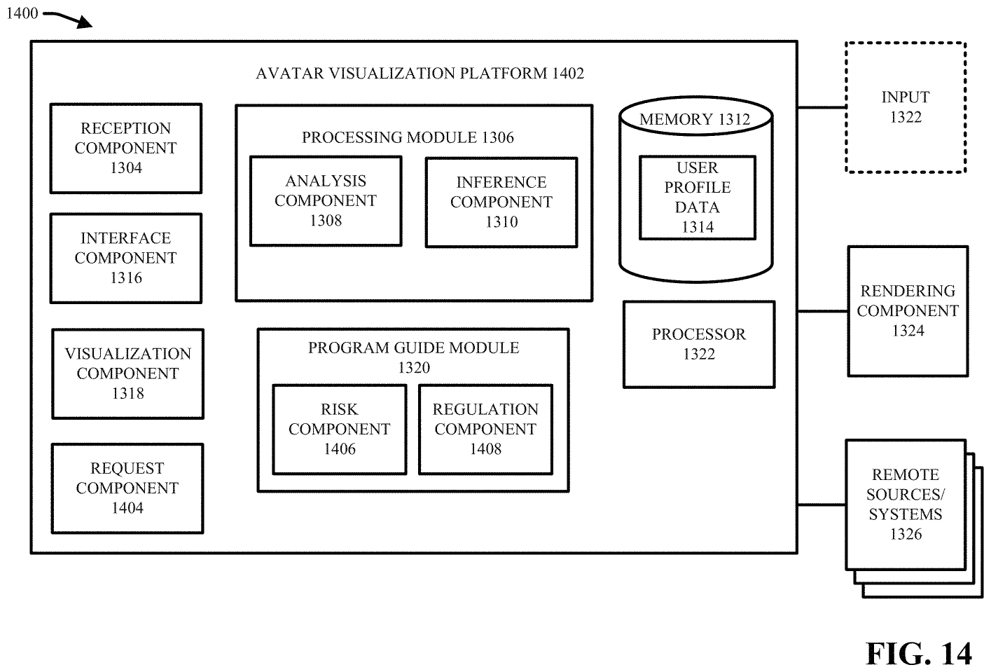

FIG. 14 presents another example avatar visualization system 1400 that facilitates visualizing how the user will appear in response to adhering to a health and fitness program in accordance with various aspects and embodiments described herein. System 1400 can include same or similar features and functionalities as system 1300, with the addition of request component 1404 and risk component 1406 and regulation component 140 to program guide module 1320. Repetitive description of like elements employed in respective embodiments of systems and interfaces described herein are omitted for sake of brevity.

Avatar visualization platform 1402 is configured to facilitate designing a health and fitness program to achieve a desired visual appearance of the user. In an aspect, a user can provide a request via request component 1404 that includes information identifying one or more characteristics of the user's body that the user would like to achieve. Based on the request, the user's current appearance, and the user's health and physical/physiological state information, program guide module 1320 (e.g., via program builder component 904), can generate a personalized diet and exercise program for the user that is designed to cause the user's appearance to change in the requested manner.

For example, the user could submit a request via request component 1404 indicating how she would like her stomach to appear, her hips to appear, her thighs to appear, etc. The request can also indicate how much the weight the user would like to lose or gain and/or how much muscle the user would like to lose or gain. In another example, the request can include information regarding muscle toning. The request can also provide a timeline for when the use user would like to achieve the changes to the user's appearance.

In an aspect, in order to identify physical characteristics that the user would like to obtain using a health and fitness plan, the user can critique an image of himself or herself or avatar generated by visualization component 1318 that represents the user's appearance. For example, visualization component 1318 can generate an avatar that is presented to the user and represents the user's current appearance. Using the visualization, the user can point out various aspects of the user's physical features that the user would like to change and how the user would like to change them. For example, the user could indicate (e.g., by drawing on or marking up the avatar representation) where the user would like to gain muscle and how much muscle the user would like to gain. In another example, the user could mark places on his or her body whether he or she would like to trim fat or tone up.

In another aspect, in order to identify physical characteristics that the user would like to obtain using a health and fitness plan, the user provide an image of a person's body that the user's would like his or her body to resemble, or a particular body part that the user would like have his or her corresponding body part resemble. For example, in association with a request for a custom diet and fitness plan, the user can provide an image of himself or herself when they were in great shape years prior or a picture of a famous fitness model. In another example, the user could provide an image of a set of abdominals that the user would like his or her abdominals to look like.

In some respects, a user's aspirations may be unachievable or unhealthy. For example, a user may request he or she would like to appear a certain way within a period of two weeks and this request would require losing of an unhealthy amount of weight too quickly. Accordingly, program guide module 1320 can include risk component 1406 and regulation component 1408. Risk component 1406 is configured to evaluate a request and determine health risks associated with achieving the one or more desired changes to the user's physical appearance identified in the request based on the user's current appearance and health, profile information for the user and various existing health standards. In an aspect, when a request provides a duration of time over which the user would like to achieve the physical changes, risk component 1406 can also consider whether the user's aspirations are reasonable and/or achievable in a healthy manner within the duration of time.

Based on the health risks identified or determined by risk component 1406 regulation component 1408 is configured to determine whether the user's goal is obtainable in a healthy and safe manner (e.g., based on various health standards and risk allowance thresholds). For example, regulation component 1408 can return a response to a user's request indicating that the user's goal is or is not possible over the desired duration in a healthy manner and/or that the user's goal is not a recommended healthy goal (e.g., based on various existing health standards). For example, the user may desire to achieve a weight that is unhealthy for the user's height, age, and/or physical training routine (e.g., where the user is an athlete required to perform various physical activities). In an aspect, regulation component 1408 can automatically adjust aspects of a user's request to accommodate risks identified by risk component 1406. For example, regulation component 1408 can increase the duration of time of a health and fitness program to a duration that allows the user to achieve his or her goal in a reasonable manner. In another example, regulation component 1408 can slightly modify physical the degree of physical changes (e.g., the amount of weight lost/gained) request by the user to a reasonable value.

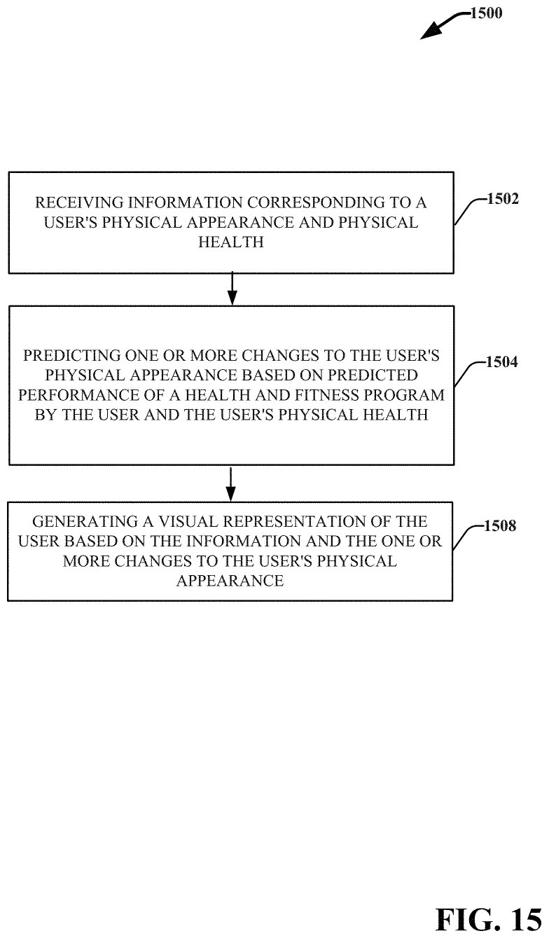

FIG. 15 illustrates a flow chart of an example method 1500 for generating a visualization representing how a user will appear in response to adhering to a health and fitness program, in accordance with various aspects and embodiments described herein. At 1502, information corresponding to a user's physical appearance and physical health is received (e.g., via reception component 1304). At 1504, one or more changes to the user's physical appearance are predicted based on predicted performance of a health and fitness program by the user and the user's physical health (e.g., via processing module 1306). At 1506, a visual representation of the user is generated based on the information and the one or more changes to the user's physical appearance (e.g., via visualization component 1318).

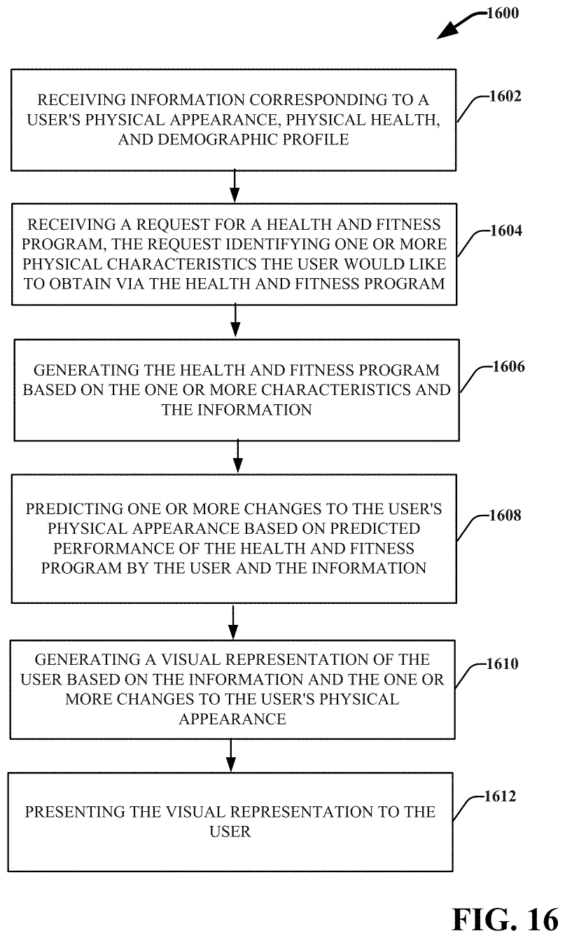

FIG. 16 illustrates a flow chart of another example method 1600 for generating a visualization representing how a user will appear in response to adhering to a health and fitness program, in accordance with various aspects and embodiments described herein. At 1602, information corresponding to a user's physical appearance, physical health and demographic profile is received (e.g., via reception component 1304). At 1604, a request is received for a health and fitness program, the request identifying one or more physical characteristics the user would like to obtain via the health and fitness program (e.g., via request component 1402). At 1606, the health and fitness program is generated based on the one or more characteristics and the information (e.g., via program guide module 1320). At 1608, one or more changes to the user's physical appearance are predicted based on predicted performance of a health and fitness program by the user and the user's physical health (e.g., via processing module 1306). At 1610, a visual representation of the user is generated based on the information and the one or more changes to the user's physical appearance (e.g., via visualization component 1318).

### V—Tracking and Rewarding Health and Fitness Activities Using Blockchain Technology

One or more embodiments described herein can include systems, computer-implemented methods, apparatus, and/or computer program products that can facilitate tracking and rewarding health and fitness activities of users using blockchain technology.

Blockchain technology is widely regarded as a revolutionary, peer-to-peer, decentralized option for data organization; it allows for formation of decentralized monetary systems such as crypto-coins, smart contracts, and other resources that can be managed online such as smart property. Blockchain can be used in distributed ledger systems and allows different entities to exchange data and transactions quickly without intervention or verification by third parties. This can be accomplished through a shared data framework that utilizes computer algorithms to create real-time self-updates. Blockchain technology can also settle financial transactions without mediation from banks and other trusted institutions.

A common framework for blockchain is a decentralized database in which transactions are recorded using a virtually unmodifiable cryptographic signature. Records can be added to the decentralized database to create blocks, that are protected against manipulation and alteration. Each block is connected to a previous block and has a timestamp of one or more transactions (e.g., the time at and/or about which such one or more transactions occurred), metadata characterizing the one or more transactions (e.g., amounts of cryptocurrencies involved in such one or more transactions, blockchain addresses from which and/or to which such amounts of cryptocurrencies were transferred during such one or more transactions), and/or a cryptographic hash of the previous electronic block. Because each electronic block can include a cryptographic hash of the previous electronic block, the blockchain can be resistant to retroactive tampering.

With this framework in mind, one or more embodiments described herein can employ a blockchain network to record and track health and fitness information for users. In various embodiments, the health and fitness information can include information regarding performance of fitness activities (e.g., performance of workouts, performance of specific exercises or physical moves, etc.) by users that can be received from a verified source. For example, in accordance with one or more of the avatar systems described above (e.g., system 100, system 200, system 1300 and/or system 1400), users can perform fitness activities (e.g., workout routines) in association with being monitored via an avatar guidance platform that can track user performance of fitness activities while providing guidance and feedback regarding the user's physical and physiological performance based on analysis of sensory feedback captured for the user (e.g., captured via one or more sensors 104 worn by the user and/or integrated withing an IDF 118) and/or image data captured of the user. In this regard, the avatar system can receive and/or determine and track information regarding completion of fitness activities, type of fitness activities completed, and duration of time of a workout or activity. The avatar system can also receive and/or determine and track performance metrics regarding physiological parameters of the user during the workout (e.g., heart rate, body temperature, respiration, perspiration, blood pressure, calories burned, etc.) and relative to individual exercise moves. The avatar system can also receive and/or determine and track information regarding specific exercise moves performed, number of reps, proper and improper form and motion, level of exertion, amount of weight used, information regarding physical fitness practices and abilities of the user (e.g., how fast the user can run, how high the user can jump, how much weight the user can lift, flexibility of the user, range of motion of the user, etc.).

The health and fitness information can also include information regarding physical measurements of a user (e.g., height, weight, BMI, specific body part measurements (e.g., waist, chest, upper arm/bicep, thigh, etc.), body fat, body composition, muscle composition, muscle density, etc.) generated in an automated manner via one or more via one or more devices. For example, as described with reference to system 100 (and additional systems described herein), in some embodiments, this information can be captured and reported to the client device 106 and/or the avatar server 116 via one or more sensors 104 and/or one or more IFDs 118 (e.g., smart scales, smart mirrors). Additionally, or alternatively, physical measurement data can be determined by the client device 106 and/or the avatar server 116 in an automated manner based on analysis of image data captured of the user. For example, image data for a user captured by visual capture device 110 can be analyzed using two-dimensional and/or three-dimensional image analysis to determine physical measurements metrics for the users, such as height, weight, body fat, dimension of the user's waist, hips, shoulders, biceps, etc., as a user's body changes over time (e.g., in response to performance of an exercise or diet program).

Because the avatar system receives and/or generates the health and fitness information based on directly monitoring the user during a workout via captured sensory feedback and/or image data captured of the user (e.g., via one or more sensors 104, one or more IFDs 118, and/or client device 106), the health and fitness information can be considered accurate and verified. In accordance with the disclosed embodiments, some or all of this health and fitness information received, captured and/or generated by the avatar system (as well as other verifiable health and fitness systems and devices) can be recorded for users on a blockchain overtime, thereby creating an immutable, verified record of each users' health and fitness activities and associated performance metrics over time.

In some embodiments, the blockchain can comprise a private (or semi-private) blockchain for the avatar system used to record and track health and fitness information for users thereof. In other embodiments, the blockchain can comprise a public or (or semi-public) blockchain shared by a plurality of different authorized entities (e.g., the avatar guidance system and other third-party systems/devices) to track and record health and fitness information for users. In accordance with these embodiments, information received for a user from different sources can be linked to the same user identity on the blockchain. In this regard, the blockchain can correspond to a shared, distributed ledger (e.g., shared between a plurality of different authorized systems/devices) that aggregates health and fitness information for user identities received from different entities authorized to write (e.g., record, encode, populate, etc.) to the blockchain and read from the blockchain (e.g., access and view the information recorded on the blockchain).

The different authorized entities can include a variety of entities in the health and fitness industry authorized that can generate and provide same or similar health and fitness information for users as the avatar systems described above (and/or other types of health and fitness information) in an automated and/or trusted and verifiable manner. In this regard, the different authorized entities can include other fitness service providers that provide fitness classes and/or coaching services to users in a manner that employs technology (e.g., wearables, sensors, smart-equipment, image/video data, etc.,) to track and verify performance of fitness activities by users. One example of such an entity includes Peloton®, an exercise equipment and media company that employs Internet-connected stationary bicycles and treadmills to enable monthly subscribers to remotely participate in fitness classes via streaming media. Another example of such an entity includes Mirror®, an Internet connected fitness system that streams live and on-demand classes to users in-home through a sleek responsive display. Other suitable entities can include health and fitness monitoring device providers and/or smart-equipment providers that can receive and track information regarding health and fitness activities performed by users via their proprietary devices and/or equipment. Other suitable entities include health and fitness tracking application providers that employ proprietary and non-proprietary wearable devices to track and record fitness activities for users, such as step-counting applications, running/walking/biking tracking applications, and the like.

The different authorized entities can also include online fitness coaching, training and/or physical therapy systems that provides remote fitness coaching, training and/or physical therapy services to users in real-time via real people (e.g., live tracking/interaction) that can track and verify performance of fitness activities by users.

The different authorized entities can also include brick-and-mortar health and fitness providers that provide health and fitness services to users at physical locations, such as gyms, physical therapy clinics, and fitness studios of a wide variety of different group fitness and/or private fitness activities (e.g., yoga, dance, martial arts, boxing, strength training, cross-fit, bootcamp, cycling, rowing, running, sports, etc.). Such entities can track and report attendance and completion of respective fitness activities using electronic attendance tracking systems in an automated matter (e.g., based on detection of user attendance as a function of location tracking, checking-in/checking-out via an automated system that tracks user identity verified presence via scanning an identity-linked card/device and/or facial recognition, etc.,) and/or in a manually verified manner. Some of these brick-and-mortar health and fitness systems may also employ wearables and/or smart equipment that can also track and generate additional performance metrics regarding specific exercises performed, equipment used, equipment settings (e.g., regarding weight, speed, duration, intensity, etc.) of individual exercises and workouts, calories burned, exertion levels, etc.), which can also be electronically recorded and reported in a verified manner.

In various embodiments, the blockchain can be made accessible to various authorized entities that can utilize the verified tracked health and fitness information for different individuals. For example, the blockchain can be made accessible to insurance provider systems to tailor insurance premiums and rates for the users based on their tracked health and fitness data (e.g., rewarding users with discounts and/or lower rates based on satisfaction of definable criteria). In another example, the blockchain can be made accessible to machine learning developer systems that can utilize the verified tracked health and fitness information for different users in association with machine learning model development/training and related applications.

Additionally, or alternatively, health and fitness information for users recorded on the blockchain can be used to provide them with rewards based on a satisfaction of variety of definable achievement criteria (e.g., completing N number of workouts, attaining a threshold performance level, performing N number of reps correctly, burning X number of calories, gaining N pounds of muscle, losing M pounds of weight, etc.). The rewards can be provided by various entities, including the avatar system, other users, third-party systems/devices authorized to write to the blockchain, third-party systems/devices authorized to read from the blockchain, and/or other third-party systems/devices that are unauthorized to read from and/or write to the blockchain.

In various embodiments, the rewards can be provided in the form of non-fungible (NFTs) tokens that can represent a variety of digital and real-world assets. For example, the NFTs can represent a digital collectible (e.g., digital artwork), a digital badge (e.g., an achievement badge), a digital coupon (e.g., redeemable for digital and/or real-world items), and/or a digital avatar item or accessory. In another example, the NFTs can represent a digital access pass (e.g., an entry/admission ticket or the like) to a real-world event or environment (e.g., concert tickets, sporting event tickets, theme park entry tickets, etc.). In another example, the NFTs can corresponds to a digital access pass to a VR environment (e.g., a VR game and/or world/room within the game), an AR reality environment (e.g., an AR game and/or world/room within the game), or an extended reality (XR) environment. The possibilities are endless.

In this regard, NFTs are cryptographic assets on blockchain with unique identification codes and metadata that distinguish them from each other. NFTs can be used to represent goods, financial assets, securities, and services (e.g., a token stored in a digital wallet can represent tickets to an event or for travel, artwork, most anything with digital value associated therewith). Unlike cryptocurrencies, they cannot be traded or exchanged at equivalency. This differs from fungible tokens like cryptocurrencies, which are identical to each other and, therefore, can be used as a medium for commercial transactions. NFTs can be used to represent real-world items like artwork and real-estate. NFTs can also be used to represent peoples' identities, property rights, and more.

Like physical money, cryptocurrencies are fungible e.g., they can be traded or exchanged, one for another. For example, one Bitcoin is always equal in value to another Bitcoin. This fungibility characteristic makes cryptocurrencies suitable for use as a secure medium of transaction in the digital economy. NFTs shift the crypto paradigm by making each token unique and irreplaceable, thereby making it impossible for one non-fungible token to be equal to another. They are digital representations of assets and have been likened to digital passports because each token contains a unique, non-transferable identity to distinguish it from other tokens. They are also extensible, meaning you can combine one NFT with another to “breed” a third, unique NFT. Non-fungible tokens are also excellent for identity management. Consider the case of physical passports that need to be produced at every entry and exit point. By converting individual passports into NFTs, each with its own unique identifying characteristics, it is possible to streamline the entry and exit processes for jurisdictions. Expanding this use case, NFTs can be used for identity management within the digital realm as well.

NFTs can also democratize investing by fractionalizing physical assets like real estate. It is much easier to divide a digital real estate asset among multiple owners than a physical one. That tokenization ethic need not be constrained to real estate; it can be extended to other assets, such as artwork. Thus, a painting need not always have a single owner. Its digital equivalent can have multiple owners, each responsible for a fraction of the painting. Such arrangements could increase its worth and revenues. Likewise, NFTs can be utilized to share assets associated with a digital wallet.

In some embodiments, NFTs rewards for health and fitness activity accomplishments can be issued to users automatically in response to satisfaction of definable achievement criteria using smart contracts encoded on the blockchain or another blockchain. Smart contracts clarify and interpret relations regarding the blockchain and supply chain. Different entities can create smart contracts based on a blockchain, without intervention from other entities. The data contained in such a smart contract is difficult to alter, as blocks cannot be changed after being formed. Thus, a smart contract on a blockchain can be a set of actions implemented on contributing nodes, creating mutual agreement in results of such transactions without need for third-party mediation of information and money. Parties negotiate and agree on conditions and a smart contract is created, coded, and archived in a blockchain structure. Once the contract is created, and the conditions for an exchange are fulfilled, goods and funds (e.g., NFTs) are transferred according to terms of the contract upon satisfaction of the terms based on the verifiable data recorded on the blockchain (e.g., user health and fitness activity data as applied to various instant embodiments). No human mediation is required, and the transaction can proceed faster, and at less cost than a conventional paper-centric contract process. As most if not all participants in a network, associated with the transaction, have a copy of every transaction record, there is improved mutual trust.

In accordance with various embodiments, smart contracts that control provision of NFT rewards to users based health and fitness achievement criteria can be created by various entities. For example, the smart contracts can be created by the avatar system, other third-party health and fitness systems that record user health and fitness activity data on the blockchain, and other third-party retailors that provide goods and services that can be represented and gifted as NFTs (e.g., health and fitness merchants and other merchants). The smart contracts can also be created by other users. For example, a friend, family member, personal trainer, etc., of one user may create a smart contract for the user that effectuates provision of an NFT reward (or another digital asset reward such as cryptocurrency) to the user in response to satisfaction of some definable health and fitness criteria that can be tracked and verified on the blockchain (e.g., completing X number of workouts, losing N number of pounds, etc.).

In an embodiment, a system, comprises a non-transitory memory comprising instructions; and one or more hardware processors coupled to the non-transitory memory and configured to read the instructions to cause the system to perform operations. The operations can comprise receiving health and fitness information for entities (e.g., users and/or groups of users). For example, the health and fitness information can relate to performance of one or more fitness activities (e.g., activity type, duration, number of completions, etc.), physical fitness performance metrics (e.g., measures of intensity and/or duration, calories burned, heart-rate, level of exertion, etc.), body measurements (e.g., weight gain/loss, muscle gain/loss, BMI, etc.). The operations further comprise, employing a blockchain network to store the health and fitness information for the entities, and sending a NFT token to an entity of the entities in response to a subset of the health and fitness information associated with the entity satisfying a criterion (e.g., a definable criterion as a function of a measure of achievement with respect to the activities performed). In some aspects, the sending comprises sending the non-fungible token to an address on the blockchain network associated with the entity. In some aspects, the sending is responsive to self-execution of a smart contract as a function of the subset of the health and fitness information becoming stored on the blockchain network, and wherein the smart contract is encoded on the blockchain network.

The NFT can correspond to various digital and real-world items. In some implementations, The NFT corresponds to an item selected from the group consisting of: a digital collectible, a digital badge, a digital coupon and a digital avatar item or accessory. In another example, the NFTs corresponds to a digital access pass to a real-world event or environment. In another example, the NFT corresponds to a digital access pass to a virtual reality environment, an augmented reality environment, or a mixed reality environment.

In various implementations, the receiving comprises receiving the health and fitness information from one or more authorized data sources (e.g., the avatar system, sensors 104, an IFD 118, and other authorized devices/systems), and wherein the employing comprises storing the health and fitness information on the blockchain network in response to the receiving. The operations can further comprise linking respective portions of the health and fitness information corresponding to respective entities of the entities with respective addresses of the entities of the blockchain network

Referring now to the drawings, FIG. 17 illustrates a high-level block diagram of an example, non-limiting system 1700 that can facilitate tracking and rewarding health and fitness activities of users using blockchain technology, in accordance with one or more embodiments described herein. System 1700 comprises some elements of the avatar systems described above, including user 102, one or more sensors 104, IFD 118, client device 106 and avatar server 116. These elements can provide any of the corresponding functionalities previously described with reference to FIGS. 1-16 and/or the additional or alternative functionalities described below. For example, in some embodiments, the client device 106 can include rendering component 108, visual capture device 110, sensor module 112, and/or avatar platform 114. Likewise, the avatar server 116 can include avatar platform 114, one or more components of avatar guidance platform 202, one or more components of avatar visualization platform 1302, and/or one or more components of avatar visualization platform 1302. Repetitive description of like elements employed in respective embodiments is omitted for sake of brevity.

System 1700 further includes blockchain network 1702, data storage 1714, and a plurality of other computing systems/devices, including health/fitness systems 1716, insurance provider systems 1718, retailer systems 1720, XR systems 1722, and other trusted sources/systems 1724. The avatar server 116, the client device, the IFD 118, the one or more sensors 104, the data storage 1714 and the various additional systems (e.g., health/fitness systems 1716, insurance provider systems 1718, retailer systems 1720, XR systems 1722, and other trusted sources/systems 1724) can be communicatively coupled to the blockchain network 1702 via any suitable wired and/or wireless communication technology.

In its broadest sense, blockchain refers to a framework that supports a trusted ledger that is stored, maintained, and updated in a distributed manner in a peer-to-peer network. For example, in a cryptocurrency application, such as Bitcoin or Ethereum, Ripple, Dash, Litecoin, Dogecoin, zCash, Tether, Bitcoin Cash, Cardano, Stellar, EOS, NEO, NEM, Bitshares, Decred, Augur, Komodo, PIVX, Waves, Steem, Monero, Golem, Stratis, Bytecoin, Ardor, or in digital currency exchanges, such as Coinbase, Kraken, CEX.IO, Shapeshift, Poloniex, Bitstamp, Coinmama, Bisq, LocalBitcoins, Gemini and others the distributed ledger represents each transaction where units of the cryptocurrency are transferred between entities. For example, using a digital currency exchange, a user may buy any value of digital currency or exchange any holdings in digital currencies into worldwide currency or other digital currencies. Each transaction can be verified by the distributed ledger and only verified transactions are added to the ledger. The ledger, along with many aspects of blockchain, may be referred to as “decentralized” in that a central authority is typically not present. Because of this, the accuracy and integrity of the ledger cannot be attacked at a single, central location. Modifying the ledger at all, or a majority of, locations where it is stored is made difficult so as to protect the integrity of the ledger. This is due in large part because individuals associated with the nodes that make up the peer-to-peer network have a vested interest in the accuracy of the ledger.

Though maintaining cryptocurrency transactions in the distributed ledger may be the most recognizable use of blockchain technology today, the ledger may be used in a variety of different fields. Indeed, blockchain technology is applicable to any application where data of any type may be accessed where the accuracy of the data is assured. For example, a supply chain may be maintained in a blockchain ledger, where the transfer of each component from party to party, and location to location, may be recorded in the ledger for later retrieval. Doing so allows for easier identification of a source for a defective part and where other such defective parts have been delivered. Similarly, food items may be tracked in like manner from farm to grocery store to purchaser.

In accordance with the embodiment shown, the blockchain network 1702 can comprise a blockchain 1704 and a plurality of nodes 1703₁₋ₖ. The number of nodes k can vary. These nodes 1703₁₋ₖ can correspond to any suitable computing device (e.g., real or virtual) that stores a copy or instance of the blockchain 1704 in a suitable memory and executes the corresponding blockchain technology (i.e., execution instructions 1712) defined for the blockchain 1704 using one or more processors. In this regard, the blockchain 1704 can corresponds to a trusted ledger that is stored, maintained, and updated in a distributed manner in a peer-to-peer network via the respective nodes 1703₁₋ₖ.

The blockchain 1704 can include a set of blockchain address 1706 that correspond to different entities/users, and more particularly to different digital accounts (e.g., similar to user accounts, bank accounts, or the like) of the different entities/users. In various embodiments, the blockchain addresses 1706 can be linked to corresponding digital wallets of the different entities/users (e.g., digital wallet 1728, digital wallet 1726 and the like). The blockchain 1704 can also include blockchain records 1708 which can correspond to any information recorded on the blockchain in accordance with the execution instructions 1712. For example, as applied to cryptocurrency applications, such as Bitcoin, the blockchain records 1708 may correspond to cryptocurrency transactions. As applied to various embodiments of the disclosed subject matter, the blockchain records 1708 can include health and fitness information tracked and recorded for different users/entities, NFTs, and other transactions (e.g., exchange of NFTs and other digital assets between users/entities).

In various instances, the blockchain records 1708 can be linked to corresponding blockchain addresses of respective entities/user for which the respective records pertain. For example, a blockchain record recording health and fitness information for a particular user/entity can be linked to their corresponding blockchain address. Accordingly, the blockchain 1704 can be considered as a digital record of the set of blockchain records 1708 for a set of blockchain addresses 1706 corresponding to different users/entities. In some embodiments, some or all of the tracked health and fitness information for can be stored in a data store 1714 that is external to the blockchain and the blockchain records 1708 can included hashed data entries that immutable link to the corresponding tracked health and fitness information as aggregated and stored externally. The blockchain 1704 can further include smart contracts 1710 and execution instructions 1712.

The blockchain 1704 can include any suitable blockchain capable of recording data (e.g., health and fitness data, NFTs and smart contracts) and executing smart contracts 1710 in a verifiable and immutable manner. In various embodiments, the blockchain 1704 can be a decentralized digital ledger made up of a sequence of electronic blocks. In various instances, any given electronic block can include a timestamp (e.g., time and/or date) associated with one or more blockchain records 1708 (e.g., some of the set of blockchain transactions 108). In various cases, the given electronic block can also include metadata pertaining to the one or more blockchain records, such as the source of the data (e.g., the particular sensor 104, IFD 118, client device 106, digital wallet, avatar server 116, health and fitness system 1716 and so on), the location of the data on external storage 1714 (in implementations in which some or all of the actual data is stored externally), a user identity associated with the data, and/or respective blockchain addresses associated with the data. In some cases, the given electronic block can further include metadata pertaining to the one or more blockchain addresses, such as age of a blockchain address (e.g., days, months, and/or years since registration and/or creation of the blockchain address) and/or number of smart contract tokens obtained by a blockchain address. Moreover, in various aspects, the given electronic block can include any suitable cryptographic hash of the preceding electronic block. In various instances, because each electronic block in the blockchain 1704 can include a cryptographic hash of the previous electronic block, the blockchain 1704 can be resistant to retroactive tampering and/or falsification.

In accordance with various embodiments, the blockchain records 1708 can comprise health and fitness information tracked for a plurality of users (e.g., user 102 and the like) wherein each user can be linked to their corresponding health and fitness information by one or more unique identifiers (e.g., names, device IDs, account numbers, profile names, etc.) and/or a corresponding blockchain address 1706. The health and fitness information can include information regarding performance of fitness activities (e.g., performance of workouts, performance of specific exercises or physical moves, etc.) by users that can be received from a verified source. For example, the health and fitness information can comprise information regarding completion of fitness activities, type of fitness activities completed, and duration of time of a workout or activity. The health and fitness information can also include performance metrics regarding physiological parameters of the user during the workout (e.g., heart rate, body temperature, respiration, perspiration, blood pressure, calories burned, etc.) and relative to individual exercise moves. The health and fitness information can also include performance metrics regarding specific exercise moves performed, number of reps, proper and improper form and motion, level of exertion, amount of weight used, and information regarding physical fitness practices and abilities of the user (e.g., how fast the user can run, how high the user can jump, how much weight the user can lift, flexibility of the user, range of motion of the user, etc.). The health and fitness information can also include information regarding physical measurements of a user (e.g., height, weight, BMI, specific body part measurements (e.g., waist, chest, upper arm/bicep, thigh, etc.), body fat, body composition, muscle composition, muscle density, etc.) generated in an automated manner via one or more via one or more devices.

In one or more embodiments, the execution instructions 1712 can control what entities (e.g., systems/devices) can write to the blockchain 1704 (in association with recording blockchain records 1708), what information can be recorded on the blockchain 1704 (e.g., what the blockchain records can contain), what entities can access and read the information on the blockchain 1704, and permissions regarding subsets of the information the entities have access to (e.g., not all entities may have access to view some personal/private information of users). In accordance with various embodiments in which the blockchain 1704 is employed to record health and fitness information for users (e.g., user 102 and the like), the execution instructions 1712 can define one or more verified entities that can record such information on the blockchain 1704 (e.g., as blockchain records). In this context, a verified entity refers to a system, device, user account, or the like from which information is received that is assumed to be accurate and true by the blockchain network 1702. For example, verified entities authorized to record health and fitness information on the blockchain can include specific sensors 104 that directly capture and report the data, specific IFDs 118 that directly capture and report the data, specific client devices 106 with hardware (e.g., cameras, health-tracking sensors/hardware, location tracking devices, etc.) and/or software (e.g., applications, programs) that directly capture, determine and/or report the data, the avatar server 116, and various other verified health and fitness systems 1716 that employ technology (e.g., sensors, IFDs, wearables, image capture devices) to generate and provide verified health and fitness data for users and/or that employ manually verified data reporting (e.g., an authorized person verifying a user performed a specific fitness activity or achieved a specific health/fitness performance metric). Accordingly, users cannot self-report and record their own health and fitness information on the blockchain 1704 to prevent the scenario in which a user may inaccurately report they have performed a workout, lost X amount of weight or the like, when in fact they have not.

In this regard, the source of the health and fitness information can vary so long as the blockchain network 1702 considers the source verified. In some embodiments, the blockchain network 1702 can comprise a private (or semi-private) blockchain (e.g., blockchain 1704 can be a private blockchain) for the avatar server 116 used to record and track health and fitness information for only users thereof. With these embodiments, the avatar server 116 can be configured to receive and/or determine the health and fitness information for the respective users based on their activity/usage of the avatar system using the techniques described above. The avatar server 116 can further be configured to submit and record the respective users' health and fitness information in response to reception thereof and/or generation thereof. Additionally, or alternatively, one or more devices employed by the avatar system (e.g., sensors 104, IFD 118, client device 106) can be configured to automatically send the corresponding health and fitness information captured and/or determined thereby directly to the blockchain network 1702, wherein based on sending and confirming the source is verified (e.g., as defined by the execution instructions 1712), the blockchain network encodes/records the data on the blockchain 1704.

In other embodiments, the blockchain 1704 can comprise a public or (or semi-public) blockchain shared by a plurality of different authorized and verified entities (e.g., the avatar system and other third-party systems/devices) to track and record health and fitness information for users. Information received for a user from different sources can be linked to the same user identity and/or blockchain address for the user identity on the blockchain 1704. With these embodiments, the blockchain 1704 can correspond to a shared, distributed ledger (e.g., shared between a plurality of different authorized systems/devices) that aggregates health and fitness information for user identities received from different entities authorized to write (e.g., record, encode, populate, etc.) to the blockchain and read from the blockchain (e.g., access and view the information recorded on the blockchain). In the embodiment shown, these different authorized entities are represented as health and fitness systems 1716.

The health and fitness systems 1716 can include a variety of entities in the health and fitness industry authorized that can generate and provide same or similar health and fitness information for users as the avatar server 116 (and/or devices coupled to the avatar server 116) described above (and/or other types of health and fitness information) in an automated and/or trusted and verifiable manner. In this regard, the health and fitness systems 1716 can include other fitness service providers that provide fitness classes and/or coaching services to users in a manner that employs technology (e.g., wearables, sensors, smart-equipment, image/video data, etc.,) to track and verify performance of fitness activities by users (e.g., Peloton®, Mirror®, and the like). The health and fitness systems 1716 can also include health and fitness monitoring device providers and/or smart-equipment providers that can receive and track information regarding health and fitness activities performed by users via their proprietary devices and/or equipment. The health and fitness systems 1716 can also include health and fitness tracking application providers that employ proprietary and non-proprietary wearable devices to track and record fitness activities for users, such as step-counting applications, running/walking/biking tracking applications, and the like. The health and fitness systems 1716 can also include online fitness coaching, training and/or physical therapy systems that provides remote fitness coaching, training and/or physical therapy services to users in real-time via real people (e.g., live tracking/interaction) that can track and verify performance of fitness activities by users. The health and fitness systems 1716 an also include brick-and-mortar health and fitness providers that provide health and fitness services to users at physical locations, such as gyms, physical therapy clinics, and fitness studios of a wide variety of different group fitness and/or private fitness activities (e.g., yoga, dance, martial arts, boxing, strength training, cross-fit, bootcamp, cycling, rowing, running, sports, etc.). Such entities can track and report attendance and completion of respective fitness activities using electronic attendance tracking systems in an automated matter (e.g., based on detection of user attendance as a function of location tracking, checking-in/checking-out via an automated system that tracks user identity verified presence via scanning an identity-linked card/device and/or facial recognition, etc.,) and/or in a manually verified manner. Some of these brick-and-mortar health and fitness systems may also employ wearables and/or smart equipment that can also track and generate additional performance metrics regarding specific exercises performed, equipment used, equipment settings (e.g., regarding weight, speed, duration, intensity, etc.) of individual exercises and workouts, calories burned, exertion levels, etc.), which can also be electronically recorded and reported in a verified manner.

In various embodiments, the blockchain 1704 can be made accessible to various authorized entities that can utilize the verified tracked health and fitness information for different individuals. For example, the blockchain can be made accessible to insurance provider systems 1718 to tailor insurance premiums and rates for the users based on their tracked health and fitness data (e.g., rewarding users with discounts and/or lower rates based on satisfaction of definable criteria). In another example, the blockchain can be made accessible to machine learning developer systems (e.g., one or more of the other trusted sources/systems 1724) that can utilize the verified tracked health and fitness information for different users in association with machine learning model development/training and related applications.

Additionally, or alternatively, health and fitness information for users recorded on the blockchain 1704 can be used to provide them with rewards based on a satisfaction of variety of definable achievement criteria (e.g., completing N number of workouts, attaining a threshold performance level, performing N number of reps correctly, burning X number of calories, gaining N pounds of muscle, losing M pounds of weight, etc.). The rewards can be provided by various entities, including the avatar server 116, other users, third-party systems/devices authorized to write to and/or read from the blockchain 1704, (e.g., retailer systems 1720, XR systems 1722), and/or other third-party systems/devices that are unauthorized to read from and/or write to the blockchain.

In various embodiments, the rewards can be provided in the form NFTs that can represent a variety of digital and real-world assets. For example, the NFTs can represent a digital collectible (e.g., digital artwork), a digital badge (e.g., an achievement badge provided by the avatar server 116 and/or one or more health and fitness systems 1716), a digital coupon (e.g., provided by one or more retailor systems 1720 redeemable for digital and/or real-world items), and/or a digital avatar item or accessory. In another example, the NFTs can represent a digital access pass (e.g., an entry/admission ticket or the like) to a real-world event or environment (e.g., concert tickets, sporting event tickets, theme park entry tickets, etc., provided by one or more retailer systems 1720). In another example, the NFTs can corresponds to a digital access pass to an XR environment (e.g., a VR, AR or XR game and/or world/room within the game) provided by one or more XR systems 1722. The possibilities are endless.

In accordance with these embodiments, the NFT rewards can be recorded and stored on the blockchain 1704. In this regard, the blockchain records 1708 can comprise information identifying the owner (i.e., user, system, company, etc.) of the NFT, the asset (e.g., digital or real) that the NFT represents, and linking the NFT to the corresponding blockchain address (of blockchain addresses 1706) associated with the current owner. In some implementations, the NFT rewards can be existing on the blockchain and associated with the reward provider (i.e., the entity providing the reward) prior to provision of the NFT to a particular user as a reward. With these embodiments, the reward provider can transfer the NFT reward (i.e., transfer ownership of the NFT) to the corresponding user when earned (e.g., in response to satisfaction of the requisite health and fitness achievement criteria). Transferring or providing an NFT to a particular user refers to recording ownership of the NFT with the corresponding blockchain address of the new owner or sending the NFT to the blockchain address of the new owner. In embodiments, the NFT provider and/or the blockchain network 1702 (in accordance with defined NFT minting rules provided in the execution instructions 1712) can mint the NFT in the recipient's name in response to earning it (e.g., in response to satisfaction of the requisite health and fitness achievement criteria). Minting refers to the process of converting creating an NFT that represents a digital or real-world asset and recording the NFT on the blockchain 1704.

In various embodiments, reward NFTs (and any other type of NFT described herein) can be linked to respective entities to which they are owned (e.g., reward providers prior to distribution, users to which they are provided as rewards, and the like) via digital wallets (e.g., digital wallet 1726, digital wallet 1728, and the like). A digital wallet is a software component stored a device associated with the digital wallet owner (e.g., the user's client device 106 such as their smartphone, the avatar system server 116, or another device), or a hard device kept by the owner (e.g., a USB type device) that allows users to interact with blockchain networks. They are necessary when sending and receiving bitcoin and other digital currencies. The digital wallet generates a private key to the blockchain address of the asset on the blockchain. In this regard, users can access their NFTs as associated with their corresponding blockchain address on the blockchain 1704 using their digital wallet (e.g., digital wallet 1728) and the corresponding private key. The private key can also be required to authorize any transaction on that address. In this regard, unlike normal wallets that hold your credit cards and cash, digital wallets don't really store your funds. Coins or tokens (i.e., NFTs) are part of a blockchain network 1702 as data, and the digital wallet acts as a means to access them—whether it be sending them to another entity or retrieving them for usage thereof. Instead, the digital wallets (e.g., digital wallet 1728, digital wallet 1726) provides access to the assets, which are held on the blockchain 1704. It does so by providing a private key to a defined blockchain address where your assets are stored, which allows the wallet owner to authorize transactions. If you have the private key, you effectively own anything at that address. Users can also have their driver's license, health card, loyalty card(s) and other ID documents stored within their digital wallet and/or stored on their blockchain address and linked to their digital wallet. The credentials can be passed to a merchant's terminal wirelessly via near field communication (NFC).

There are two main types of digital wallets: hardware and software (also known as cold and hot storage wallets, respectively). Hot storage wallets are accessible via an internet service, e.g., Coinbase®, UpHold®, Voyager®, Binance® . . . , and these can be further segregated into online wallets and client-side wallets (e.g., managed locally on a user's computer or mobile device, such as client device 106). Although innovations described herein are mainly described within the context of client-side, hardware-based wallets, it is to be appreciated that the subject innovations are not intended to be limited to such implementation. Accordingly, in addition to client-side, hosted, proxied, distributed, hybrid or cloud-based implementations/embodiments of the device are contemplated. In various embodiments, all parties associated with NFT transactions on the blockchain network 1702 (e.g., users and NFT reward providers) can be associated with their own digital wallets 1728 and blockchain addresses.

In some embodiments, NFTs rewards for health and fitness activity accomplishments can be issued to users automatically in response to satisfaction of definable achievement criteria using smart contracts 1710 encoded on the blockchain 1704 (or another blockchain). Smart contracts clarify and interpret relations regarding the blockchain and supply chain. Different entities can create smart contracts based on a blockchain, without intervention from other entities. The data contained in such a smart contract is difficult to alter, as blocks cannot be changed after being formed. Thus, a smart contract on the blockchain 1704 can be a set of actions implemented on contributing nodes (e.g., nodes 1-6), creating mutual agreement in results of such transactions without need for third-party mediation of information and money. Parties negotiate and agree on conditions and a smart contract is created, coded, and archived in a blockchain structure. Once the contract is created, and the conditions for an exchange are fulfilled, goods and funds (e.g., NFTs) are transferred according to terms of the contract upon satisfaction of the terms based on the verifiable data recorded on the blockchain (e.g., user health and fitness activity data as applied to various instant embodiments). For example, the smart contracts 1710 can include a contract created by the avatar server 116 that controls provision of a particular NFT reward (e.g., a digital badge, a digital coupon exchangeable for real goods and/or services, etc.) to any user that completes N workouts, runs X miles, and so on. Once information has been recorded for a user on the blockchain 1704 that affirms the user has completed N workouts (e.g., as determined via the respective nodes 1-6), the smart contract will self-execute and result in automatically providing the user with the corresponding NFT reward. For example, the user can receive a notification (e.g., generated by the blockchain network 1702 and/or the reward provider) that they have received an NFT reward, information describing the reward and so on, an access their reward at their corresponding blockchain address using their private key and digital wallet.

In accordance with various embodiments, smart contracts 1710 that control provision of NFT rewards to users based health and fitness achievement criteria can be created by various entities. For example, the smart contracts can be created by the avatar server 116, other third-party health and fitness systems that record user health and fitness activity data on the blockchain (e.g., health/fitness systems 1716), and other third-party retailors that provide goods and services that can be represented and gifted as NFTs (e.g., health and fitness merchants and other merchants, such as retail systems 1720, XR systems 1722, and so on). The smart contracts can also be created by other users. For example, a friend, family member, personal trainer, etc., of one user may create a smart contract for the user that effectuates provision of an NFT reward (or another digital asset reward such as cryptocurrency) to the user in response to satisfaction of some definable health and fitness criteria that can be tracked and verified on the blockchain (e.g., completing X number of workouts, losing N number of pounds, etc.).

Embodiments of systems and devices described herein can include one or more machine-executable components or instructions embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described. These computer/machine executable components or instructions (and other described herein) can be stored in memory associated with the one or more machines. The memory can further be operatively coupled to at least one processor, such that the components can be executed by the at least one processor to perform the operations described. Examples of said and memory and processor as well as other suitable computer or computing-based elements, can be found with reference to FIG. 35 (e.g., processing unit 3504 and system memory 3506 respectively), and can be used in connection with implementing one or more of the systems or components shown and described in connection with FIG. 17, or other figures disclosed herein.

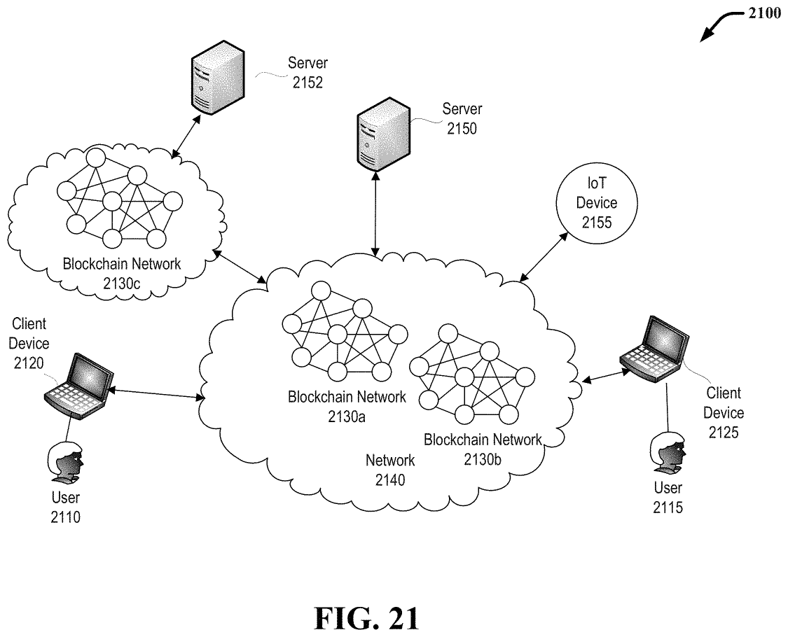

In this regard, it should be appreciated that the blockchain network 1702 comprises an interconnected network of devices. These devices can include the nodes 1703₁₋ₖ, as well as one or more servers and can respectively include or be operatively coupled to memory that stores the blockchain 1704 and a processor that executes the computer executable instructions and/or components associated therewith (e.g., the smart contracts 1710, the execution instructions 1712, and the like). Additional examples of the blockchain network 1702, the blockchain 1704, and the computer executable instructions and corresponding operations associated therewith are described with reference to FIGS. 21-27. It should also be appreciated that the various other devices and system of system 1700 (e.g., the client device 106, the avatar server 116, the IFD 118 in some implementations, the health and fitness systems 1716, the insurance provider systems 1718, the retailer systems 1720, the XR systems 1722, and the other trusted sources/systems 1724) can also can respectively include or be operatively coupled to memory that stores machine/computer executable instructions and a processor that executes the computer executable instructions and/or components associated therewith (e.g., the smart contracts 1710, the execution instructions 1712, and the like).

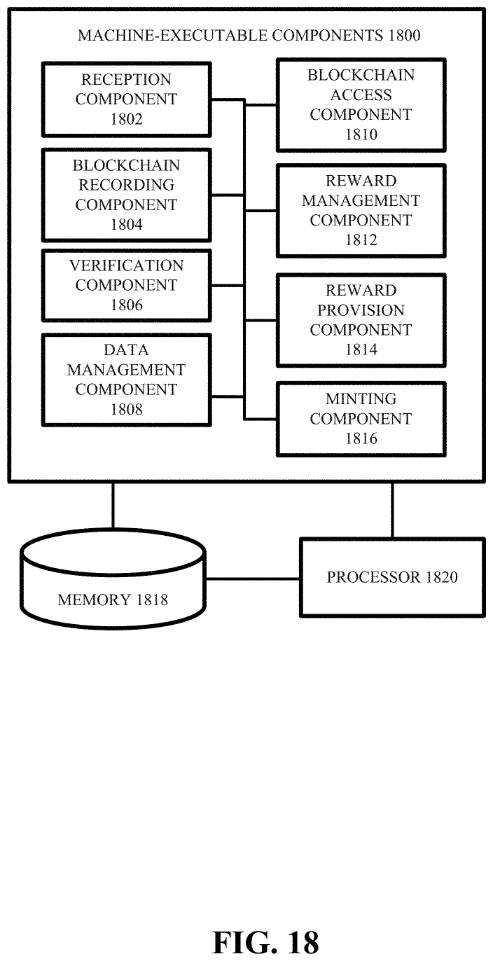

FIG. 18 illustrates example machine-executable components 1800 that can facilitate tracking and rewarding health and fitness activities of users using blockchain technology, in accordance with one or more embodiments described herein. The machine-executable components 1800 include reception component 1802, blockchain recording component 1804, verification component 1806, data management component 1808, blockchain access component 1810, reward management component 1812, reward provision component 1814, and minting component 1816. The machine-executable components 1800 can be stored in at least one computer-readable memory 1818 that is operably and/or operatively and/or communicatively connected/coupled to at least one processor 1820 (e.g., a computer processing unit, a microprocessor, etc.), upon execution by the processor 1820, can cause the processor 1820 to perform the corresponding acts or operations of the components described below. In some embodiments, the processor 1820, the memory 1818 and the machine-executable components 1800 can be associated with one or more devices of the blockchain network 1702. Additionally, or alternatively, the processor 1820, the memory 1818 and the machine-executable components 1800 (and/or one or more of the machine-executable components 1800) can be associated with the client device 106, the avatar server 116, the IFD 118 in some implementations, the health and fitness systems 1716, insurance provider systems 1718, the retailer systems 1720, the XR systems 1722, and the other trusted sources/systems 1724. Various different implementations and architectural configurations are envisioned.

With reference to FIG. 18 in view of FIG. 17, the reception component 1802 can receive health and fitness information for users to be recorded on the blockchain 1704. As described above with reference to system 1700, the health and fitness information can be received from one or more sensors 104, one or more IFDs 118, one or more client device 106, the avatar server 116, and/or one or more health and fitness systems 1716. The blockchain recording component 1804 can record the health and fitness information received for respective users on the blockchain 1704 (e.g., as blockchain records 1708) in response to reception therefor and/or in response to verification (e.g., by the verification component 1806) that the health and fitness information satisfies one or more defined rules and/or requirements for recording on the blockchain 1704 (e.g., defined in the execution instructions 1712). In various embodiments, these rules and requirements can define verified source from which the health and fitness information can be received (e.g., verified devices and/or systems) and/or are otherwise permissioned to write to the blockchain 1704. The verified sources can be identified by unique identifiers and/or unique public and/or private keys. The rules and requirements for recording on the blockchain can also control the specific type of information to be recorded, the format for recording, and the protocol for recording the data (e.g., the hashing and encryption protocol). In some embodiments, the recording protocol and involve recording an entry for a received health and fitness event for a user with a link to the actual information as recorded and stored at an external database (e.g., data storage 1714). In some embodiments, the data management component 1808 can control where specific types of received health and fitness information is stored and recorded (e.g., on the blockchain 1704 and/or at the external data storage 1714).

The data management component 1808 can also manage aggregating respective portions of the health and fitness information received for same users and associating that information with a distinct blockchain address (e.g., of blockchain addresses) designated for the respective users. In this regard, in association with recording health and fitness information received for different users on the blockchain 1704 (e.g., as blockchain records 1708), the blockchain recording component 1804 can link information received for a particular user with their corresponding blockchain address. The verification component 1806 and/or the data management component 1808 can employ various techniques to identify and verify the specific user to which a particular received data object or set of data is associated with. For example, in some embodiments, received health and fitness information can include one or more user identifiers that uniquely identify the user/entity to which it pertains. In some embodiments in which the health and fitness information can be received from a plurality of different systems/devices and/or applications in which the user has different accounts with the respective systems/device and/or applications, information linking the different user accounts (and corresponding usernames/identifier, such as usernames) for the respective disparate systems to a singular user identity and blockchain address can be stored on the blockchain and/or otherwise made accessible to the verification component 1806 and/or the data management component 1808.

The blockchain access component 1810 can control permissions regarding what entities (e.g., users, systems, devices, applications, etc.) can read from the blockchain 1704 and what information they can read (e.g., which may include all or portions of the information). The blockchain access component 1810 can also control permissions regarding what entities can submit and record smart contracts on the blockchain 1704 and what information can be defined in the smart contracts.

The reward management component 1812 can control and manage rules and protocols regarding providing users with rewards based on their respective health and fitness information. The reward provision component 1814 can provide users with rewards in response to satisfaction of defined health and/or fitness criterion (e.g., completing X number of workouts, losing N number of pounds, running X number of miles, burning N number of calories, performing N number of reps correctly, etc.). In various embodiments, this can involve providing a user with a digital asset such as a reward NFT or an amount of cryptocurrency. The reward NFT can represent essentially any real-world or digital object/file (e.g., a digital badge, certificate, image, video, song, or another type of digital collectable), a coupon redeemable for real-world objects or merchandise and/or services, concert tickets, theme park tickets, access passes to physical locations and virtual and/or other XR environments, avatar clothing, avatar accessories, avatar permissions (e.g., for games, environments, rooms within an environment, etc.). In accordance with these embodiments, based on a determination that a users' health and fitness information satisfies the defined criteria for receiving a reward NFT or another type of digital asset, the reward provision component 1814 can execute a blockchain transaction that involves sending and recording the NFT or digital asset to the blockchain address associated with that user. The reward provision component 1814 can further provide the user with a notification (e.g., to a device associated with the user and the user's blockchain address) indicating the user has received a reward. The user can further access and retrieve the reward using their digital wallet (e.g., digital wallet 1728) as linked to their blockchain address in association with providing the requisite security verification (e.g., a private key and/or digital signature).

As described above, in some embodiments, the rewards can be provisioned automatically using smart contracts recorded on the blockchain 1704. A smart contract is an agreement that is stored in a blockchain 1704 (e.g., as a blockchain record 1704) and automatically executed (or self-executed) when the agreement's predetermined terms and conditions are met. The terms and conditions of the agreement may be visible to other users of the blockchain. When the pre-defined rules are satisfied, then the relevant code is automatically executed and the reward defined by the smart contract is automatically provisioned (e.g., by the reward provision component 1814). The agreement may be written as a script using a programming language such as Java, C++, JavaScript, VBScript, PHP, Perl, Python, Ruby, ASP, Tcl, etc. The script may be uploaded to the blockchain as a transaction on the blockchain. The reward management component 1812 can facilitate defining and recording smart contracts on the blockchain 1704 for the provision of rewards to users based on variety of definable health and fitness achievement criteria. The reward management component 1812 can also manage protocols for screening and parsing the information recoded on the blockchain 1704 in association with determining when the rules and conditions of a smart contract have been met (or not), using a consensus model or another suitable model.

The rewards can be provided by essentially any authorized entity, including other users, the avatar server 116, the health and fitness systems 1716, retailor systems 1720, XR systems 1722 in accordance with defined rules and regulations for provision of rewards managed by the reward management component 1812 (and stored in the execution instructions). The criteria (e.g., rules and conditions) of the smart contracts can vary, be user definable and encompass essentially any verifiable aspect of the recorded health and fitness information. For example, the rules and conditions can relate to a specific type of workout or exercise and/or a measure of quantity, frequency, duration, distance, intensity, correctness of form, or another measurable performance metric. The rules and conditions can also include time and/or location restrictions. The rules and conditions can also relate to competing or relative fitness activities of other users (e.g., the top three fitness performs of a particular activity receive a reward).

As an example, a smart contract may be utilized between a first user and a second user for the provision of a reward to the first user based on the first user satisfying some defined health and/or fitness achievement, such as performing at least five hours of fitness activity a week for four weeks. The smart contract may indicate that the second user (e.g., a friend or family member, a merchant, a service provider, etc.) agrees to provide the first user with a reward (e.g., a digital asset such as a reward NFT or cryptocurrency) if the first user achieves the fitness criteria. In another example, a smart contract can be utilized by a fitness service provider to automatically provide users with achievement points or achievement badges for attaining definable fitness achievements during a workout (e.g., based on completing each exercise, based on completing certain exercises correctly, based on completing certain exercises with a threshold intensity level, etc.). In another example, the terms of a smart contract can include providing runners in a race with rewards in the form of points, badges, coupons, etc., each time the runners pass certain locations and/or mile markers. The terms of a smart contract can also include a gamification aspect in which rewards are provisioned based on relative fitness activity of two or more users. For example, in furtherance to the racing example, a smart contract can define terms that provide the first X number of runners to pass a certain mile marker with a reward NFT (e.g., some type of valuable prize or coupon).

In embodiments in which the smart contract controls provisioning of an NFT reward to one or more users based on definable health and/or fitness criteria the NFT can be either previously existing and transferred to the respective users based on satisfaction of the criteria or minted and provisioned to the respective users based on satisfaction of the criteria. In this regard, in embodiments in which the reward is transferred, the provisioning of the reward can correspond to the execution of a transaction in which the reward NFT is transferred from a first blockchain address associated with the reward provider (e.g., a user, the avatar server, a health and fitness system, an insurance provider (e.g., providing a discounted rate as a reward), a retailer system 1720, an XR system, 1722, etc.) to the blockchain address (or addresses) of the reward receiver. In embodiments, in which the NFT reward is minted, the minting component 1816 can mint the NFT reward based on satisfaction of the smart contract criteria and record the minted NFT in the reward receiver's account, that is the reward receivers name/digital wallet account and/or corresponding blockchain address.

In this regard, minting an NFT refers to tokening (e.g., with a unique, non-fungible toke) a real-world or digital asset (e.g., a digital collectible, a digital file, a digital object corresponding coupon or certificate redeemable for real-world goods and/or services, etc.) and recording ownership of that asset to a specific entity or user on a blockchain (e.g., blockchain 1704 or another blockchain). In some embodiments, the minting component 1816 can mint reward NFTs to public marketplace platforms for users that have digital wallets compatible with those forums (e.g., SuperRare®, Nifty Gateway®, Makerspace®, Foundation® . . . ) and issue a token to guarantee its authenticity. In some implementations, where applicable for minting NFTs, the minting component 304 can pay “gas fees” to initiate the minting transaction on the blockchain (e.g., blockchain 1704 or another blockchain) and publish reward digital asset as an NFT on a marketplace. Once the digital asset has been minted on a marketplace as an NFT, the minting component 1816 can restrict minting of that digital asset on another platform. In this regard, minting is how a set a digital asset (or real-world asset) becomes a part of a blockchain—a ledger that is unchangeable and tamper-proof. Similar to the way that metal coins are minted and added into circulation, NFTs are also tokens that get “minted” once they are created. The asset is represented as an NFT, e.g., so it can then be purchased and traded in the market and digitally tracked as it is resold, gifted, or collected again in the future.

The minting component 1816 can “Sign” the minting transaction, which will link the NFT to the blockchain address of the NFT reward receiver, accessible via their corresponding digital wallet, which allows others to verify the digital assets belong to the owner of the address and/or digital wallet, and ensures that the owner of the digital wallet is listed as the owner of the digital asset represented by the minted NFT.

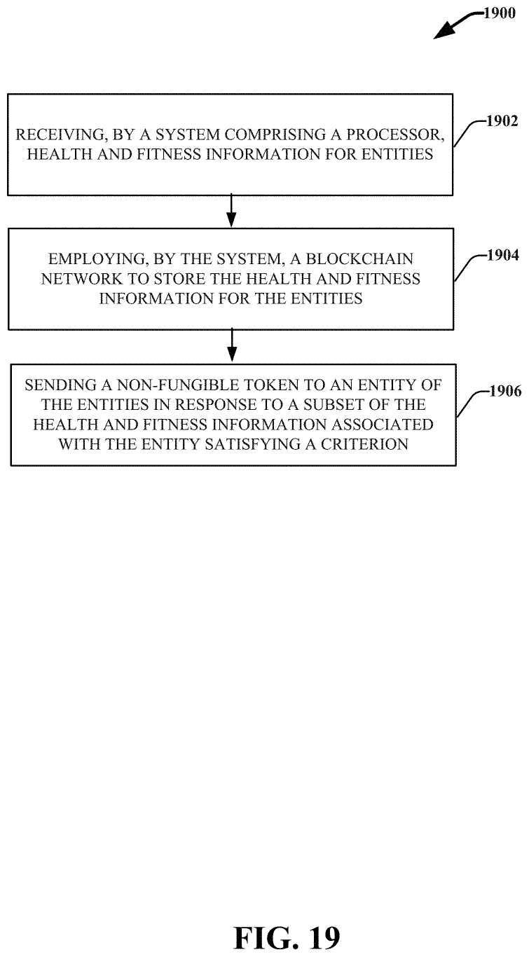

FIG. 19 illustrates an example computer-implemented method 1900 for tracking and rewarding health and fitness activities of users using blockchain technology, in accordance with one or more embodiments described herein. Method 1900 comprises an example computer-implemented process that can be executed via one or more devices of system 1700 using one or more of the machine executable components 1800. Repetitive description of like elements employed in respective embodiments is omitted for sake of brevity.

In accordance with method 1900, at 1902 a system comprising a processor (e.g., system 1700) receives health and fitness information for entities, such as users or groups of users (e.g., via reception component 1802). At 1904, the system can employ a blockchain network to store the health and fitness information for the entities (e.g., blockchain network 1702 and blockchain 1704). For example, in some embodiments, the receiving step can correspond to reception of the health and fitness information at devices and/or systems external to the blockchain network (e.g., one or more sensors 104, one or more IFD 118, one or more client devise 106, avatar server 116, one or more health and fitness systems 1716, and the like) that in turn provide the health and fitness information to the blockchain network 1702 via suitable wired or wireless communication technologies. In other embodiments, the receiving step can correspond to reception of the health and fitness information by one or more devices (e.g., nodes 1703₁₋ₖ and/or one or more servers of the blockchain network 1702). In some implementations of either of these embodiments, the external devices and/or systems (e.g., avatar server 116) can be configured process data signals captured via the one or more sensors 104, an IFD 118 and/or image data captured of the user in association with monitoring the user to generate the corresponding health and fitness information. For example, the health and fitness information can include information regarding performance of fitness activities, including information regarding activity type, exercises performed (e.g., types of moves performed, types of equipment used), and performance metrics associated with the activities and/or exercises performed (e.g., regarding amount, frequency, duration, level, timing, intensity, form correctness, calories burned, heart rate, etc.). The health and fitness information can also include information regarding location, distance and route where applicable (e.g., for running, biking, swimming, rowing, etc. activities that involve movement over distances). The health and fitness information can also include body measurement data, such as weight, BMI, body fat index, dimensions of body parts, muscle mass, etc.). At 1906, the system can further send an NFR to an entity of the entities in response to a subset of the health and fitness information associated with the entity satisfying a criterion (e.g., via reward provision component 1814).

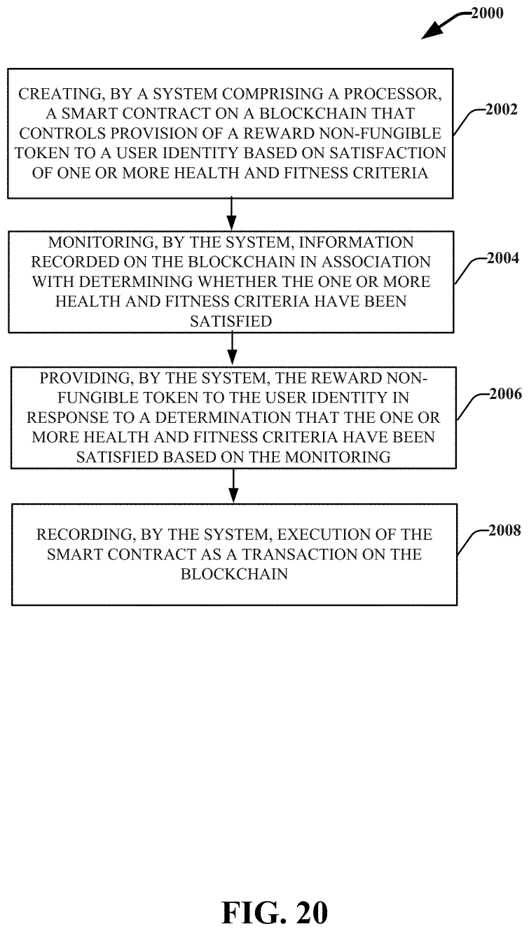

FIG. 20 illustrates another example computer-implemented method 2000 for tracking and rewarding health and fitness activities of users using blockchain technology, in accordance with one or more embodiments described herein. Method 2000 comprises an example computer-implemented process that can be executed via one or more devices of system 1700 using one or more of the machine executable components 1800. Repetitive description of like elements employed in respective embodiments is omitted for sake of brevity.

In accordance with method 2000, at 2002 a system comprising a processor (e.g., system 1700) creates (e.g., encodes, stores, generates, etc.) a smart contract on a blockchain (e.g., blockchain 1704) that controls provision of a reward NFT to a user identity (e.g., associated with their corresponding blockchain address) based on satisfaction of one or more health and fitness criteria (e.g., via reward management component 1812. For example, in some embodiments, the reward management component 1812 can receive input (e.g., via a suitable user interface) that defines terms and conditions of the smart contract regarding the health and fitness criteria to be satisfied, the user or users eligible for the reward (e.g., a specific user, any user satisfying the criteria, a subgroup of users satisfying the criteria, etc.) and the specific NFT reward to be provided (e.g., minted and/or transferred from the reward provider's address to the reward receiver address). Based on reception of this information the reward management component 1812 can create and record the smart contract on the blockchain 1704 (e.g., as a blockchain record).

At 2002, the system can further monitor (e.g., via the reward management component 1812 and/or via the nodes 1703₁₋ₖ in accordance with the terms and conditions defined by the smart contract) information recorded on the blockchain (e.g., blockchain 1704) in association with determining whether the one or more health and fitness criteria have been satisfied (e.g., using a consensus model or another suitable mode). In some implementations of these embodiments, if the contract expires and/or information has been received that indicates the contract is no longer valid or becomes void, the system can declare the contract terminated, record termination of the contract on the blockchain. The system can further end the monitoring process relative to the contract. For example, assume a contract indicates a user will be rewarded with a particular reward NFT if the user completes at least three workouts a week for three weeks. If information has been received following week 1 or week 2 that the user only completed one or two workouts that week, the system can declare the contract null and void and stop the monitoring processes. At 2006, the system can provide the reward NFT to the user identity (e.g., to their associated blockchain address and/or digital wallet) in response to a determination that the one or more health and fitness criteria have been satisfied based on the monitoring (e.g., via reward provision component 1814). At 2008, the system further records execution of the smart contract as a transaction on the blockchain (e.g., via blockchain recording component 1804).

Computing Architecture

As discussed above, the distributed ledger in a blockchain framework is stored, maintained, and updated in a peer-to-peer network. In one example the distributed ledger maintains a number of blockchain transactions. FIG. 21 shows an example system 2100 for facilitating a blockchain transaction, such as transfer or exchange of NFTs between entities, a transfer or exchange of cryptocurrency between entities and the like. The system 2100 includes a first client device 2120, a second client device 2125, a first server 2150, and an Internet of Things (IoT) device 2155 interconnected via a network 2140. The first client device 2120, the second client device 2125, the first server 2150 may be a computing device. The IoT device 2155 may comprise any of a variety of devices including sensors (e.g., sensors 104), intelligent fitness equipment (e.g., IFD 118), wearable devices, cameras (e.g., visual capture device 110), home appliances, embedded electronics, software, actuators, thermostats, light bulbs, door locks, refrigerators, RFID implants, RFID tags, pacemakers, smart home devices, trackers, pumps, POS devices, and stationary and mobile communication devices along with connectivity hardware configured to connect and exchange data. The network 2140 may be any of a variety of available networks, such as the Internet, and represents a worldwide collection of networks and gateways to support communications between devices connected to the network 2140. The system 2100 may also comprise one or more distributed or peer-to-peer (P2P) networks, such as a first, second, and third blockchain network 2130a-c (generally referred to as blockchain networks 2130). As shown in FIG. 21, the network 2140 may comprise the first and second blockchain networks 2130a and 2130b. The third blockchain network 2130c may be associated with a private blockchain as described below with reference to FIG. 21, and is thus, shown separately from the first and second blockchain networks 2130a and 2130b. In various embodiments, blockchain network 1702 may correspond to one or more of blockchain network 2130a, blockchain network 2130b, and/or blockchain network 2130c. Each blockchain network 2130 may comprise a plurality of interconnected devices (or nodes, such as nodes 1703₁₋ₖ). As discussed above, a ledger, or blockchain, is a distributed database for maintaining a growing list of records comprising any type of information, including tracked health and fitness information, NFTs, and smart contracts related to provision of rewards (e.g., NFTs and/or cryptocurrency) based on defined achievement criteria for the health and fitness information. A blockchain, may be stored at least at multiple nodes (or devices) of the one or more blockchain networks 2130.

In one example, a blockchain based transaction may generally involve a transfer of data or value between entities, such as the first user 2110 of the first client device 2120 and the second user 2115 of the second client device 2125 in FIG. 21. The server 2150 may include one or more applications, for example, a transaction application configured to facilitate the transaction between the entities by utilizing a blockchain associated with one of the blockchain networks 2130. As an example, the first user 2110 may request or initiate a transaction with the second user 2115 via a user application executing on the first client device 2120. The transaction may be related to a transfer of value or data from the first user 2110 to the second user 2115. The first client device 2120 may send a request of the transaction to the server 2150. The server 2150 may send the requested transaction to one of the blockchain networks 2130 to be validated and approved as discussed below.

Blockchain Network

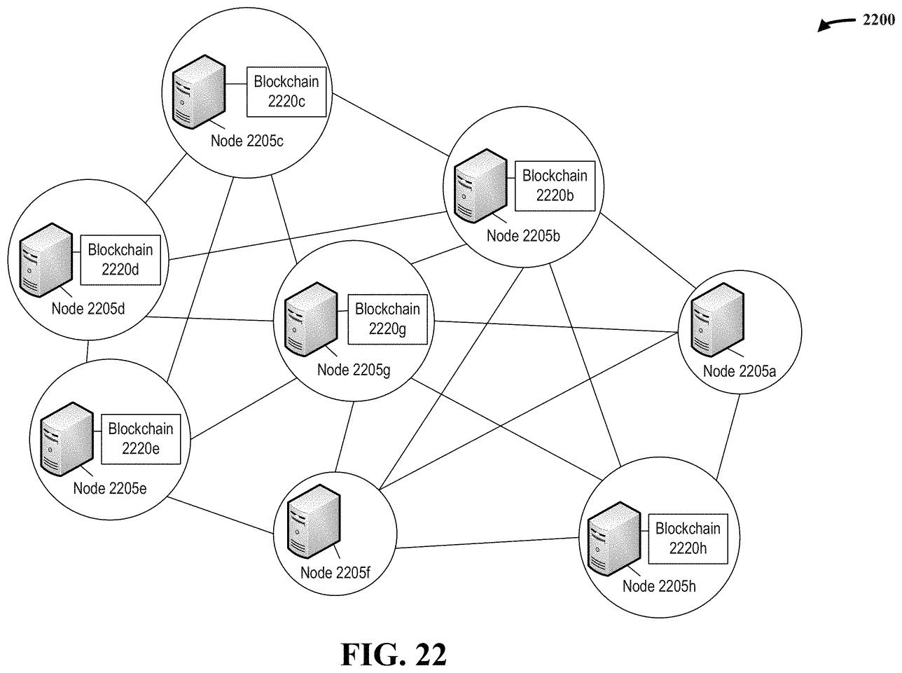

FIG. 22 shows an example blockchain network 2200 comprising a plurality of interconnected nodes or devices 2205a-h (generally referred to as nodes 2205). In one or more embodiments, blockchain network 1702 may correspond to blockchain network 2200 and the nodes 1703₁₋ₖ of blockchain network 1702 may correspond to nodes 2205. Each of the nodes 2105 may comprise a computing device. Although FIG. 22 shows a single device 2205, each of the nodes 2205 may comprise a plurality of devices (e.g., a pool). The blockchain network 2200 may be associated with a blockchain 2220. In various embodiments, blockchain 1704 may correspond to blockchain 2220. Some or all of the nodes 2205 may replicate and save an identical copy of the blockchain 2220. For example, FIG. 22 shows that the nodes 2205b-e and 2205g-h store copies of the blockchain 2220. The nodes 2205b-e and 2205g-h may independently update their respective copies of the blockchain 2220 as discussed below.

Blockchain Node Types

Blockchain nodes, for example, the nodes 2205, may be full nodes or lightweight nodes. Full nodes, such as the nodes 2205b-e and 2205g-h, may act as a server in the blockchain network 2200 by storing a copy of the entire blockchain 2220 and ensuring that transactions posted to the blockchain 2220 are valid. The full nodes 2205b-e and 2205g-h may publish new blocks on the blockchain 2220. Lightweight nodes, such as the nodes 2205a and 2205f, may have fewer computing resources than full nodes. For example, IoT devices often act as lightweight nodes. The lightweight nodes may communicate with other nodes 2205, provide the full nodes 2205b-e and 2205g-h with information, and query the status of a block of the blockchain 2220 stored by the full nodes 2205b-e and 2205g-h. In this example, however, as shown in FIG. 22, the lightweight nodes 2205a and 2205f may not store a copy of the blockchain 2220 and thus, may not publish new blocks on the blockchain 2220.

Blockchain Network Types

The blockchain network 2200 and its associated blockchain 2220 may be public (permissionless), federated or consortium, or private. If the blockchain network 2200 is public, then any entity may read and write to the associated blockchain 2220. However, the blockchain network 2200 and its associated blockchain 2220 may be federated or consortium if controlled by a single entity or organization (e.g., or group of authorized entities/organizations). Further, any of the nodes 2205 with access to the Internet may be restricted from participating in the verification of transactions on the blockchain 2220. The blockchain network 2200 and its associated blockchain 2220 may be private (permissioned) if access to the blockchain network 2200 and the blockchain 2220 is restricted to specific authorized entities, for example organizations or groups of individuals associated with system 1700. Moreover, read permissions for the blockchain 2220 may be public or restricted while write permissions may be restricted to a controlling or authorized entity.

Blockchain

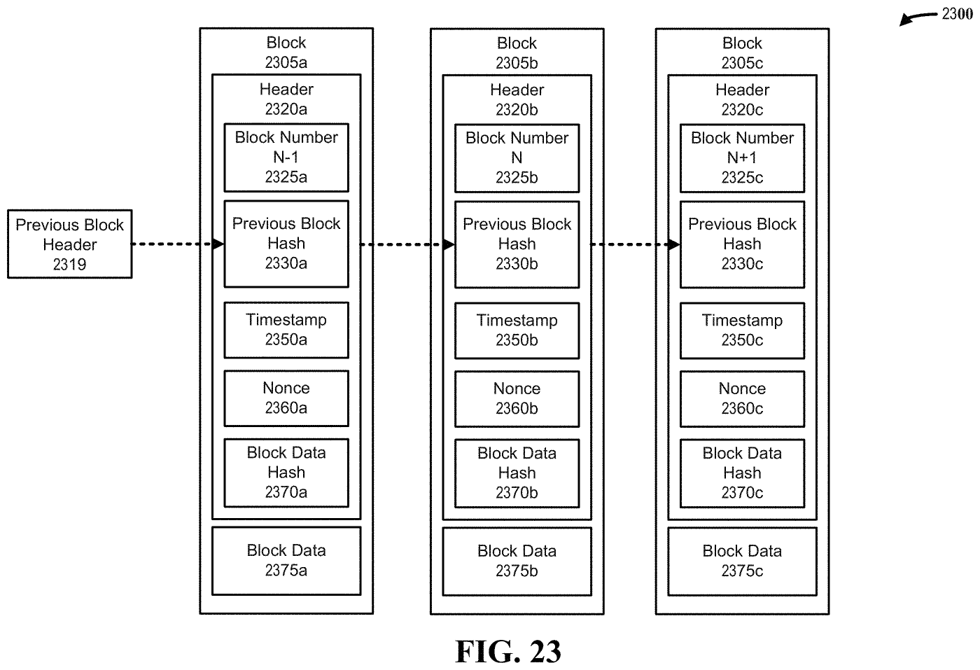

As discussed above, a blockchain 2220 (corresponding to blockchain 1704) may be associated with a blockchain network 2200 (corresponding to blockchain network 1702). FIG. 23 shows an example blockchain 2300. In various embodiments, the blockchain 1704 may correspond to blockchain 2300. The blockchain 2300 may comprise a plurality of blocks 2305a, 2305b, and 2305c (generally referred to as blocks 2305). The blockchain 2300 comprises a first block (not shown), sometimes referred to as the genesis block. Each of the blocks 2305 may comprise a record of one or a plurality of submitted and validated blockchain records (e.g., of health and fitness information for users, assignment of NFTs, etc.,) and/or transactions (e.g., exchange/transfer of NFTs and other digital assets). The blocks 2305 of the blockchain 2300 may be linked together and cryptographically secured. In some cases, the post-quantum cryptographic algorithms that dynamically vary over time may be utilized to mitigate ability of quantum computing to break present cryptographic schemes. Examples of the various types of data fields stored in a blockchain block are provided below. A copy of the blockchain 2300 may be stored locally, in the cloud, on grid, for example by the nodes 2205b-e and 2205g-h, as a file or in a database.

Blocks

Each of the blocks 2305 may comprise one or more data fields. The organization of the blocks 2305 within the blockchain 2300 and the corresponding data fields may be implementation specific. As an example, the blocks 2305 may comprise a respective header 2320a, 2320b, and 2320c (generally referred to as headers 2320) and block data 2375a, 2375b, and 2375c (generally referred to as block data 2375). The headers 2320 may comprise metadata associated with their respective blocks 2305. For example, the headers 2320 may comprise a respective block number 2325a, 2325b, and 2325c. As shown in FIG. 23, the block number 2325a of the block 2305a is N−1, the block number 2325b of the block 2305b is N, and the block number 2325c of the block 2305c is N+1. The headers 2320 of the blocks 2305 may include a data field comprising a block size (not shown).

The blocks 2305 may be linked together and cryptographically secured. For example, the header 2320b of the block N (block 2305b) includes a data field (previous block hash 2330b) comprising a hash representation of the previous block N−1's header 2320a. The hashing algorithm utilized for generating the hash representation may be, for example, a secure hashing algorithm which results in an output of a fixed length. In this example, the hashing algorithm is a one-way hash function, where it is computationally difficult to determine the input to the hash function based on the output of the hash function. Additionally, the header 2320c of the block N+1 (block 2305c) includes a data field (previous block hash 2330c) comprising a hash representation of block N's (block 2305b) header 2320b.

The headers 2320 of the blocks 2305 may also include data fields comprising a hash representation of the block data, such as the block data hash 2370a-c. The block data hash 2370a-c may be generated, for example, by a Merkle tree and by storing the hash or by using a hash that is based on all of the block data. The headers 2320 of the blocks 2305 may comprise a respective nonce 2360a, 2360b, and 2360c. In some implementations, the value of the nonce 2360a-c is an arbitrary string that is concatenated with (or appended to) the hash of the block. The headers 2320 may comprise other data, such as a difficulty target.

The blocks 2305 may comprise a respective block data 2375a, 2375b, and 2375c (generally referred to as block data 2375). The block data 2375 may comprise a record of validated data records (e.g., of health and fitness data) and/or transactions that have also been integrated into the blockchain 2200 via a consensus model (described below). As discussed above, the block data 2375 may include a variety of different types of data in addition to validated transactions. Block data 2375 may include any data, such as text, audio, video, image, or file, that may be represented digitally and stored electronically.

Blockchain Transaction

In one example, a blockchain based transaction may generally involve a transfer of data or value or an interaction between entities and described in more detail below. Referring back to FIG. 21, the server 2150 may include one or more applications, for example, a transaction application configured to facilitate a blockchain transaction between entities. The entities may include users, devices, etc. The first user 2110 may request or initiate a transaction with the second user 2115 via a user application executing on the first client device 2120. The transaction may be related to a transfer of value or data from the first user 2110 to the second user 2115. The value or data may represent money, a contract, property, records, rights, status, supply, demand, alarm, trigger, or any other asset that may be represented in digital form. The transaction may represent an interaction between the first user 2110 and the second user 2115.

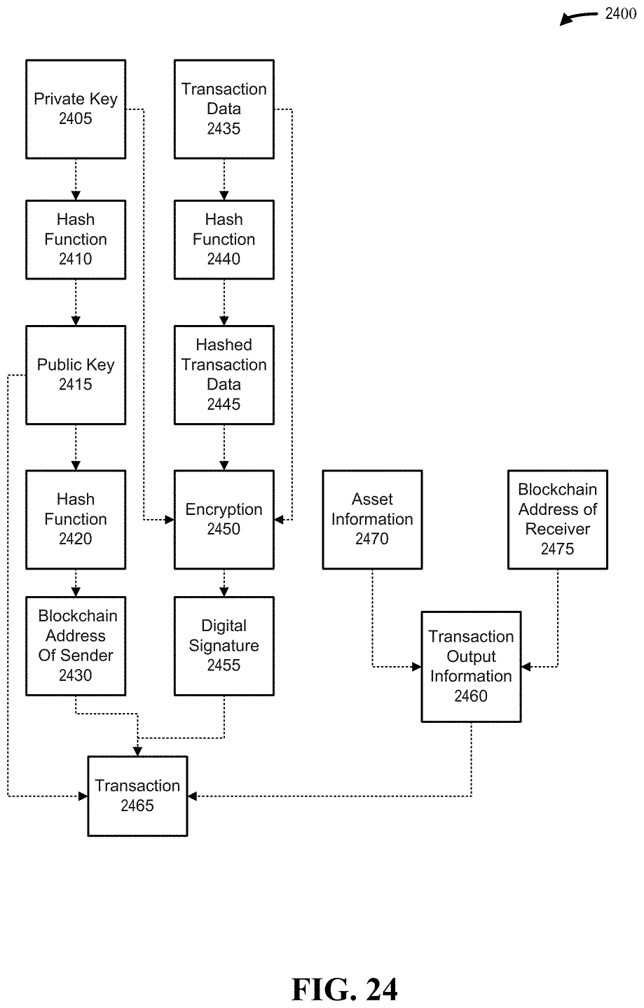

FIG. 24 is a diagram of a transaction 2465 generated by the transaction application. The transaction 2465 may include a public key 2415, a blockchain address 2430 associated with the first user 2110, a digital signature 2455, and transaction output information 2460. The transaction application may derive a public key 2415 from a private key 2405 of the first user 2110 by applying a cryptographic hash function 2410 to the private key 2405. The cryptographic hash function 2410 may be based on AES, SHA-2, SHA-3, RSA, ECDSA, ECDH (elliptic curve cryptography), or DSA (finite field cryptography), although other cryptographic models may be utilized. More information about cryptographic algorithms may be found in Federal Information Processing Standards Publication (FIPS PUB 180-3), Secure Hash Standard. The transaction application may derive an address or identifier for the first user 2110, such as the blockchain address 2430, by applying a hash function 2420 to the public key 2415. Briefly, a hash function is a function that may be used for mapping arbitrary size data to fixed size data. The value may also be referred to as a digest, a hash value, a hash code, or a hash. In order to indicate that the first user 2110 is the originator of the transaction 2465, the transaction application may generate the digital signature 2455 for the transaction data 2435 using the private key 2405 of the first user 2110. The transaction data 2435 may include information about the assets to be transferred and a reference to the sources of the assets, such as previous transactions in which the assets were transferred to the first user 2410 or an identification of events that originated the assets. Generating the digital signature 2455 may include applying a hash function 2440 to the transaction data 2435 resulting in hashed transaction data 2445. The hashed transaction data 2445 and the transaction data 2435 may be encrypted (via an encryption function 2450) using the private key 2405 of the first user 2110 resulting in the digital signature 2455. The transaction output information 2460 may include asset information 2470 and an address or identifier for the second user 2115, such as the blockchain address 2475. The transaction 2465 may be sent from the first client device 2125 to the server 2150.

The specific type of cryptographic algorithm being utilized may vary dynamically based on various factors, such as a length of time, privacy concerns, etc. For example, the type of cryptographic algorithm being utilized may be changed yearly, weekly, daily, etc. The type of algorithms may also change based on varying levels of privacy. For example, an owner of content may implement a higher level of protection or privacy by utilizing a stronger algorithm.

Blockchain Addresses

A blockchain network may utilize blockchain addresses to indicate an entity using the blockchain or start and end points in the transaction. For example, a blockchain address for the first user 2110, shown in FIG. 24 as the blockchain address of sender 2330, may include an alphanumeric string of characters derived from the public key 2415 of the first user 2110 based on applying a cryptographic hash function 2420 to the public key 2415. The methods used for deriving the addresses may vary and may be specific to the implementation of the blockchain network. In some examples, a blockchain address may be converted into a QR code representation, barcode, token, or other visual representations or graphical depictions to enable the address to be optically scanned by a mobile device, wearables, sensors, cameras, etc. In addition to an address or QR code, there are many ways of identifying individuals, objects, etc. represented in a blockchain. For example, an individual may be identified through biometric information such as a fingerprint, retinal scan, voice, facial id, temperature, heart rate, gestures/movements unique to a person etc., and through other types of identification information such as account numbers, home address, social security number, formal name, etc.

Broadcasting Transactions and Other Data Entries

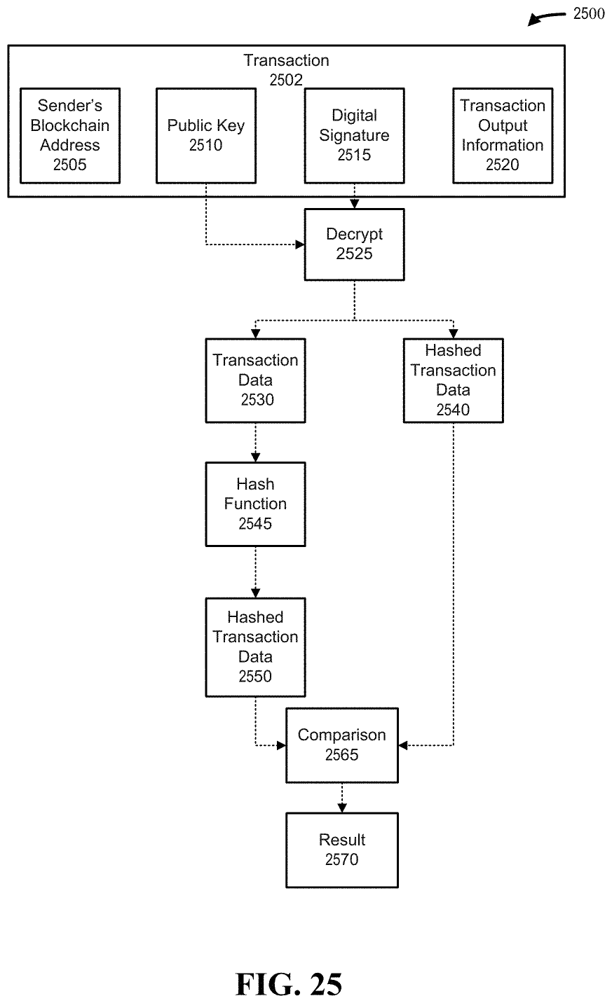

The server 2150 may receive transactions and data entries from users and entities (e.g., systems/devices) of the blockchain network 2130. The transactions may be submitted to the server 2150 via desktop applications, smartphone applications, digital wallet applications, web services, or other software applications. The server 2150 may send or broadcast the transactions to the blockchain network 2130. FIG. 25 shows an example transaction 2502 broadcast by the server 2150 to the blockchain network 2130. The transaction 2502 may be broadcast to multiple nodes of the blockchain network 2130. Typically, once the transaction 2502 is broadcast or submitted to the blockchain network 2130, it may be received by one or more of the nodes (e.g., nodes 2505, nodes 1703₁₋ₖ, etc.). Once the transaction 2502 is received by the one or more initial receiving nodes of the blockchain network 2130, it may be propagated by the initial receiving nodes to other nodes of the blockchain network 1230.

A blockchain network may operate according to a set of rules. The rules may specify conditions under which a node may accept a transaction and/or data entry, a type of transaction and/or data entry that a node may accept, a type of compensation that a node receives for accepting and processing a transaction and/or data entry, etc. For example, a node may accept a transaction and/or data entry based on a transaction history, a source verification, reputation, computational resources, relationships with service providers, etc. The rules may specify conditions for broadcasting a transaction to a node. For example, a transaction may be broadcast to one or more specific nodes based on criteria related to the node's geography, history, reputation, market conditions, docket/delay, technology platform. The rules may be dynamically modified or updated (e.g., turned on or off) to address issues such as latency, scalability and security conditions. A transaction may be broadcast to a subset of nodes as a form of compensation to entities associated with those nodes (e.g., through receipt of compensation for adding a block of one or more transactions to a blockchain).

Transaction Validation— User Authentication and Transaction Data Integrity

With reference to FIGS. 22 and 25, not all the full nodes 2205 may receive the broadcasted transaction 2502 at the same time, due to issues such as latency. Additionally, not all of the full nodes 2205 that receive the broadcasted transaction 2502 may choose to validate the transaction 2502. A node 2205 may choose to validate specific transactions, for example, based on transaction fees associated with the transaction 2502. The transaction 2502 may include a blockchain address 2505 for the sender, a public key 2510, a digital signature 2515, and transaction output information 2520. The node 2505 may verify whether the transaction 2502 is legal or conforms to a pre-defined set of rules. The node 2505 may also validate the transaction 2502 based on establishing user authenticity and transaction data integrity. User authenticity may be established by determining whether the sender indicated by the transaction 2502 is in fact the actual originator of the transaction 2502. User authenticity may be proven via cryptography, for example, asymmetric-key cryptography using a pair of keys, such as a public key and a private key. Additional factors may be considered when establishing user authenticity, such as user reputation, market conditions, history, transaction speed, etc. Data integrity of the transaction 2502 may be established by determining whether the data associated with the transaction 2502 was modified in any way. Referring back to FIGS. 21 and 24, when the transaction application creates the transaction 2465, it may indicate that the first user 2110 is the originator of the transaction 2465 by including the digital signature 2455.

With reference again to FIGS. 22 and 25, at 2525 the node 2205 may decrypt the digital signature 2515 using the public key 2510. A result of the decryption, the node may include hashed transaction data 2540 and transaction data 2530. The node 2205 may generate hashed transaction data 2550 based on applying a hash function 2545 to the transaction data 2530. The node 2205 may perform a comparison 2565 between the first hashed transaction data 2540 and the second hashed transaction data 2550. If the result 2570 of the comparison 2565 indicates a match, then the data integrity of the transaction 2502 may be established and node 2205 may indicate that the transaction 2502 has been successfully validated. Otherwise, the data of the transaction 2502 may have been modified in some manner and the node 2205 may indicate that the transaction 2502 has not been successfully validated.

With reference Each full node 2505 may build its own block and add validated transactions to that block. Thus, the blocks of different full nodes 2205 may comprise different validated transactions. As an example, a full node 2205a may create a first block comprising transactions “A,” “B,” and “C.” Another full node 2205b may create a second block comprising transactions “C,” “D,” and “E.” Both blocks may include valid transactions. However, only one block may get added to the blockchain, otherwise the transactions that the blocks may have in common, such as transaction “C” may be recorded twice leading to issues such as double-spending when a transaction is executed twice. One problem that may be seen with the above example is that transactions “C,” “D,” and “E” may be overly delayed in being added to the blockchain. This may be addressed a number of different ways as discussed below.

Securing Keys

Private keys, public keys, and addresses may be managed and secured using software, such as a digital wallet. Private keys may also be stored and secured using hardware. The digital wallet may also enable the user to conduct transactions and manage the balance. The digital wallet may be stored or maintained online or offline, and in software or hardware or both hardware and software. Without the public/private keys, a user has no way to prove ownership of assets. Additionally, anyone with access a user's public/private keys may access the user's assets. While the assets may be recorded on the blockchain, the user may not be able to access them without the private key.

Tokens

A token, such as an NFT, may refer to an entry in the blockchain that belongs to a blockchain address. The entry may comprise information indicating ownership of an asset. The token may represent money, a contract, property, records, access rights, status, supply, demand, alarm, trigger, reputation, ticket, or any other asset that may be represented in digital form. For example, a token may refer to an entry related to cryptocurrency that is used for a specific purpose or may represent ownership of a real-world asset, such as Fiat currency, real-estate, good and services, as well as other digital assets (e.g., digital coupons, digital collectables, etc.). Token contracts refer to cryptographic tokens that represent a set of rules that are encoded in a smart contract. The person that owns the private key corresponding to the blockchain address may access the tokens at the address. Thus, the blockchain address may represent an identity of the person that owns the tokens. Only the owner of the blockchain address may send the token to another person. The tokens may be accessible to the owner via the owner's wallet. The owner of a token may send or transfer the token to a user via a blockchain transaction. For example, the owner may sign the transaction corresponding to the transfer of the token with the private key. When the token is received by the user, the token may be recorded in the blockchain at the blockchain address of the receiving user.

Establishing User Identity

While a digital signature may provide a link between a transaction and an owner of assets being transferred, it may not provide a link to the real identity of the owner. In some cases, the real identity of the owner of the public key corresponding to the digital signature may need to be established. The real identity of an owner of a public key may be verified, for example, based on biometric data, passwords, personal information, etc. Biometric data may comprise any physically identifying information such as fingerprints, face and eye images, voice sample, DNA, human movement, gestures, gait, expressions, heart rate characteristics, temperature, etc.

Publishing and Validating a Block

As discussed above, full nodes 2205 may each build their own blocks that include different transactions. A node may build a block by adding validated transactions to the block until the block reaches a certain size that may be specified by the blockchain rules. However, only one of the blocks may be added to the blockchain. The block to be added to the blockchain and the ordering of the blocks may be determined based on a consensus model. In a proof of work model, both nodes may compete to add their respective block to the blockchain by solving a complex mathematical puzzle. For example, such a puzzle may include determining a nonce, as discussed above, such that a hash (using a predetermined hashing algorithm) of the block to be added to the blockchain (including the nonce) has a value that meets a range limitation. If both nodes solve the puzzle at the same time, then a “fork” may be created. When a full node 2205 solves the puzzle, it may publish its block to be validated by the validation nodes 2205 of the blockchain network 2130.

In a proof of work consensus model, a node validates a transaction, for example, by running a check or search through the current ledger stored in the blockchain. The node will create a new block for the blockchain that will include the data for one or more validated transactions (see, e.g., block 2375 of FIG. 23). In a blockchain implementation such as Bitcoin, the size of a block is constrained. Referring back to FIG. 23, in this example, the block will include a Previous Block Hash 2330 representing a hash of what is currently the last block in the blockchain. The block may also include a hash 2370 of its own transaction data (e.g., a so-called Merkle hash). According to a particular algorithm, all or selected data from the block may be hashed to create a final hash value. According to an embodiment of the proof of work model, the node will seek to modify the data of the block so that the final hash value is less than a preset value. This is achieved through addition of a data value referred to as a nonce 2360. Because final hash values cannot be predicted based on its input, it is not possible to estimate an appropriate value for the nonce 2360 that will result in a final hash value that is less than the pre-set value. Accordingly, in this embodiment, a computationally-intensive operation is needed at the node to determine an appropriate nonce value through a “brute force” trial-and-error method. Once a successful nonce value is determined, the completed block is published to the blockchain network for validation. If validated by a majority of the nodes in the block chain network, the completed block is added to the blockchain at each participating node. When a node's block is not added to the blockchain, the block is discarded and the node proceeds to build a new block. The transactions that were in the discarded block may be returned to a queue and wait to be added to a next block. When a transaction is discarded or returned to the queue, the assets associated with the discarded transaction are not lost, since a record of the assets will exist in the blockchain. However, when a transaction is returned to the queue it causes a delay in completing the transaction. Reducing the time to complete a transaction may be important. A set of blockchain rules, or renumeration/compensation for a node to process the returned transaction may determine how a returned transaction is to be treated going forward. When a transaction is put into a pool then it can have a priority level but then a rule may indicate that the transaction priority level must exceed a threshold level. The priority level of a returned or discarded transaction may be increased. Another way to reduce the time to complete a transaction is to have the system, service provider, participant in the transaction, or merchant pay additional incentive for nodes to process a returned transaction. As an example, a service provider may identify a network of preferred miners based on geography or based on a volume discount perspective. The time to complete a transaction may be optimized by routing a returned transaction to specific preferred nodes. A transaction may be associated with an address that limits which of the preferred nodes will get to process the transaction if it is returned due to its inclusion in a discarded block. A value may be associated with the transaction so that it goes to preferred miners in a specific geographic location. Additionally, returned transactions may be processed based on pre-set rules. For example, a rule may indicate a commitment to process a specific number of returned transactions to receive additional incentive or compensation.

Blockchain Confirmations

After a block comprising a transaction is added to a blockchain, a blockchain confirmation may be generated for the transaction. The blockchain confirmation may be a number of blocks added to the blockchain after the block that includes the transaction. For example, when a transaction is broadcast to the blockchain, there will be no blockchain confirmations associated with the transaction. If the transaction is not validated, then the block comprising the transaction will not be added to the blockchain and the transaction will continue to have no blockchain confirmations associated with it. However, if a block comprising the transaction is validated, then each of the transactions in the block will have a blockchain confirmation associated with the transaction. Thus, a transaction in a block will have one blockchain confirmation associated with it when the block is validated. When the block is added to the blockchain, each of the transactions in the block will have two blockchain confirmations associated with it. As additional validated blocks are added to the blockchain, the number of blockchain confirmations associated with the block will increase. Thus, the number of blockchain confirmations associated with a transaction may indicate a difficulty of overwriting or reversing the transaction. A higher valued transaction may require a larger number of blockchain confirmations before the transaction is executed.

Consensus Models

As discussed above, a blockchain network may determine which of the full nodes 2205 publishes a next block to the blockchain. In a permissionless blockchain network, the nodes 2505 may compete to determine which one publishes the next block. A node 2205 may be selected to publish its block as the next block in the blockchain based on consensus model. For example, the selected or winning node 2505 may receive a reward, such as a transaction fee, for publishing its block, for example. Various consensus models may be used, for example, a proof of work model, a proof of stake model, a delegated proof of stake model, a round robin model, proof of authority or proof of identity model, and proof of elapsed time model.

In a proof of work model, a node may publish the next block by being the first to solve a computationally intensive mathematical problem (e.g., the mathematical puzzle described above). The solution serves as “proof” that the node expended an appropriate amount of effort in order to publish the block. The solution may be validated by the full nodes before the block is accepted. The proof of work model, however, may be vulnerable to a 51% attack described below. The proof of stake model is generally less computationally intensive that the proof of work model. Unlike the proof of work model which is open to any node having the computational resources for solving the mathematical problem, the proof of stake model is open to any node that has a stake in the system. The stake may be an amount of cryptocurrency that the blockchain network node (user) may have invested into the system. The likelihood of a node publishing the next block may be proportional to its stake. Since this model utilizes fewer resources, the blockchain may forego a reward as incentive for publishing the next block. The round robin model is generally used by permissioned blockchain networks. Using this model, nodes may take turns to publish new blocks. In the proof of elapsed time model, each publishing node requests a wait time from a secure hardware within their computer system. The publishing node may become idle for the duration of the wait time and then creates and publishes a block to the blockchain network. As an example, in cases where there is a need for speed and/or scalability (e.g., in the context of a corporate environment), a hybrid blockchain network may switch to be between completely or partially permissioned and permissionless. The network may switch based on various factors, such as latency, security, market conditions, etc.

Blockchain Based Application: Cryptocurrency

Cryptocurrency is a medium of exchange that may be created and stored electronically in a blockchain, such as the blockchain 1704 in FIG. 17, blockchain 2220 in FIG. 22 and the like. Bitcoin is one example of cryptocurrency, however there are several other cryptocurrencies. Various encryption techniques may be used for creating the units of cryptocurrency and verifying transactions. As an example, the first user 2110 may own 10 units of a cryptocurrency. The blockchain network 2130a may include a record indicating that the first user 2110 owns the 10 units of cryptocurrency. The first user 2110 may initiate a transfer of the 10 units of cryptocurrency to the second user 2115 via a wallet application executing on the first client device 2120. The wallet application may store and manage a private key of the first user 2110. Examples of the wallet device include a personal computer, a laptop computer, a smartphone, a personal data assistant (PDA), etc.

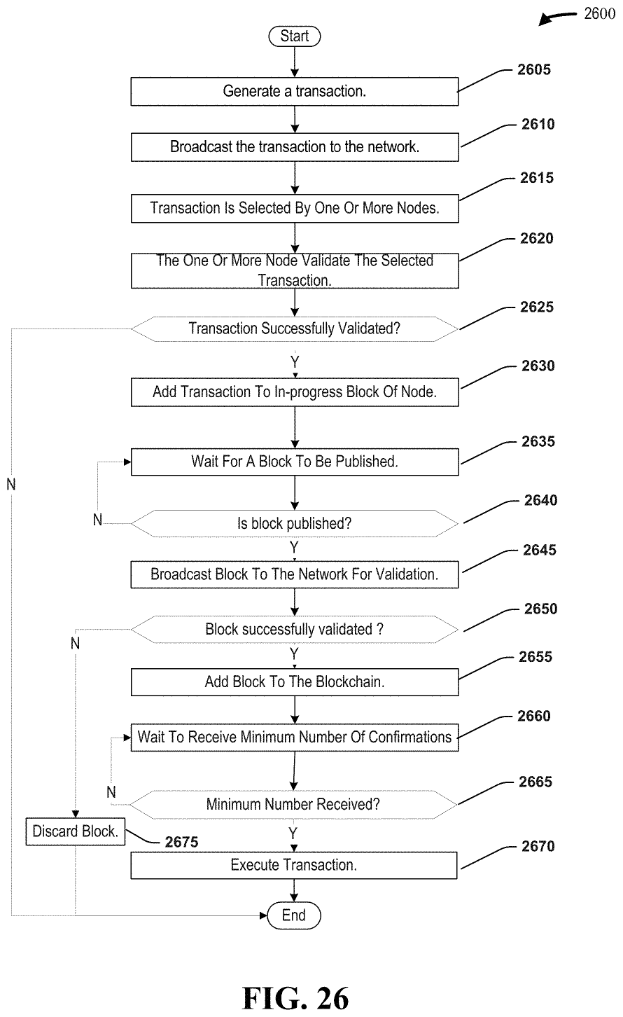

FIG. 26 is a flow diagram showing steps of an example method 2600 for performing a blockchain transaction between entities, such as the first user 2110 of the first client device 2120 and the second user 2115 of the second client device 2125 in FIG. 21. The steps of the method 2600 may be performed by any of the computing devices shown in FIG. 21. Alternatively or additionally, some or all of the steps of the method 2600 may be performed by one or more other computing devices. Steps of the method 2600 may be modified, omitted, and/or performed in other orders, and/or other steps added.

At step 2605, the wallet application may generate transaction data for transferring the 10 units of cryptocurrency from the first user 2110 to the second user 2115. The wallet application may generate a public key for the transaction using the private key of the first user 2110. In order to indicate that the first user 2110 is the originator of the transaction, a digital signature may also be generated for the transaction using the private key of the first user 2110. As discussed with reference to FIG. 24, the transaction data may include information, such as a blockchain address of the sender 24230, the digital signature 2455, transaction output information 2460, and the public key of the sender 2415. The transaction data may be sent to the server 2150 from the first client device 2125.

The server 2150 may receive the transaction data from the first client device 2125. At step 1610, the server 2150 may broadcast the transaction to the blockchain network 2130. The transaction may be received by one or more nodes 2205 of the blockchain network 2130. At step 2615, upon receiving the transaction, a node 2205 may choose to validate the transaction, for example, based on transaction fees associated with the transaction. If the transaction is not selected for validation by any of the nodes 2205, then the transaction may be placed in a queue and wait to be selected by a node 2205.

At step 2620, each of the nodes 2205 that selected the transaction may validate the transaction. Validating the transaction may include determining whether the transaction is legal or conforms to a pre-defined set of rules for that transaction, establishing user authenticity, and establishing transaction data integrity. At step 2625, if the transaction is successfully validated by a node 2205, the validated transaction is added to a block being constructed by that node 2205. As discussed above, since different nodes 2205 may choose to validate different transactions, different nodes 2205 may build or assemble a block comprising different validated transactions. Thus, the transaction associated with the first user 2110 transferring 10 units of cryptocurrency to the second user 2115 may be included in some blocks and not others.

At step 2635, the blockchain network 2130 may wait for a block to be published. Validated transactions may be added to the block being assembled by a node 2205 until it reaches a minimum size specified by the blockchain. If the blockchain network 2130 utilizes a proof of work consensus model, then the nodes 2205 may compete for the right to add their respective blocks to the blockchain by solving a complex mathematical puzzle. The node 2205 that solves its puzzle first wins the right to publish its block. As compensation, the winning node may be awarded a transaction fee associated with the transaction (e.g., from the wallet of the first user 2110). Alternatively, or in addition, the winning node may be awarded compensation as an amount of cryptocurrency added to an account associated with the winning node from the blockchain network (e.g., “new” units of cryptocurrency entering circulation). This latter method of compensation and releasing new units of cryptocurrency into circulation is sometimes referred to as “mining.” At step 2640, if a block has not been published, then the process 2600 returns to step 2635 and waits for a block to be published. However, at step 2640, if a block has been published, then the process 2600 proceeds to step 2645.

At step 2645, the published block is broadcast to the blockchain network 2130 for validation. At step 2650, if the block is validated by a majority of the nodes 2205, then at step 2655, the validated block is added to the blockchain 2130. However, at step 2650, if the block is not validated by a majority of the nodes 2205, then the process 2600 proceeds to step 2675. At step 2675, the block is discarded and the transactions in the discarded block are returned back to the queue. The transactions in the queue may be selected by one or more nodes 2205 for the next block. The node 2205 that built the discarded block may build a new next block.

At step 2660, if the transaction was added to the blockchain 2130, the server 2150 may wait to receive a minimum number of blockchain confirmations for the transaction. At step 2665, if the minimum number of confirmations for the transaction have not been received, then the process may return to step 2660. However, if at step 2665, the minimum number of confirmations have been received, then the process proceeds to step 2670. At step 2670, the transaction may be executed and assets from the first user 2110 may be transferred to the second user 2115. For example, the 10 units of cryptocurrency owned by the first user 2110 may be transferred from a financial account of the first user 2110 to a financial account of the second user 2115 after the transaction receives at least three confirmations.

Smart Contracts

A smart contract is an agreement that is stored in a blockchain and automatically executed when the agreement's predetermined terms and conditions are met. The terms and conditions of the agreement may be visible to other users of the blockchain. When the pre-defined rules are satisfied, then the relevant code is automatically executed. The agreement may be written as a script using a programming language such as Java, C++, JavaScript, VBScript, PHP, Perl, Python, Ruby, ASP, Tcl, etc. The script may be uploaded to the blockchain as a transaction on the blockchain.

As an example, the first user 2110 (also referred to as tenant 2110) may rent an apartment from the second user 2115 (also referred to as landlord 2115). A smart contract may be utilized between the tenant 2110 and the landlord 2115 for payment of the rent. The smart contract may indicate that the tenant 2110 agrees to pay next month's rent of $1000 by the 28th of the current month. The agreement may also indicate that if the tenant 2110 pays the rent, then the landlord 2115 provides the tenant 2110 with an electronic receipt and a digital entry key to the apartment. The agreement may also indicate that if the tenant 2110 pays the rent by the 28th of the current month, then on the last day of the current month, both the entry key and the rent are released respectively to the tenant 2110 and the landlord 2115.

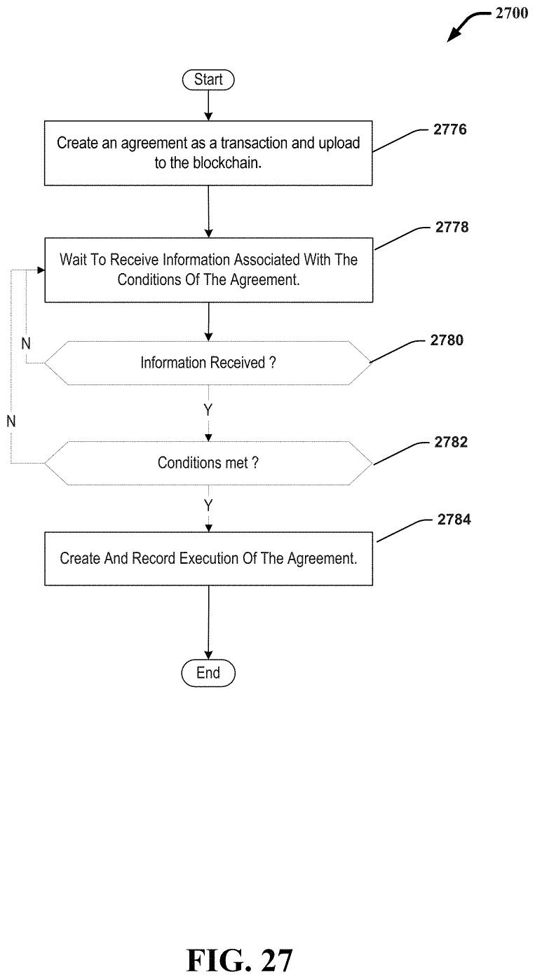

FIG. 27 is a flow diagram showing steps of an example method 2700 for performing a smart contract transaction between entities, such as the tenant 2110 and the landlord 2115. The steps of the method 2700 may be performed by any of the computing devices shown in FIG. 21. Alternatively or additionally, some or all of the steps of the method 270 may be performed by one or more other computing devices. Steps of the method 2700 may be modified, omitted, and/or performed in other orders, and/or other steps added.

At step 2776, the agreement or smart contract between the tenant 2110 and the landlord 2115 may be created and then submitted to the blockchain network 1030a as a transaction. The transaction may be added to a block that is mined by the nodes 2205 of the blockchain network 2130, the block comprising the transaction may be validated by the blockchain network 2130 and then recorded in the blockchain 2220 (as shown in steps 2610-2655 in FIG. 16). The agreement associated with the transaction may be given a unique address for identification.

At step 2778, the process 2700 waits to receive information regarding the conditions relevant for the agreement. For example, the process 2700 may wait to receive notification that $1000 was sent from a blockchain address associated with the tenant 1010 and was received at a blockchain address associated with the landlord 2115 by the 28th of the current month. At step 2180, if such a notification is not received, then the process 2700 returns to step 2778. However, if at step 2780, a notification is received, then the process 2700 proceeds to step 2782.

At step 2782, based on determining that the received notification satisfies the conditions needed to trigger execution of the various terms of the smart contract, the process 2700 proceeds to step 2784. However, at step 2782, if it is determined that the received notification does not satisfy the conditions needed to trigger execution of the smart contract, then the process 2700 returns to step 2778. At step 2784, the process 2700 creates a transaction associated with execution of the smart contract. For example, the transaction may include information of the payment received, the date the payment was received, an identification of the tenant 2110 and an identification of the landlord 2115. The transaction may be broadcast to the blockchain network 2130 and recorded in the blockchain 2220 (as shown in steps 2610-2655 of the process 2600 of FIG. 26). If the transaction is successfully recorded in the blockchain 2220, the transaction may be executed. For example, if the payment was received on the 28th, then an electronic receipt may be generated and sent to the tenant 2110. However, on the last day of the current month, both the digital entry key and the rent are released respectively to the tenant 2110 and the landlord 2115.

Smart contracts may execute based on data received from entities that are not on the blockchain or off-chain resources. For example, a smart contract may be programmed to execute if a temperature reading from a smart sensor or IoT sensor falls below 10 degrees. Smart contracts are unable to pull data from off-chain resources. Instead, such data needs to be pushed to the smart contract. Additionally, even slight variations in data may be problematic since the smart contract is replicated across multiple nodes of the network. For example, a first node may receive a temperature reading of 9.8 degrees and a second node may receive a temperature reading of 10 degrees. Since validation of a transaction is based on consensus across nodes, even small variations in the received data may result in a condition of the smart contract to be evaluated as being not satisfied. Third party services may be utilized to retrieve off-chain resource information and push this to the blockchain. These third-party services may be referred to as oracles. Oracles may be software applications, such as a big data application, or hardware, such as an IoT or smart device. For example, an oracle service may evaluate received temperature readings beforehand to determine if the readings are below 10 degrees and then push this information to the smart contract. However, utilizing oracles may introduce another possible point of failure into the overall process. Oracles may experience errors, push incorrect information or may even go out of business.

Since blockchains are immutable, amending or updating a smart contract that resides in a blockchain may be challenging and thus, more expensive and/or more restrictive than with text-based contracts.

### VI— Avatar Non-Funigle Tokens and Applications Thereof

One or more embodiments described herein can include systems, computer-implemented methods, apparatus, and/or computer program products that facilitate generating NFTs for unique avatars and employing the NFTs to regulate usage thereof in association with various applications.

As described with reference to avatar systems 100, 200, 1300 and 1400 described above, in various embodiments, an avatar system can provide for creating a personalized avatar for a user that functions as a desired persona for the user, such as the user's personal fitness trainer or coach, therapist, program guide, friend, family member, virtual assistant and so on. Such avatars are referred to hereinafter as “role avatars” as distinguished from “user identity avatars” which correspond to digital representations of actual users, described in greater detail below. The behavior of the role avatar is designed to guide or assist the user in association with performing fitness activities and other activities associated with a particular role of the avatar (e.g., therapist, assistant, guide, etc.). For example, the role avatar's behavior can resemble that which a real trainer would exhibit to provide instruction, demonstration, motivation, praise, coaching, etc. In this regard, based on monitoring activity of the user (e.g., using sensory feedback, image data captured of the user, context of the user, etc.) the role avatar can provide responses and instructions providing a mechanism to correct a deviation to a program, routine, or activity, an action taken by the avatar to deter or prevent the user from deviating from the program, etc., a tactic to motivate the user to adhere to the program, etc., a tactic to praise the user for adhering to the program, etc., or a tactic to criticize the user for not adhering to the program, etc. These reactions can involve visual and verbal actions which a real human may perform and can include but are not limited to changes in: motion or movement, speech, tone of voice, level of sound/loudness, facial expressions, body language, color, speed of movement, and range of motion. For example, the avatar can speak to the user and demonstrate body movement and provide facial expressions that a real human would in order to convey a reaction.

In some embodiments, the appearance, personality, behavior and other traits of a role avatar can be selected and/or tailored by the user (e.g., using avatar customization component 410 and/or avatar customization component 906). For example, the avatar customization component 410 can allow a user to customize the avatar by manipulating variables to create an avatar that reflects the needs and tastes of the user. In particular, using avatar customization component 410, a user can select the avatar's appearance, demographics (e.g., age, gender, ethnicity, language), voice, accent, dress, or other visual and/or audio characteristic of the avatar. In some implementations, the user can also choose a role avatar from a database of preconfigured avatar's having various appearances and personalities and further tailor or customize one or more characteristics of the avatar (e.g., appearance, clothing, voice, personality, etc.). In another aspect, avatar customization component 410 can automatically design an avatar to facilitate a user with a fitness routine or activity. According to this aspect, avatar customization component 410 can select the avatar's appearance, demographics, voice and personality based on one or more of: the fitness routine or activity selected for performance, profile information for the user regarding the user's preferences, the user's demographics, and the user's performance history with respect to monitored fitness routines or activities. For example, based on analysis of the user's preferences and demographics, avatar customization component 410 can determine or infer what type of avatar in terms of appearance and personality would best facilitate/motivate the user in association with performance of a selected fitness activity or routine (e.g., based on data relating avatar appearance and character traits to various aspects of user profile information).

In addition, the behavior and responses of a role avatar in association with responding to the user and guiding the user relative to a particular role of the avatar (e.g., fitness coach, therapist, program guide, etc.) can be tailored by the system based on preferences of the user, user demographics, language, location, context, monitored user activity and mood, and adapted and tailored over time based on learned user behavior and preferences (e.g., using machine learning and artificial intelligence). In this regard, role avatar responses to adherence or deviation from a routine, program, task or action (e.g., based on comparison to reference metrics or personally calibrated reference metrics) can be tailored based on user preferences, demographics, mood, language, location, or context. For example, with respect to an avatar that functions as a user's fitness trainer, an individual may provide preferences that note what type of exercises the user likes and doesn't like to perform or what type of coaching motivates the user (e.g., soft encouraging technique over a more pushy or demeaning approach). A response determined for the avatar to perform can then be tailored to account for the user's preferences. For instance, when the user is not achieving a particular exercise included in the fitness routine, the avatar can provide assurances that the user is “doing his best and that is all that matters,” as opposed to harping at the user and pushing the user “to get it right or get ten more minutes of abdominal work at the end of the session.” In another example, an avatar that is configured to provide instruction to a child can use different language and instructional techniques than an avatar configured to provide instruction to an adult. In yet another example, an avatar that is configured to provide guidance to a user throughout the day to adhere to a diet plan can suggest food options based on the user's context including the user's location, schedule and time of day.

Moreover, responses determined for manifestation by a role avatar (based on received user physical and physiological activity data) can be tailored based on learned user behavior. For example, historical data regarding past reactions/responses performed by the user to avatar responses in association with same or similar routines, tasks or actions can be collected and analyzed using various machine learning techniques to determine what types of avatar responses work and don't work for the user. In another example, traits, habits and abilities of the user can be learned over time and employed to tailor avatar responses (e.g., verbal responses, words/expressions states, tone of voice, volume, facial expressions, gestures, recommended actions, etc.). For instance, when suggesting a new exercise to transition to when a user is not achieving a previously suggested exercise, the avatar can select an exercise that the user is known to perform well to boost the user's spirit. In another example, when a user is known to stop at a fast-food restaurant on Elm street when experiencing high stress levels on the way home from work, an avatar designed to help the user adhere to a diet program can warn the user that the user is likely to perform this mistake and suggest an alternate route and food option for the user.

In this regard, the avatar systems described above can result in the generation of unique personalized “role” avatars with appearances and behaviors selected by the users and/or tailored to the user based on the user's preferences and activities over time. In one or more additional embodiments, these role avatars (e.g., providing a role as a coach, trainer, assistant, guide, etc.) can be regarded as intellectual property of their corresponding users and/or the avatar system. In this regard, the visual appearance of the avatar (e.g., computer code defining the visual appearance and/or and image data defining two-dimensional and three-dimensional image features thereof), the voice of the avatar, as well as code controlling the behavior and responses of the avatar e.g., algorithms, models, machine learning models and corresponding parameters as adapted/tailored to the user) can be regarded by the avatar system as intellectual property of the corresponding user and/or the avatar system. With these embodiments, the avatar system can associate the role avatar and its corresponding data that defines and controls the role avatar with the user identity associated with its creation and development and provide the user with authorization to control its usage thereof by other entities (e.g., other systems, other users, and so on).

In various embodiments, the avatar system can mint and/or authorize the user associated therewith to mint the role avatar as an NFT on the blockchain 1704. Once minted on the blockchain, the avatar system and/or the user can further control permissions regarding usage of the role avatar NFT by other users and systems. For example, the avatar system and/or the user can sell the role avatar NFT, license usage of the role avatar NFT by other users, gift usage of the role avatar NFT by other users and system, regulate usage of the role avatar NFT by other users and systems and so on, using permissions and agreements defined on the blockchain 1704. In some embodiments, the user and/or the avatar system can perform transactions for the usage of such role avatar NFTs in exchange for fees (e.g., cryptocurrency or another digital asset) which can be deposited into digital wallets of the avatar system and/or the users. Transactions involving the usage of the role avatar NFT (e.g., selling, licensing, gifting, granting usage thereof for specific usage scenarios) can be recorded on the blockchain 1704. In some implementations, these transactions can be defined and controlled automatically using smart contracts recorded on the blockchain 1704.

In addition, as described with reference to avatar systems 1300 and 1400 described above, in one or more embodiments, the avatar system can create personalized avatars corresponding to representations of users themselves based on image data captured of themselves. For example, the avatar visualization platform 1302 can generate a visual representation or replica (e.g., a user identity avatar) of a user based on received appearance information for the user (e.g., image data) and known health information for the user (including physical characteristics of the user and physiological characteristics of the user). As the user's appearance changes over the course of performance of a health and fitness program, the system can also adapt the appearance of the avatar to provide a mirrored visualization of the changes in the user.

In one or more additional embodiments, the avatar system can further develop a user identity avatar to reflect other characteristics of the user in addition to their appearance. For example, the system can model the user's behavior, personality, physical fitness capabilities, movements, gait, gestures, facial expressions, voice, actions, responses to stimuli and so on, in a manger that creates an intelligent model of the user in an avatar form. To facilitate this end, the system can employ the various input data described above that can be captured and monitored for a user via one or more sensors 104, cameras and/or one or more IFDs 118 in association with monitoring user activity relative to performance of fitness activities and other programs. For example, the system can learn and model the user's physical movements in association with performance of different fitness activities and physical abilities/capabilities and associated performance metrics (e.g., strength, range of motion, flexibility, physiological characteristics relative to different activities and contexts, etc.) based on data collected for the user over time. The system can further impart these physical and physiological characteristics onto the user identity avatar. The system can also learn and model the user's gestures based on motion and image data captured of the user. The system can also learn and model the user's voice based on audio samples captured from the user. The system can also learn and model the user's facial expressions and responses to stimuli based on image data captured for the user in association with monitoring the user activity over time relative to performance of fitness activities and other activities.

In accordance with these embodiments, the user identity avatars can be regarded as intellectual property of their corresponding users and/or the avatar system. In this regard, the visual appearance of the user identity avatar (e.g., computer code defining the visual appearance and/or and image data defining two-dimensional and three-dimensional image features thereof), the voice of the user identity avatar, as well as code controlling the behavior of the avatar e.g., algorithms, models, machine learning models and corresponding parameters as adapted/tailored to the user) can be regarded by the avatar system as intellectual property of the corresponding user and/or the avatar system. The avatar system can further associate the user identity avatar and its corresponding data that defines and controls the avatar with the user identity to which it corresponds and provide the user with authorization to control its usage thereof by other entities (e.g., other systems, other users, and so on) and/or in association with usage by the user/owner in other XR games and applications.

For example, in some embodiments, the avatar system can provide a gaming application wherein users can participate in fitness activities (and other activities) with other users' identity avatars in an AR and/or VR environment. For example, the gaming activity could involve competing against one or more other users' avatars in a fitness competition, a sports competition, a boxing match, a golf tournament or the like. In another example, the gaming activity could involve simply performing a fitness routine or group fitness class with avatar versions of one or more other users. In another example, the gaming activity could simply involve meeting with and conversing with the avatar version of another user. In accordance with these embodiments, a first user can perform the physical activity (or another activity) while being presented (e.g., as an AR object and/or a VR object) with the avatar version of a second user that has an appearance that resembles the actual appearance of the other second user. In addition, the behavior, motions, facial expressions, voice, physical abilities, verbal responses, physical responses and so on of the avatar of the second user in association with performing the physical activity (or the other activity) can reflect the actual behavior, motions, facial expressions, voice, physical abilities and responses, etc. of the actual second user identity to which its modeled after. For example, a first user could box the avatar version of a famous boxer or the avatar version of their friend. In another example, a first user could meet and converse with the avatar version of a celebrity, friend, or distant family member. In another example, a first user could go for a walk or run with, dance with, cook with, etc., the avatar version of a celebrity, friend, or family member presented to the first user via an AR device worn by the first user (e.g., AR googles, AR glasses, AR contact lenses etc.). In another example, a first user could play a team sport game (e.g., football, soccer, basketball, etc.) with avatar versions of other users presented to the first user as AR objects projected onto the sport field/environment. The other users could include friends, professional athletes, or essentially any person who has an avatar version of themselves modeled and created by the avatar system.

Still in other embodiments, a user can employ the avatar version of themselves in other XR games and environments provided by third-party systems. For example, a user can play other XR games (e.g., VR and/or AR games) provided by other systems using their avatar identity model generated by the avatar system. In some implementations of these embodiments, various gaming events and activities can grant permissions and authorizations to user based on usage of their avatar identity and verification that the avatar identity corresponds to the user's actual identity, appearance and physical capabilities.

The avatar system can also mint and/or authorize the user associated therewith to mint their identity avatar as an NFT on the blockchain 1704. Once minted on the blockchain, the avatar system and/or the user can further control permissions regarding usage of their identity avatar NFT by the user as well as other users and systems. For example, the avatar system and/or the user can restrict usage of the avatar identity to only the actual person corresponding to the avatar identity in other XR environments. In some implementations, the avatar system and/or the user can sell their identity avatar NFT, license usage of their avatar NFT by other users (e.g., in association the gamification aspect discussed above), gift usage of their identity avatar NFT by other users and system, regulate usage of the identity avatar NFT by other users and systems and so on, using permissions and agreements defined on the blockchain 1704. In some embodiments, the user and/or the avatar system can perform transactions for the usage of identity avatar NFTs in exchange for fees (e.g., cryptocurrency or another digital asset) which can be deposited into digital wallets of the avatar system and/or the users. Transactions involving the usage of the identity avatar NFTs (e.g., selling, licensing, gifting, granting usage thereof for specific usage scenarios) can be recorded on the blockchain 1704. In some implementations, these transactions can be defined and controlled automatically using smart contracts recorded on the blockchain 1704.

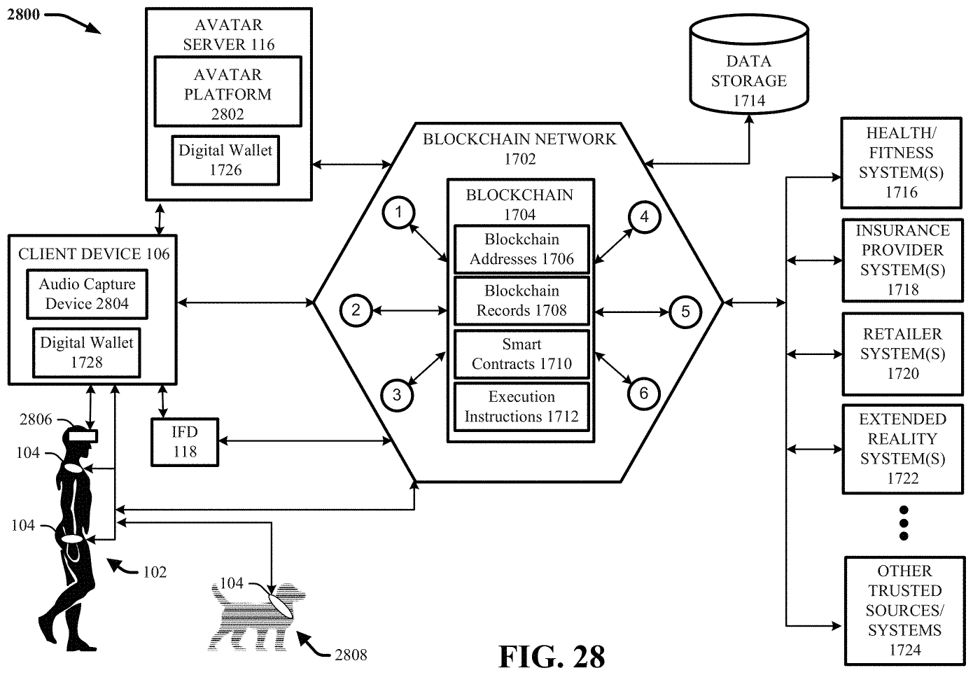

Referring to the drawings, FIG. 28 illustrates a high-level block diagram of an example, non-limiting system 2800 that can facilitate generating avatar NFTs and applications related to usage thereof in accordance with one or more embodiments described herein. System 2800 is similar to system 1700 with the addition of avatar platform 2802 to the avatar server 116, an audio capture device 2804 to the client device 106, a XR device 2806 worn by the user 2806, and an animal 2808 (depicted as a dog but can correspond to any type of animal) coupled to (e.g., physically worn by and/or implanted therein) one or more sensors 104. Repetitive description of like elements employed in respective embodiments is omitted for sake of brevity

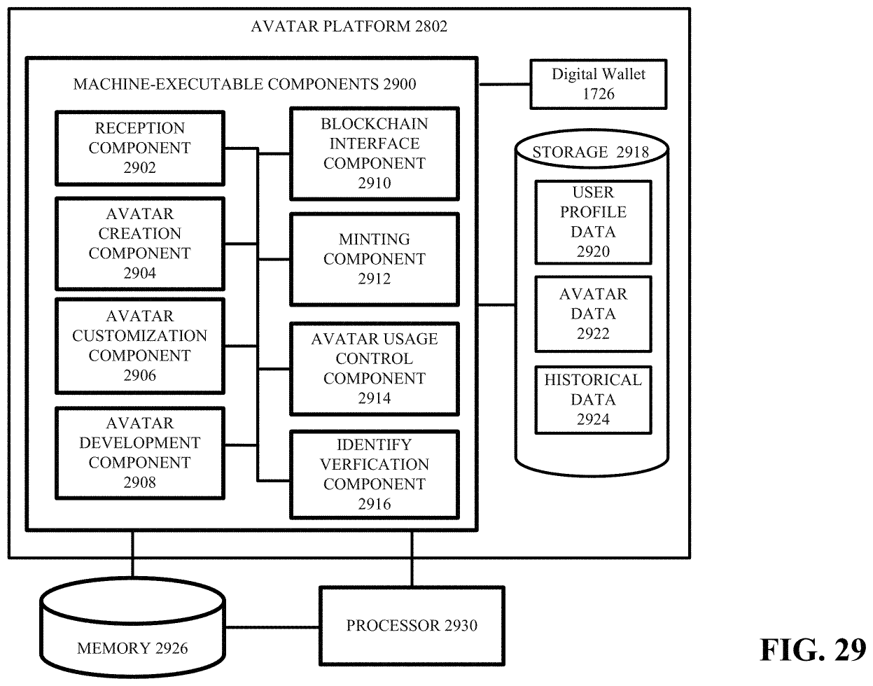

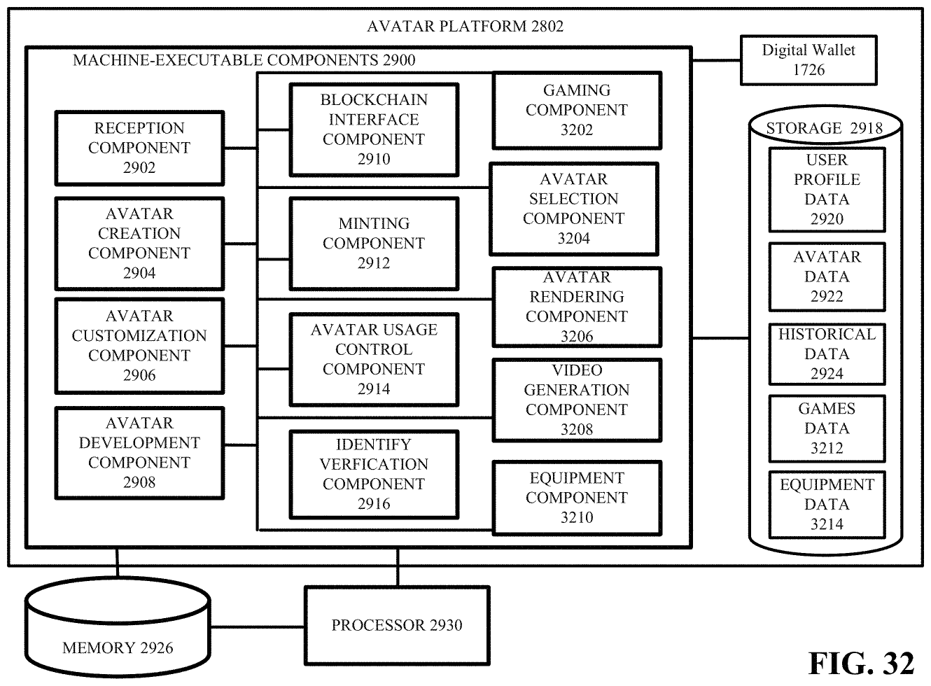

Avatar platform 2802 can provide various additional features and functionalities relative to previously described avatar platforms (e.g., avatar platform 114, avatar guidance platform 202, and avatar visualization platforms 1302 and 1402) that facilitate generating avatar NFTs and applications related to usage thereof. These additional features and functionalities are described with reference to FIGS. 29 and 32. Avatar platform 2802 can further include any of the features and functionalities of avatar platform 114, avatar guidance platform 202, and avatar visualization platforms 1302 and 1402. The audio capture device 2802 can correspond to any suitable audio capture device configured to receive, record and optionally interpret (e.g., speech to text) sound, speech and audio provided by users (e.g., user 102) and animals (e.g., animal 2808). In various embodiments, the recorded speech, sound and other audio can be used by the avatar server 116 in association creating avatar versions of the user that speak and/or sound like the users.

The XR device 2806 can correspond to any suitable XR device designed for wear by a user 102 as googles, glasses, a head-up displays, head mounted devices, holographic devices, smart contact lenses or the like that can provide XR renderings and related applications. The term extended reality or XR is used herein to collectively refer to AR, VR or combinations thereof (also referred to as mixed reality). AR or mixed reality enabled devices overlay digital content on top of a real-world view, thus, enhancing a user's experience of the real world. The overlay content may be 2D and/or 3D models generated based on image scanning real world objects. As applied to various embodiments of the disclosed subject matter, the overlay content can include 2D and/or 3D avatars, including role avatars, user identity avatars, and animal avatars. These digital representations can comprise 2D and/or 3D models generated by the avatar platform 2802 based on image data captured of users and/or animals (e.g., via one or cameras of the client device 106, the IFD 118, or another image scanning device).

VR devices or headsets correspond to entirely immersive environment wherein the user is presented with a computer-generated simulation of a three-dimensional image or environment that can be interacted with in a seemingly real or physical way by a person using special electronic equipment, such as a helmet with a screen inside or gloves fitted with sensors. The field of view in a VR environment does not include a view of the real-world or real-world objects. In some embodiments, 2D and/or 3D avatars can be presented to users in a VR environment (e.g., provided by the avatar platform 2802 and/or other extended reality systems 1722), including role avatars, user identity avatars and animal avatars (as discussed below). For example, the user can interact with the VR environment from the perspective of a person immersed in the environment in a shared experience with other players or people being located within the immersive environment. With these embodiments, the respective players or users can be embodied as virtual avatars and interact with one another as avatars. The respective players can choose their avatar to represent themselves to others, which in various embodiments can include their own identity avatar generated by the avatar platform 2802.

FIG. 29 presents an example illustration of avatar platform 2802. The avatar platform 2802 comprises various machine-executable components 2900 that can facilitate generating avatar NFTs and controlling usage thereof using blockchain network 1702, in accordance with one or more embodiments described herein. The machine-executable components 1800 include reception component 2902, avatar creation component 2904, avatar customization component 2906, avatar development component 2908, blockchain interface component 2910, minting component 2912, avatar usage control component 2914, and identity verification component 2916. The avatar platform 2802 further includes digital wallet 1726, and storage 2818 which can include user profile data 2920, avatar data 2922 and historical data 2924. The machine-executable components 2900 can be stored in at least one computer-readable memory 2926 that is operably and/or operatively and/or communicatively connected/coupled to at least one processor 2930 (e.g., a computer processing unit, a microprocessor, etc.), which upon execution by the processor 2930 can cause the processor 2930 to perform the corresponding acts or operations of the components described below. In some embodiments, one or more of the machine-executable components 2900 can be associated with one or more devices of the blockchain network 1702. Additionally, or alternatively, one or more of the machine-executable components 2000) can be associated with the client device 106. Various different implementations and architectural configurations are envisioned.

With reference to FIG. 29 in view of FIG. 28, the reception component 2902 can receive a variety of inputs for users described herein that can be used to create and develop avatar NFTs for users. In this regard, the inputs can include any of the information captured via one or more sensors 104 worn by and/or attached to the user, image data (e.g., image data and/or video data) captured via one or more cameras (e.g., associated with the client device 106, the IFD 118 and/or the XR device 2806, any of the input information described with reference to FIG. 3 (e.g., profile information 302, raw physical data 304, context information 306), and the various health and fitness information described herein. In some embodiments, all or some of this information received for respective users can be associated with respective profiles for the users included in profile data 2920. The profile data can also include any of the user profile information previously described (e.g., profile data 222, and user profile data 1314). Additionally, or alternatively, the reception component 2902 can aggregate and track relevant health and fitness information, image data and context information for users in as historical data 2924.

The avatar creation component 2904 can create avatars for users, including role avatars, identity avatars and animal avatars (as described below). The avatar creation component 2904 can include any of the features and functionalities described with reference to visualization component 1318 and additional features and functionalities described herein. In this regard, as applied to user identity avatars, the avatar creation component 2904 can generate digital image representations of users based on image data captured thereof that visually correspond to the actual appearance of the respective users. For example, the digital representations can comprise two and/or three-dimensional image models of the respective users generated by the avatar creation component based on image scans of the users. Avatar representations (e.g., two and/or three-dimensional image models) for respective users can be associated with their profile data 2920 and/or in avatar data 2922. The avatar creation component 2904 can also receive initial facial expression data for users and impart facial expressions onto the avatar representations. For example, users can provide different facial expressions in image data in response to defined prompts (e.g., requesting different defined facial expressions) and stimuli. These facial expressions can be recorded and modeled to cause the avatar to perform the corresponding facial expressions as applicable in response to same or similar stimuli and/or learned stimuli for the respective users. The avatar creation component 2904 can also receive audio data captured for users to generate and apply sound and voice features to the avatar models. For example, the avatar creation component 2904 can receive audio samples for the respective users and model their voice characteristics with respect to tone, inflection, pace, accent, language and so on. In this regard, the avatar creation component 2904 can generate audio models for the respective user identity avatars that correspond to the actual voice characteristics of the users to which they correspond. The avatar creation component 2904 can also receive initial physical movement and motion data for the respective users to generate and impart initial physical movement characteristics onto the avatars. For example, users can perform various movements and gestures, movements (e.g., walking, jumping, dancing, siting, etc.) in response to prompts which can be captured via motion sensors worn by the user and/or determined based on image data/video captured of the user. The avatar creation component 2904 can impart these physical motions and gesture characteristics onto the avatar models. In some embodiments, the avatar creation component 2904 can also receive initial physical assessment data for a user regarding their physical fitness performance capabilities (e.g., strength, agility, range of motion, etc.) and incorporate these performance capabilities into the avatar model for the user. In this regard, the avatar creation component 2904 can create an identity avatar for a user that looks like, moves like and sounds like the corresponding user based on some initial data received for the user.

The avatar customization component 2906 can provide various interactive features and functionalities for users to provide input customizing or tailoring their identity avatars and/or role avatars. The avatar customization component 2906 can provide any of the features and functionalities described with reference to avatar customization component 410 and/or avatar customization component 906 and additional features and functionalities described herein. For example, the avatar customization component 2906 can provide tools that allow a user select and/or design the avatar clothing, hairstyle, skin color, make-up, and/or adjust the shape and appearance of their identity avatar, adjust the voice characteristics, the gesture and movement characteristics, and on. In some implementations as applied to identity avatars, the avatar customization component 2906 can restrict the degree of adjustment in appearance and/or other characteristics of the avatar (e.g., voice, gestures, movement, facial expressions, etc.) so that the avatar remains a realistic representation of themselves as a recognizable person when used in AR and VR applications in association with interacting with outer entities and users. In some embodiments, a user can adjust their initial identity avatar to any degree in association with converting the avatar into a role avatar. With these embodiments, a user can create a role avatar from their initial identity avatar generated based on image data captured of themselves and adjusting the appearance and/or other attributes of the avatar to a requisite amount. In other embodiments, users can create role avatars from a set of preconfigured avatar models provided in the avatar data 2922. In some embodiments, users can license, sell or gift their identity avatars to other users (or all users) for use as role avatars. In this regard, a user can make his or her initial representation or developed representation (as described below) identity avatar available for use by other users as role avatars (e.g., virtual coaches, therapists, trainers, friends, etc.). For instance, a first user may user the identity avatar of the real-life friend, family member, trainer etc., to embody corresponding role avatars for the first user in association with usage with the avatar systems described herein and/or in association with usage in one or more externa XR systems.

The avatar development component 2908 can further develop and tailor the behavior, capabilities, responses and intelligence of role avatars, identity avatars and animal avatars based on additional data learned and captured for respective users over time using machine learning and artificial intelligence. For example, as applied to role avatars, the avatar development component 2908 can tailor the behavior and responses of a user's role avatar such as their virtual coach to account for monitored user data captured for the user (e.g., sensory data, voice data, user/avatar interaction data, etc.) in association with performing various fitness activities, as described with reference to system 100 and system 200. Similarly, the avatar development component 2908 can learn the behavior, physical fitness capabilities, responses to stimuli and different context and events, habits, preferences, etc. of a user in association with monitoring the user during fitness activities and other activities. The avatar development component 2908 can further impart these learned features and capabilities onto the user's identity avatar. In this regard, over time, a user's identity avatar will look act and behave more and more like the actual user in real-life. In some embodiments, the avatar development component 2908 can also employ user provided and/or learned user profile information regarding preferences of the user and/or describing the user's personality traits and behavioral traits in association with developing the user's identity avatar such that the behavior and responses (e.g., verbal and physical) of the users' identity avatar reflect those of the real-user to which its modeled.

In this regard, the avatar representations, including role avatars, identity avatars and animal avatars can respectively comprise a unique set of image data (e.g., 2D and/or 3D image models that define the shape and visual appearance of the respective avatars). Each of the avatar representations can further be associated with unique audio files that encode and/or model the voice characteristics of the respective avatars, and unique sets of computer-executable code that control the behavior (e.g., physical and verbal response) of the avatars, the movement of the avatars, the physical fitness capabilities and actions of the avatars and so one. In some embodiments, some or all of the information controlling and defining the avatar behavior, movement, physical fitness capabilities and actions, responses to stimuli and other intelligence of the avatar can be modeled using one or more machine learning and/or artificial intelligence models generated by the avatar development component 2908. These models can include explicitly trained models (e.g., supervised ML models), implicitly trained models (unsupervised ML models), and combinations thereof. All information (e.g., models, image files, audio files, etc.) defining and controlling the appearance and behavior of avatars associated with respective users (e.g., identity avatars, role avatars and animal avatars) can be stored in avatar data 2922 and regularly and/or continuously updated based on new feedback received over time.

The blockchain interface component 2910 can provide a suitable application program interface (API) for connecting the avatar platform 2802 (and/or client devices 106) to the blockchain network 1702 and allowing the avatar platform 2802 and/or respective users (e.g., client devices 106) to access the blockchain network and read to and/or write from the blockchain in accordance with defined permissions (e.g., defined by the blockchain execution instructions 1712).

In various embodiments, the avatar platform can mint (e.g., via minting component 2912, minting component 1816, or the like) and/or authorize the user associated therewith to mint one or more of their role avatars as an NFT on the blockchain 1704. The minting component 2912 can provide the same or similar minting functionalities described with reference to minting component 1816). In some embodiments, all or portions of the data (i.e., code, models, files, etc.) defining a role avatar can be minted as an NFT on the blockchain 1704. For example, in some implementations, only the image model (e.g., a 2D and/or 3D image model) of the avatar may be minted as an NFT. In other implementations, additional data defining and controlling the voice, behavior, gestures, facial expressions, physical fitness capabilities, etc., of a role avatar can be minted as NFT on the blockchain 1704. In some embodiments, the avatar platform 2802 can establish expressed agreements with users that requires the users to assign intellectual property rights of role avatar NFTs to the avatar system provider (e.g., an entity or company providing the avatar platform). With these embodiments, the minting component 2912 can mint role avatars in association with assigning ownership to the avatar provider and in turn provide ownership of the role avatar NFTs to the avatar provider (e.g., minting to a blockchain address associated with the avatar provider, accessible via digital wallet 1726, and issuing the NFTs to the avatar provider). In other embodiments, the avatar platform can assign ownership of role avatars to their corresponding users and allow users to mint their role avatars as NFTs in their own name (e.g., assigned to their own identities and blockchain addresses). Still in other embodiments, the avatar platform 2802 can regulate ownership of role avatar NFTs as jointly owned between the avatar provider and the users respectively associated therewith.

In association with minting role avatars as NFTs on the blockchain 1704, the avatar usage control component 2914 can provide for defining and controlling permissions regarding usage of the role avatar NFT by other users and systems. For example, the avatar system and/or the user can sell the role avatar NFT, license usage of the role avatar NFT by other users (e.g., for a fee), gift usage of the role avatar NFT by other users and system, regulate usage of the role avatar NFT by other users and systems and so on, using permissions and agreements defined on the blockchain 1704 and/or the avatar data 2922. The avatar usage control component 2914 can provide for defining these permissions and agreements, facilitate recording them on the blockchain (e.g., where applicable) and facilitate initiating and/or executing transactions regarding usage of the role avatar NFTs. In some embodiments, the user and/or the avatar platform can perform transactions for the usage of such role avatar NFTs in exchange for fees (e.g., cryptocurrency or another digital asset) which can be deposited into digital wallets of the avatar system and/or the users depending on the ownership rights associated therewith. Transactions involving the usage of the role avatar NFTs (e.g., selling, licensing, gifting, granting usage thereof for specific usage scenarios) can be recorded on the blockchain 1704. In some implementations, these transactions can be defined and controlled automatically using smart contracts (e.g., one or more of smart contracts 1710 recorded on the blockchain 1704 and established using the avatar usage control component 2914.

The avatar system can also mint and/or authorize the user associated therewith to mint (e.g., using minting component 2912 and/or minting component 1816) their identity avatar as an NFT on the blockchain 1704. As with the role avatars, all or portions of the data (i.e., code, models, files, etc.) defining an identity avatar can be minted as an NFT on the blockchain 1704. As with role avatars, the avatar platform 2802 can consider identity avatars as intellectual property of the respective users to which they are modeled, the avatar platform provider and/or a joint ownership. In this regard, depending on the ownership right agreements established between the avatar provider and the respective users, the identity avatar NFTs can be minted and issued to either their users, the avatar provider, or a combination thereof.

In association with minting identity avatars as NFTs on the blockchain 1704, the avatar usage control component 2914 can provide for defining and controlling permissions regarding usage of the identity avatar NFTs by other users and systems. In some embodiments, the avatar usage control component 2914 can restrict usage of an identity avatar NFT to only the actual person corresponding to the identity avatar in other XR environments using permissions and agreements defined on the blockchain 1704 and/or the avatar data 2922. The identify verification component 2916 can also perform an identity verification process that links the actual person represented in an identity avatar to the identity avatar and verifies the person represented in an identify avatar corresponds to the actual person. The identity verification component 2916 can further facilitate verifying the identify of an entity using or requesting to use an identity avatar in external (e.g., outside the avatar platform, such as one or more external XR systems 1722) XR environment corresponds to the user/person after which the identity avatar was modeled.

Additionally, or alternatively, the avatar system and/or the user can sell their identity avatar NFT, license usage of their avatar NFT by other users (e.g., in association the gamification aspect discussed above), gift usage of their identity avatar NFT by other users and system, regulate usage of the identity avatar NFT by other users and systems and so on, using permissions and agreements defined on the blockchain 1704 and/or the avatar data 1922. In some embodiments, the user and/or the avatar system can perform transactions for the usage of identity avatar NFTs in exchange for fees (e.g., cryptocurrency or another digital asset) which can be deposited into digital wallets of the avatar system and/or the users. Transactions involving the usage of the identity avatar NFTs (e.g., selling, licensing, gifting, granting usage thereof for specific usage scenarios) can be recorded on the blockchain 1704. In some implementations, these transactions can be defined and controlled automatically using smart contracts recorded on the blockchain 1704.

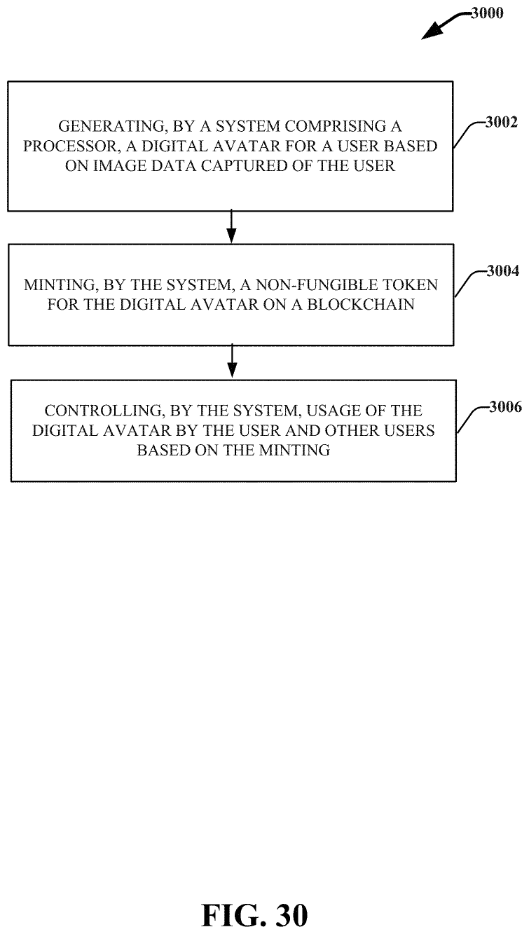

FIG. 30 presents an example computer-implemented method 3000 for generating avatar NFTs in accordance with one or more embodiments described herein. Method 3000 corresponds to an example computer implemented method that can be performed via system 2800 using one or more computer executable components 2900. Repetitive description of like elements employed in respective embodiments is omitted for sake of brevity.

At 3002, method 3000 comprises generating, by a system comprising a processor (e.g., system 2800), a digital avatar for a user based on image data captured of the user (e.g., an identity avatar generated using avatar creation component 2904 and/or avatar development component 2908). At 3004, method 300 comprises minting, by the system, an NFT for the digital avatar on a blockchain (e.g., on blockchain 1704 using minting component 2912). At 3006, method 3000 comprises controlling, by the system, usage of the digital avatar by the user and other users based on the minting (e.g., via avatar usage control component 2914). In various implementations, the controlling can comprise controlling usage of the digital avatar by a user identity in association with usage of the digital avatar in one or more virtual reality, augmented reality, or extended reality environments (e.g., provided by the avatar server 116 and/or one or more external XR systems 1722) based on verification that the user identity corresponds to the user and verification that the NFT is owned by the user via the blockchain (e.g., via identity verification component 2916). In other implementations, the controlling can comprise controlling usage of the digital avatar by a user identity (e.g., other than the user) in association with usage of the digital avatar in one or more virtual reality, augmented reality, or extended reality environments (e.g., provided by the avatar server 116 and/or one or more external XR systems 1722) based on verification that the user identity is authorized to use the digital avatar based on one or more smart contracts or usage permissions for the NFT defined on the blockchain (e.g., via identity verification component 2916).

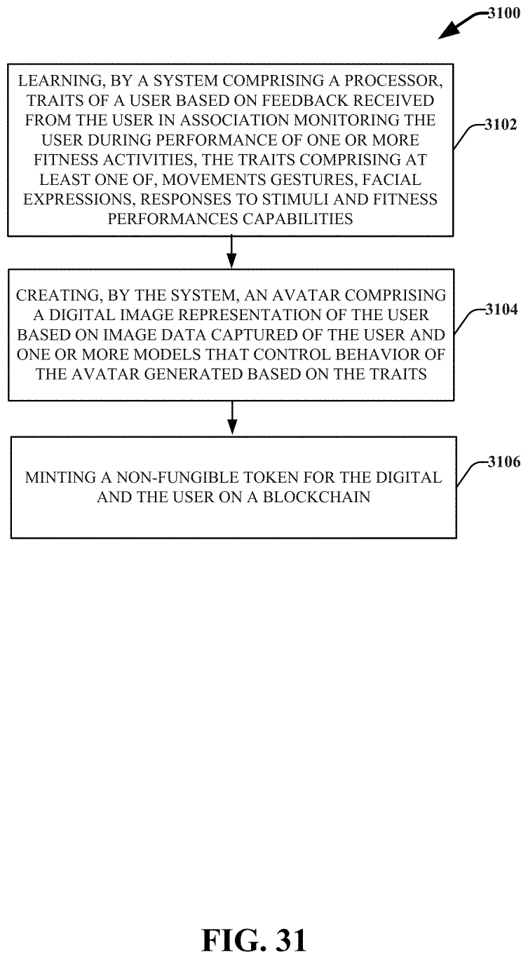

FIG. 31 presents another example computer-implemented method 3100 for generating avatar NFTs in accordance with one or more embodiments described herein. Method 3100 corresponds to another example computer implemented method that can be performed via system 2800 using one or more computer executable components 2900. Repetitive description of like elements employed in respective embodiments is omitted for sake of brevity.

At 3102, method 3100 comprises learning, by a system comprising a processor (e.g., system 2800 using avatar development component 2908 and one or more machine learning processes), traits of a user based on feedback received from the user in association monitoring the user during performance of one or more fitness activities, the traits comprising at least one of, movements, gestures, facial expressions, responses to stimuli and fitness performances capabilities. At 3104, method 3100 comprises creating, by the system, an avatar comprising a digital image representation of the user based on image data captured of the user and one or more models that control behavior of the avatar generated based on the traits (e.g., via avatar creation component 2904 and avatar development component 2908). At 3106, method 3100 further comprises minting an NFT for the avatar and the user on a blockchain (e.g., blockchain 1704 via minting component 2912).

FIG. 32 presents another example embodiment of avatar platform 2802 that facilitates extended reality applications using avatars. In accordance with the embodiment shown in FIG. 32, the machine-executable components 2900 can further include gaming component 3202, avatar selections component 3204, avatar rendering component 3206, video generation component 3208 and equipment component 3210. The storage 2918 can further include games data 3212 and equipment data 3214.

As described above, in various embodiments, the avatar platform 2802 can associate user identity avatars and the corresponding data that defines and controls the respective avatars with the users to which they are modeled after. The avatar usage control component 2914 can further provided mechanisms for the avatar service provider and/or the respective users to control usage thereof by other entities (e.g., other systems, other users, and so on) and/or in association with usage in other XR games and applications. In some embodiments, the mechanism can be based on minting ownership rights for the identity avatars on the blockchain as NFTs and controlling their usage via agreements, permissions and/or smart contacts defined and/or executed on the blockchain 1704.

In some implementations of these embodiments, the gaming component 3202 can execute and/or run the various XR games and applications as stored in games data 3212 and the avatar rendering component 3206 can render the avatars included in the game in accordance with their respective data avatar data 2922 as provided in storage 2818 that defines and controls the appearance, behavior and responses of the avatars (e.g., using their respective models and learned and/or defined attributes, such as gestures, responses, motions, fitness abilities, performance metrics, voice models, etc.). Depending on the type of the game, the avatar rendering component 3206 can render the avatars as AR objects on an XR device 2806 worn by the user 2802, and/or the client device 106 in an AR environment, and/or as VR objects on an XR device 2806 worn by the user 2802, and/or the client device 106 in an VR environment. In other embodiments, the avatar data 2922 can be recorded on the blockchain and accessed and executed in association with accessing and/or retrieving the avatar data on the blockchain 1704. In other embodiments, the XR games and applications can be provided by external XR systems 1722.

The avatar selection component 3204 can provide for selecting avatars (e.g., other user identity avatars, animal avatars and role avatars), to use as players in an XR game in accordance with the usage permissions defined therefore (e.g., licensing agreements, in exchange for a fee in the form of cryptocurrency or another digital asset, and so on). For example, the avatar selection component 3204 can provide the user with access to information identifying and describing other avatars available for usage to the user and identify any restrictions or permissions associated with the usage (e.g., free/openly without restriction, for a fee, under restricted permissions, etc.) in a particular game. The user can further select desired avatars for usage from the available list.

The XR games and applications can vary. For example, in some embodiments, the XR gaming applications (e.g., provided in games data 3212 and/or provided by one or more XR systems 1722) can include a game wherein users can participate in gaming activities (e.g., fitness activities and other activities) with other users' identity avatars in an AR and/or VR environment. For example, the fitness activity could involve competing against one or more other users' avatars in a fitness competition, a sports competition, a boxing match, a golf tournament or the like. In another example, the gaming activity could involve simply performing a fitness routine or group fitness class with avatar versions of one or more other users. In another example, the gaming activity could simply involve meeting with and interacting with the avatar version of another user. In accordance with these embodiments, a first user can perform the physical activity (or another activity) while being presented (e.g., as an AR object and/or a VR object) with the avatar version of a second user that has an appearance that resembles the actual appearance of the other second user. In addition, the behavior, motions, facial expressions, voice, physical abilities, verbal responses, physical responses and so on of the avatar of the second user in association with performing the physical activity (or the other activity) can reflect the actual behavior, motions, facial expressions, voice, physical abilities and responses, etc. of the actual second user identity to which its modeled after. For example, a first user could box the avatar version of a famous boxer or the avatar version of their friend. In another example, a first user could meet and interact with the avatar version of a celebrity, friend, or distant family member. In another example, a first user could go for a walk or run with, dance with, cook with, etc., the avatar version of a celebrity, friend, or family member presented to the first user via an AR device worn by the first user (e.g., AR googles, AR glasses, AR contact lenses etc.). In another example, a first user could play a team sport game (e.g., football, soccer, basketball, etc.) with avatar versions of other users presented to the first user as AR objects projected onto the sport field/environment (e.g., either a rea-world sports field/environment in AR applications or a virtual sports field/environment in VR applications). The other users could include friends, professional athletes, or essentially any person who has an avatar version of themselves modeled and created by the avatar platform 2802.

In some embodiments, the video generation component 3208 can record and generate videos of XR games involving avatars. For example, assuming a first player selects one or more other avatars (identity avatars of other users) to play a sports game with in AR or VR, the video generation component 3208 can generate a video from the perspective of the first player that includes the interactions between the first player and the one or more other avatars with the one or more other avatars appearing as AR and/or VR objects in the corresponding gaming environment (e.g., either a real-world environment, a digital VR environment, or a mixed reality environment. Additionally, or alternatively, the video generation component 2308 can generate a video from the perspective of an audience watching the sports game, replacing the first user with their identity avatar or another avatar whose behavior and responses mimic those of the first user. Still in other embodiments, the video generation component 3208 can generate video from the perspectives of any avatar player in the game. Users can further save their videos of their games (e.g., in association with their user profile data 2920), share their videos or clips therefrom on social media channels, and perform various other usages of their videos.

In some embodiments, videos generated by the video generation component 3208 can be considered intellectual property of the user playing the game (i.e., the first user in the example above) and/or the avatar platform service provider 2802. The avatar platform service provider can further mint and/or authorize the user associated therewith to mint the videos generated by the video generation component 3208 as NFTs on the blockchain 1704 (e.g., using minting component 2912). Once minted on the blockchain, the avatar system and/or the user can further control permissions regarding usage of the videos by other users and systems. For example, the avatar system and/or the user can sell the avatar video NFTs, license usage of the avatar video NFTs by other users, gift usage of the avatar video NFTs by other users and system, regulate usage of the avatar video NFTs by other users and systems and so on, using permissions and agreements defined on the blockchain 1704. In some embodiments, the user and/or the avatar system can perform transactions for the usage and/or ownership of avatar video NFTs in exchange for fees (e.g., cryptocurrency or another digital asset) which can be deposited into digital wallets of the avatar system and/or the users. Transactions involving avatar video NFTs (e.g., selling, licensing, gifting, granting usage thereof for specific usage scenarios) can be recorded on the blockchain 1704. In some implementations, these transactions can be defined and controlled automatically using smart contracts recorded on the blockchain 1704.

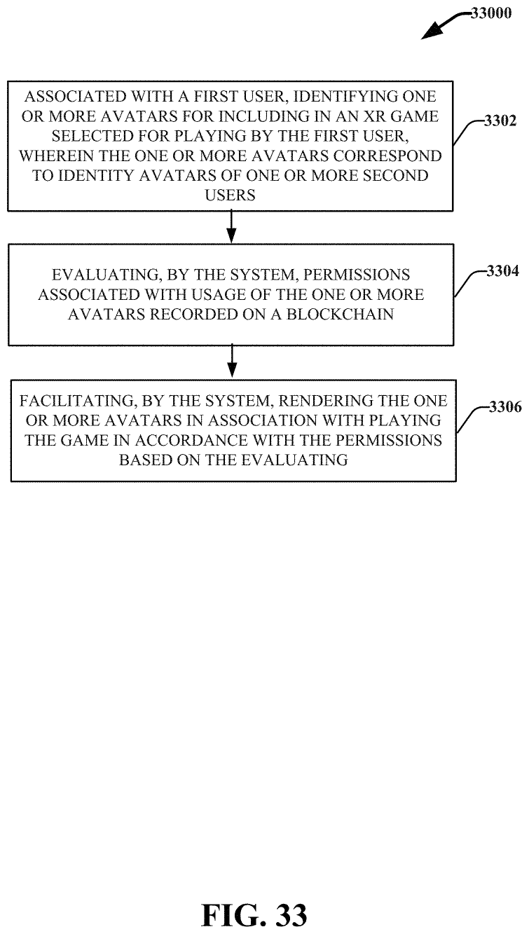

FIG. 33 presents an example computer-implemented method 3300 for employing other user's avatars in association with XR gaming applications, in accordance with one or more embodiments described herein. Method 3300 corresponds to an example computer implemented process that can be performed by the avatar platform 2802. Repetitive description of like elements employed in respective embodiments is omitted for sake of brevity.

In accordance with method 3300, at 3302, a system comprising a processor (e.g., system 2800) receives a selection associated with a first user, identifying one or more avatars for including in an XR game selected for playing by the first user (e.g., via avatar selection component 3204), wherein the one or more avatars correspond to identity avatars of one or more second users. At 3304, the system evaluates permissions associated with usage of the one or more avatars recorded on a blockchain (e.g., blockchain 1704, using avatar usage control component 2914). At 3306, the system facilitates rendering the one or more avatars in association with playing the game in accordance with the permissions based on the evaluating (e.g., via avatar rendering component 3206).

### VII—Fitness Equipment Extend Reality Applications

One or more embodiments are further directed to facilitating fitness equipment utilization using avatars rendered to users in AR and/or VR. With reference to FIGS. 28 and 32, in some embodiments, the equipment component 3210 can identify and recognize real-fitness equipment included in an environment (e.g., a public gym or the like and/or a private gym or the like) based on image data captured of the fitness equipment (e.g., via one or more cameras on the client device 106 and/or one or more cameras associated with the XR device 2806). For example, a user can view/scan an environment comprising a variety of different fitness equipment via an AR device worn by the user and capture image data of the fitness equipment. The fitness equipment can include a variety of different fitness equipment, such as machines (e.g., weight machines, cardio machines, etc.) and auxiliary equipment (e.g., free weights, barbells, stability balls, bands, etc.). The equipment component 3210 can further employ object recognition and image analysis techniques to match the equipment with known equipment defined in the equipment data 3214. Additionally, or alternatively, the equipment can be associated with a barcode or QR code which can be scanned via one or more cameras on the XR device 2806 and/or the user device 106 which links to information in the equipment data identifying the corresponding equipment.

In addition to information identifying different fitness equipment, the equipment data 3214 can also include usage models that comprise avatar models providing a demonstration of how to use the corresponding equipment. In this regard, a user can select a particular piece of fitness equipment and be provided with a visual demonstration (e.g., in AR and/or VR) of an avatar performing how to use the equipment. In some embodiments, the visual demonstration can be provided in AR with the avatar being presented to the user as a 2D or 3D object aligned relative to the real equipment in space on the user AR device. In this regard, the user can be presented with an avatar overlaid onto the actual equipment while demonstrating usage of the equipment. The avatar can also interact with other AR representations of the equipment where applicable in association with the demonstration (e.g., AR representations of the equipment moving in use by the avatar object and/or AR representations of the avatar using AR objects corresponding to the equipment, such as the avatar using free weights and performing an exercise therewith). In some embodiments, the particular avatar presented to the user and providing the demonstration can be selected by the user (e.g., via avatar selection component 3204) and correspond to their own user identity avatar, a role avatar, or another user's avatar.

In addition, the visual demonstration can be tailored to account for the physical size/shape of the user (e.g., height, weight, etc.), the fitness capabilities of the user and the fitness goals of the user (e.g., as provided in their profile data 2920). For example, the visual demonstration can adjust the settings of the equipment, the position of the user relative to the equipment, the weight used, the intensity used, and so on based on the needs, abilities and preferences of the user.

Still in other embodiments, with reference again to fitness module 230 and FIG. 4, the routing builder component 412 can automatically generate a fitness routine for the user based on the equipment available to the user in the scanned environment. For example, based on the equipment available in a scanned environment, the fitness capabilities of the user, the preferences of the user, the historical workout history of the user (e.g., what fitness activities the user performed yesterday, the day before, etc.), the current health state of the user, the current fatigue state of the user, and so one, the routine builder component 406 can automatically generate a fitness workout or routine for the user using some or all of the available equipment (e.g., using machine learning and artificial intelligence). The avatar system can further generate a visual demonstration of the routine and/or usage of the equipment in accordance with the generated routine that involves demonstration via an avatar (e.g., presented in AR and/or VR).

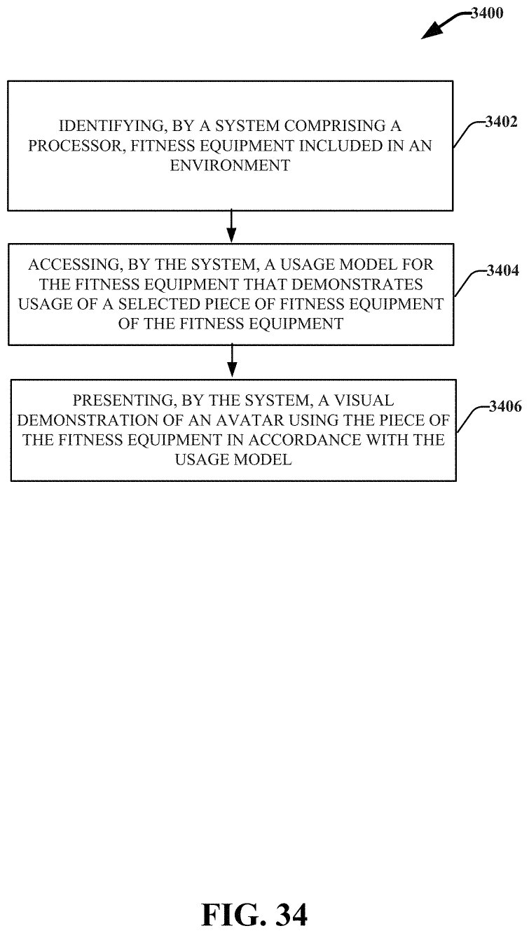

FIG. 34 illustrates an example computer-implemented method 3400 for demonstrating usage of equipment using an avatar, in accordance with one or more embodiments described herein. Method 3400 comprises identifying, by a system comprising a processor (e.g., system 2800), fitness equipment included in an environment (e.g., using equipment component 3210). Method 3400 further comprises accessing, by the system, a usage model for the fitness equipment that demonstrates usage of a selected piece of fitness equipment of the fitness equipment (e.g., provided in equipment data 3214). Method 3400 further comprises presenting, by the system, a visual demonstration of an avatar using the piece of the fitness equipment in accordance with the usage model (e.g., via equipment component 3210 and/or avatar rendering component 3210).

### VIII—Avatar Animals

The disclosed techniques for generating and developing user identity avatars can also be extended to animals, such as pets, zoo animals, racehorses, race dogs, and essentially any animal capable of being visually captured and behaviorally monitored via sensory capture devices (e.g., one or more sensors 104). In this regard, with reference again to FIGS. 28 and 29, in one or more additional embodiments, the avatar platform 2802 can further generate avatar versions of animals (e.g., animal 2808) based on image data captured thereof that visually resemble the animals. The avatar animals can comprise two and/or three-dimensional digital models of the animals. The avatar platform 2802 can further configure the visual animal avatars to portray the physical movements, behaviors and physical capabilities of the corresponding animal based on monitored motion data of the animals (e.g., captured/determined via motion sensors and/or based on analysis of video data captured thereof), and physiological performance metrics of the animals (e.g., captured determined via one or more biometric sensors attached to and/or embedded on or within the animal. Using biofeedback captured from sensors 104 and/or analysis of video data of an animal over time, the system can employ machine learning techniques to learn the traits, personality and needs of the animal, facial expressions of the animal, mood of the animal in different contexts (e.g., time of day, environments, etc.) and in response to different stimuli (e.g., people, other animals, food, noise, etc.) energy levels, exhaustion levels and so on. The avatar platform 2802 can also record and learn sounds of the animal (e.g., barking, snoring, whimpering, and any other sounds the animal makes relative to different contexts and stimuli). The avatar platform 2802 can further generate an avatar model of the animal that behaves, performs and sounds in a same or similar manner as the animal in real-life in similar contexts (e.g., at similar times of day, in response to similar stimuli, and so on). In this sense, the avatar platform 2802 can generate a digital version of your pet that not only looks like your pet but acts, behaves and sounds as your pet does in real-life.

In accordance with these embodiments, the animal avatars can also be regarded as intellectual property of their corresponding owners and/or the avatar system. In this regard, the visual appearance of the animal avatar (e.g., computer code defining the visual appearance and/or and image data defining two-dimensional and three-dimensional image features thereof), the sounds of the animal avatar, as well as code controlling the behavior of the avatar e.g., algorithms, models, machine learning models and corresponding parameters as adapted/tailored to the animal) can be regarded by the avatar system as intellectual property of the corresponding animal owner and/or the avatar system. The avatar platform 2802 can further associate the animal avatar and its corresponding data that defines and controls the animal avatar with the actual identity of the animal to which it corresponds (e.g., the animal's name, identification tag number, etc.) and provide the owner with authorization to control its usage thereof by other entities (e.g., other systems, other users, and so on) and/or in association with usage by the owner in other XR games and applications.

For example, in some embodiments, the avatar platform 2802 can provide a gaming application wherein users can participate in activities (and other activities) with other users' animal avatars in an AR and/or VR environment. For example, the gaming activity could involve meeting another user's animal avatar and petting or playing with the animal avatar. In another example, the gaming application can involve visiting an AR or VR zoo and meeting and interacting with animal avatars. In another example, the gaming application could involve a first user having their animal avatar meet and play with another animal avatar in an AR or VR environment. Still in another example, the gaming application could involve a group of users having their respective animal avatars compete against one another in a particular animal fitness activity (e.g., a horse race, a dog race, an animal agility course, a hunting game, etc.) wherein the respective animal avatar abilities, behaviors and traits of the corresponding real animals become the competitive elements of the game.

Still in other embodiments, a user can employ their animal avatar in other XR games and environments provided by third-party systems. For example, a user can play other XR games (e.g., VR and/or AR games) provided by other systems using their animal avatar and/or bring their animal avatar along with them as virtual pet or sidekick.

The avatar platform 2802 can also mint and/or authorize the user associated therewith to mint their identity avatar as an NFT on the blockchain 1704. Once minted on the blockchain, the avatar platform 2802 and/or the user can further control permissions regarding usage of their identity avatar NFT by the user as well as other users and systems (e.g., via avatar usage control component 2914). For example, the avatar platform 2802 and/or the user can restrict usage of the avatar identity to only the actual person corresponding to the avatar identity in other XR environments. In some implementations, the avatar system and/or the user can sell their identity avatar NFT, license usage of their avatar NFT by other users (e.g., in association the gamification aspect discussed above), gift usage of their identity avatar NFT by other users and system, regulate usage of the identity avatar NFT by other users and systems and so on, using permissions and agreements defined on the blockchain 1704. In some embodiments, the user and/or the avatar system can perform transactions for the usage of identity avatar NFTs in exchange for fees (e.g., cryptocurrency or another digital asset) which can be deposited into digital wallets of the avatar system and/or the users. Transactions involving the usage of the identity avatar NFTs (e.g., selling, licensing, gifting, granting usage thereof for specific usage scenarios) can be recorded on the blockchain 1704. In some implementations, these transactions can be defined and controlled automatically using smart contracts recorded on the blockchain 1704.

Additionally, or alternatively, the avatar platform 2802 can perform continuous monitoring of an animal in real-time based on sensory feedback captured for the animal via one or more sensors 104 worn by and/or attached to the animal and/or image data captured of the animal. For example, the sensory feedback can provide information regarding heart rate, exhaustion levels, fatigue levels, activity levels, stress levels, mood, appetite, pain, and various other biofeedback indicators. Based on learned patterns and behaviors of the animal observed over time, (e.g., using machine learning), the system can learn behaviors and tendencies of the animal and alert the owner accordingly. For example, the system can learn and notify the owner (e.g., at a client device associated with the owner) when an animal is becoming fatigued, thirsty, hungry, full, depressed, happy, and/or needs to defecate and urinate. In some embodiments, in which the animal has a corresponding animal avatar generated therefore, the system can cause the animal avatar as presented to the owner (e.g., on a device, as an AR object, as a VR object, as a hologram, etc.) to mimic the current behavior and needs of the animal in association with notifying the owner regarding the detected needs and/or mood of the animal. The system can further provide notify the owner regarding the needs and mood of the animal with verbal cues and/or speech corresponding thereto in a manner as if the animal was actually talking to the owner. For example, the system can cause the animal avatar to speak and state its current needs and mood (e.g., “I'm hungry, “I'm thirsty, “I want to go for a walk,” “I want to play ball,” “I'm getting tired,” “I miss my owner,” etc.).

### XI—Example Operating Environments

The systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which may be explicitly illustrated in this disclosure.

With reference to FIG. 35, a suitable environment 3500 for implementing various aspects of the claimed subject matter includes a computer 3502. The computer 3502 includes a processing unit 3504, a system memory 3506, a codec 3505, and a system bus 3508. The system bus 3508 couples system components including, but not limited to, the system memory 3506 to the processing unit 3504. The processing unit 3504 can be any of various available processors. Dual microprocessors and other multiprocessor architectures also can be employed as the processing unit 3504.

The system bus 3508 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 104), and Small Computer Systems Interface (SCSI).

The system memory 3506 includes volatile memory 10 and non-volatile memory 3512. The basic input/output system (BIOS), containing the basic routines to transfer information between elements within the computer 3502, such as during start-up, is stored in non-volatile memory 3512. In addition, according to present innovations, codec 3505 may include at least one of an encoder or decoder, wherein the at least one of an encoder or decoder may consist of hardware, a combination of hardware and software, or software. Although, codec 3505 is depicted as a separate component, codec 3505 may be contained within non-volatile memory 3512. By way of illustration, and not limitation, non-volatile memory 3512 can include read only memory (ROM), programmable ROM (PROM), electrically programmable ROM (EPROM), electrically erasable programmable ROM (EEPROM), or flash memory. Volatile memory 10 includes random access memory (RAM), which acts as external cache memory. According to present aspects, the volatile memory may store the write operation retry logic (not shown in FIG. 35) and the like. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM.

Computer 3502 may also include removable/non-removable, volatile/non-volatile computer storage medium. FIG. 35 illustrates, for example, disk storage 3511. Disk storage 3511 includes, but is not limited to, devices like a magnetic disk drive, solid state disk (SSD) floppy disk drive, tape drive, Jaz drive, Zip drive, LS-70 drive, flash memory card, or memory stick. In addition, disk storage 3511 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 3511 to the system bus 3508, a removable or non-removable interface is typically used, such as interface 3516.

It is to be appreciated that FIG. 35 describes software that acts as an intermediary between users and the basic computer resources described in the suitable operating environment 3500. Such software includes an operating system 3518. Operating system 3518, which can be stored on disk storage 3511, acts to control and allocate resources of the computer system 3502. Applications 3520 take advantage of the management of resources by operating system 3518 through program modules 3524, and program data 3526, such as the boot/shutdown transaction table and the like, stored either in system memory 3506 or on disk storage 3511. It is to be appreciated that the claimed subject matter can be implemented with various operating systems or combinations of operating systems.

A user enters commands or information into the computer 3502 through input device(s) 3528. Input devices 3528 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 3504 through the system bus 3508 via interface port(s) 3530. Interface port(s) 3530 include, for example, a serial port, a parallel port, a game port, and a universal serial bus (USB). Output device(s) 3536 use some of the same type of ports as input device(s). Thus, for example, a USB port may be used to provide input to computer 3502, and to output information from computer 3502 to an output device 3536. Output adapter 3534 is provided to illustrate that there are some output devices 3536 like monitors, speakers, and printers, among other output devices 3536, which require special adapters. The output adapters 3534 include, by way of illustration and not limitation, video and sound cards that provide a means of connection between the output device 3536 and the system bus 3508. It should be noted that other devices and/or systems of devices provide both input and output capabilities such as remote computer(s) 3538.

Computer 3502 can operate in a networked environment using logical connections to one or more remote computers, such as remote computer(s) 3538. The remote computer(s) 3538 can be a personal computer, a server, a router, a network PC, a workstation, a microprocessor based appliance, a peer device, a smart phone, a tablet, or other network node, and typically includes many of the elements described relative to computer 3502. For purposes of brevity, only a memory storage device 3540 is illustrated with remote computer(s) 3538. Remote computer(s) 3538 is logically connected to computer 3502 through a network interface 3542 and then connected via communication connection(s) 3544. Network interface 3542 encompasses wire and/or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 3544 refers to the hardware/software employed to connect the network interface 3542 to the bus 3508. While communication connection 3544 is shown for illustrative clarity inside computer 3502, it can also be external to computer 3502. The hardware/software necessary for connection to the network interface 3542 includes, for exemplary purposes only, internal and external technologies such as, modems including regular telephone grade modems, cable modems and DSL modems, ISDN adapters, and wired and wireless Ethernet cards, hubs, and routers.

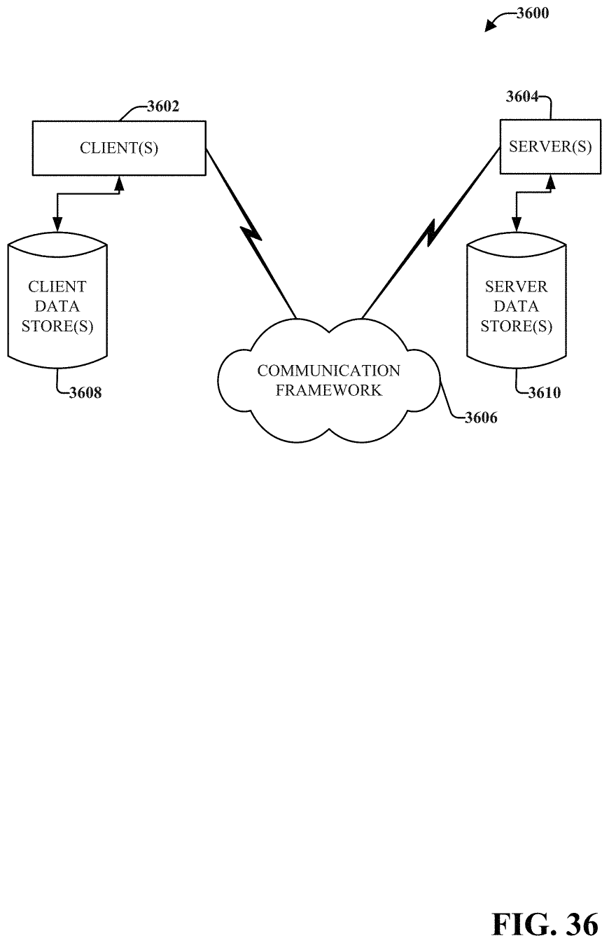

Referring now to FIG. 36, there is illustrated a schematic block diagram of a computing environment 3600 in accordance with this disclosure. The system 3600 includes one or more client(s) 3602 (e.g., laptops, smart phones, PDAs, media players, computers, portable electronic devices, tablets, and the like). The client(s) 3602 can be hardware and/or software (e.g., threads, processes, computing devices). The system 3600 also includes one or more server(s) 3604. The server(s) 3604 can also be hardware or hardware in combination with software (e.g., threads, processes, computing devices). The servers 3604 can house threads to perform transformations by employing aspects of this disclosure, for example. One possible communication between a client 3602 and a server 3604 can be in the form of a data packet transmitted between two or more computer processes wherein the data packet may include video data. The data packet can include a metadata, e.g., associated contextual information, for example. The system 3600 includes a communication framework 3606 (e.g., a global communication network such as the Internet, or mobile network(s)) that can be employed to facilitate communications between the client(s) 3602 and the server(s) 3604.

Communications can be facilitated via a wired (including optical fiber) and/or wireless technology. The client(s) 3602 include or are operatively connected to one or more client data store(s) 3608 that can be employed to store information local to the client(s) 3602 (e.g., associated contextual information). Similarly, the server(s) 3604 are operatively include or are operatively connected to one or more server data store(s) 3610 that can be employed to store information local to the servers 3604.

In one embodiment, a client 3602 can transfer an encoded file, in accordance with the disclosed subject matter, to server 3604. Server 3604 can store the file, decode the file, or transmit the file to another client 3602. It is to be appreciated, that a client 3602 can also transfer uncompressed file to a server 3604 and server 3604 can compress the file in accordance with the disclosed subject matter. Likewise, server 3604 can encode video information and transmit the information via communication framework 3606 to one or more clients 3602.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described in this description can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the disclosure illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer readable storage medium; software transmitted on a computer readable transmission medium; or a combination thereof.

Moreover, the words “example” or “exemplary” are used in this disclosure to mean serving as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used in this description differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described in this disclosure. Furthermore, not all illustrated acts may be required to implement the methodologies in accordance with certain aspects of this disclosure. In addition, those skilled in the art will understand and appreciate that the methodologies could alternatively be represented as a series of interrelated states via a state diagram or events. Additionally, it should be appreciated that the methodologies disclosed in this disclosure are capable of being stored on an article of manufacture to facilitate transporting and transferring such methodologies to computing devices. The term article of manufacture, as used in this disclosure, is intended to encompass a computer program accessible from any computer-readable device or storage media.

What is claimed is:

1. A system comprising:
a non-transitory memory comprising instructions; and
one or more hardware processors coupled to the non-transitory memory and configured to read the instructions to cause the system to perform operations comprising:
receiving health and fitness information for entities;
employing a blockchain network to store the health and fitness information for the entities; and
sending a non-fungible token to an entity of the entities in response to a subset of the health and fitness information associated with the entity satisfying a criterion.

2. The system of claim 1, wherein the sending comprises sending the non-fungible token to an address on the blockchain network associated with the entity.

3. The system of claim 1, wherein the sending is responsive to self-execution of a smart contract as a function of the subset of the health and fitness information becoming stored on the blockchain network, and wherein the smart contract is encoded on the blockchain network.

4. The system of claim 1, wherein the non-fungible token corresponds to an item selected from the group consisting of: a digital collectible, a digital badge, a digital coupon and a digital avatar item or accessory.

5. The system of claim 1, wherein the non-fungible token corresponds to a digital access pass to a real-world event or environment.

6. The system of claim 1, wherein the non-fungible token corresponds to a digital access pass to a virtual reality environment, an augmented reality environment, or a mixed reality environment.

7. The system of claim 1, wherein the health and fitness information relates to performance of one or more fitness activities.

8. The system of claim 1, wherein the health and fitness information comprises physical fitness performance metrics.

9. The system of claim 1, wherein the receiving comprises receiving the health and fitness information from one or more authorized data sources, and wherein the employing comprises storing the health and fitness information on the blockchain network in response to the receiving.

10. The system of claim 9, wherein the operations further comprise:
linking respective portions of the health and fitness information corresponding to respective entities of the entities with respective addresses of the entities of the blockchain network.

11. The system of claim 1, wherein the operations further comprise:
generating a digital avatar for an entity of the entities based on image data captured of the entity; and
minting a second non-fungible token for the digital avatar and the entity on the blockchain network.

12. The system of claim 11, wherein the operations further comprise:
controlling usage of the digital avatar by a user identity in association with usage of the digital avatar in one or more virtual reality, augmented reality, or extended reality environments based on verification that the user identity corresponds to the entity and verification that the second non-fungible token is owned by the entity via the blockchain network.

13. A method, comprising:
receiving, by a system comprising a processor, health and fitness information for entities;
employing, by the system, a blockchain network to store the health and fitness information for the entities; and
sending, by the system, a non-fungible token to an entity of the entities in response to a subset of the health and fitness information associated with the entity satisfying a criterion.

14. The method of claim 13, wherein the sending comprises sending the non-fungible token to an address on the blockchain network associated with the entity.

15. The method of claim 13, wherein the sending is responsive to self-execution of a smart contract as a function of the subset of the health and fitness information becoming stored on the blockchain network, and wherein the smart contract is encoded on the blockchain network.

16. The method of claim 13, wherein the non-fungible token corresponds to an item selected from the group consisting of: a digital collectible, a digital badge, a digital coupon and a digital avatar item or accessory.

17. The method of claim 13, wherein the receiving comprises receiving the health and fitness information from one or more authorized data sources, and wherein the employing comprises storing the health and fitness information on the blockchain network in response to the receiving, and wherein the method further comprises:
linking, by the system, respective portions of the health and fitness information corresponding to respective entities of the entities with respective addresses of the entities of the blockchain network.

18. The method of claim 13, further comprising:
generating, by the system, a digital avatar for an entity of the entities based on image data captured of the entity; and
minting, by the system, a second non-fungible token for the digital avatar and the entity on the blockchain network.

19. The method of claim 18, further comprising:
controlling, by the system, usage of the digital avatar by a user identity in association with rendering the digital avatar in one or more virtual reality, augmented reality, or extended reality environments based on verification that the user identity corresponds to the entity and verification of the second non-fungible token via the blockchain network.

20. A non-transitory machine-readable storage medium, comprising executable instructions that, when executed by a processor, facilitate performance of operations, comprising:
receiving health and fitness information for entities;
employing a blockchain network to store the health and fitness information for the entities; and
sending a non-fungible token to an entity of the entities in response to a subset of the health and fitness information associated with the entity satisfying a criterion.
